SEQUENCE LISTING (62 sequences)

SEQ ID NO: 1 - DNA, 5116 bp, Zea mays
  source 1..5116: mol_type = unassigned DNA
ctcggctcatcagtattattattattattattattcggctcctgctcatcagctgcagcagtcgtgctccggaccggagaagtcgaaatggaggagaggctgttcgccacggagaagcacggtggccgggcgctctacaggctccacgccgtcacggtgttcctggggatatgcctgctgctctgctacagggcgacgcacgtcccggctgccggctccggcggcagggcggcgtggctggggatgctcgcggcggagctctggttcggcttctactgggtcatcacgcagtccgtgcgctggtgccccatccgccgccgcaccttccacgacaggctcgccgccaggtctgtgacttgaccttcttcgtgtgcatgcacatatacaaactattttgcttgttgcacgtccagtcatttttcaaaggaaggctaatatgagttaataatgaggctaactggtggtcatagtccactaactctctctatatattagcttaatacatgtaagtatcacacggtcacacacccctataagtacatagaaactagcagttccaacaacatcttatgttacaaatgtttatataaaaaaatataggacacactataaagtgacctaaatacaccacacctcatattaatgtcatattcatattttctatatcattatcaacatttttcttttctaataatgcaatttactttcttaaatacatgatatagagcttaacgattggagctcaatttattttcagtgccctgaacactttaaatgatgtagtattttaatttttgggaaattatttttgagacacctatttggagatgatctgcacatacactctcgtttcttgagggtttaaacccatgactcttatactccctccattccatattaaaattcgttttagttaattaatcggtttatacaatatttagtatatatatgtttaaatctatcttcaaatatttgaatatggacataaaaattaagagctaaactaactacgaatggatgagtatatattactaaaaaatgaattccaaacgatggtcgttcacaaggtgagccgtttctatacatgagctgggttcggaatgtggactgtggaagctagaagttgcgacagccgataggctgtgggggcaaacaaatctaagtatgaaaccgtgcgtgctacagactgcagaccacaggcacgcaggaggagcagataagatagactcgtgcagtggcgatgcatcagctgattggggggtccattcaggtccttctgcagcatatataagcgccgcgtgcagccaaattaaaacgatggatatggcacgggttcttctgactgcaccatgcctgtctgttttgttctactgactactgcctaagacgtcgaggccaaggccactgtgtagctcgcgagattgtttactgcagaaggcagttgggccacgtgggctctatcgcacgccttggtgtgggggttgtcgttcagcgctacggctgtcgacgacggtgcgtttcaatgatgccgtcggcgacggtggtgctggtggcctgtgtggctgtggtgtcacgggtttctaactctagcgccagttgcaccaatgcttttagggctcgtttgggacataggagtttcccccccactatttcatgtgttttgctacgaaaataaactgatttcagtgaaattcgtgtatgattcacctaaaatttctgcattccaaatgatacattattagatttggctccatgcggcccacaccataggatgtgacttgaagatggtgtccttgggcgcatgttctcttgaaccatttattttcgaggaattaaaatttattcaataaagtgatctatttgggttattggaatttaacattttatcactttttcagatataagactattttaaattcatgcggtggaggacgtgaaatgatattatgtattactagaatatgtttctactctgcaacttacacgacgcacttcgacttattttctctgccgtaaaatgtattttaaataatagtatacaaatatattttaaataaaactatattagcctaaattagtatcgttagaatggaattcaattccagtaaggtgcgtcccccttcgatctccgttggtccatatgaaactgaagctaattaataaagtcaagacaccatccaacaggtggtgtgtgtgcgtcgtaatgcctgcgaggaggttggctgctaaacccagccagagcctcctatgatactgtccaggcacaaaatatcctttggtgatgcacaagtcgggccggggtcacgaacatggcacagagcctggactcagggtttcggcataaactataccatctttgctgctccattgtcggcctcaagctcggacattggctgatactgctttgattgccgtcgtacgttaacattattggtatcatgtcggagcaggttcggagagcggctcccctgcgtggacatcttcgtgtgcacagcggacccgcggtcggagccgccgagccttgtcgtggccacggtcctgtcggtgatggcgtacaactacccgcccgcgaagctcaacgtctacctctccgacgacggcggctccatcctcaccttctacgctctgtgggaggcctccgccttcgccaagcactggctcccgttctgcaggaggtacggcgtcgagccacggtcgccggccgcttacttcgcccagtctgatgagaagcctcgtcatgatccgccgcacgccttgcaggagtggacgtccgtcaaagtacgtgcacgcgtgtgtttttactgtgtatagacggacggatgcatgtcttgctagctagcttgtaggcagcgtgtggcaacgaactgatatttcttctggtccgcgcagaacctatacgatgaaatgacggagcggattgactccgctgctcggacgggcaatgttcctgaagaaactagagcgaaacacaaagggttttctgagtgggatacgggtattacctcaaaagaccaccacccgatcgttcaggtatatatatcattattcccgtccctctatatttctccagacttttttgtattataaaaatatagatgatgttttcttttgctagattctgatagatgggaaagacaaggctgtagctgacaacgaaggcaatgtgctgccgacgctggtgtacgtggcacgagagaagaggcctcagtaccaccacaacttcaaagccggggcgatgaacgctctggtatgattcattcattcgcaccagctggtagcttagcatgcagggacattgtttgcttaacatatttataatatctgtgcgagatcgccgccaatttgaactgcagatccgagtatcgtccgtgataagcaacagccctatcatcctgaacgtggactgcgacatgtattccaacaacagcgacacgatcagagacgcgctgtgcttcttcctcgacgaagaaacgggccacaggatcgcgttcgtgcagtaccctcagaactacaacaacctcaccaagaacaacatatacggcaactccctcaatgtcatcaaccaggttagtactgtcaagtactgaaattatatatgcatgcctcttgacagcgacactgacaatgttgggcggcgctgaatcatcacaaggtggagctgagcggcctggacgcttggggcggcccgctgtacatcggcacgggatgcttccataggagggagaccctgtgcggcaggaggttcaccgaggactacaaggaagactgggacagaggaaccaaggagcagcagcagcaccgccaccgcgtcgacggcgagaccgaagcgaaggccaagtcgctagcgacctgcgcctacgagcacgacgacgacacgcggtggggagacgaggtggggctcaagtacggctgctcggtggaggacgtcatcacggggctggcgatacactgcagagggtgggagtcggtgtacagcaaccccgcgagagcggcgttcgtcggcgtcgcgccgaccacgctcgcccagaccatactgcagcacaagcggtggagcgagggcaacttcggcatcttcgtttccaggtactgccccttcgtctttggacgacggggcaaaaccaggttgccgcaccagatgggctactccatctacgggctatgggcgcccaactcgctgcctacgctgtactacgctgtcgtcccttcgctgtgcctgctcaagggcacccctctgttccctgaggtatgcatgtcgtacgtgtgattcaatggcattgaagcatatatatgtgctctctcttgagtcttgactgtgtgtgtgtgtttgtttcatcagctcacgagtccgtggatcgcgcctttcgtctacgtcgcggtcgccaagaacgtctacagcgcgtgggaggcgctgtggtgcggagacacgctgagagggtggtggaacgggcagaggatgtggctggtccggagaacgacctcgtacctctacggcttcgtcgacaccgtcagggactcgctggggctgtccaagatgggcttcgtggtgtcgtccaaggtgagcgacgaggacgaggccaagaggtacgagcaggagatgatggagttcgggacggcgtcgccggagtacgtgatcgtcgcggccgtcgcgctgctcaacctcgtgtgcctggcagggatggcggcggcactggatgtgttcttcgtccaggtcgctctctgcggggtgctggtgctcctcaacgtcccggtctatgaagccatgttcgtcaggaaggacagggggaggatgccgttcccgatcacgctagcctccgttggctttgtgacgctggccctcattgtgccattcttttgactttgaggtgctaataatacgtgtacgggcacacgcacgttcgcatgtatgacgattatgggcaacaggcgtgtaataccactaatacctattaaacactccagtctccaagtgatccattgctacacgtgtgttcctcctgttctctatatgcatgagctgctgatggtgatacgatactgtcagatactgcaataagacgccaaacaagataagcatcggcaatcgagtggatcctcacaaccacagtggacgctatgc

SEQ ID NO: 2 - DNA, 2184 bp, synthetic construct
  misc_feature 1..2184: note = cDNA of ZmCSLE derived from B73
  source 1..2184: mol_type = other DNA
atggaggagaggctgttcgccacggagaagcacggtggccgggcgctctacaggctccacgccgtcacggtgttcctggggatatgcctgctgctctgctacagggcgacgcacgtcccggctgccggctccggcggcagggcggcgtggctggggatgctcgcggcggagctctggttcggcttctactgggtcatcacgcagtccgtgcgctggtgccccatccgccgccgcaccttccacgacaggctcgccgccaggttcggagagcggctcccctgcgtggacatcttcgtgtgcacagcggacccgcggtcggagccgccgagccttgtcgtggccacggtcctgtcggtgatggcgtacaactacccgcccgcgaagctcaacgtctacctctccgacgacggcggctccatcctcaccttctacgctctgtgggaggcctccgccttcgccaagcactggctcccgttctgcaggaggtacggcgtcgagccacggtcgccggccgcttacttcgcccagtctgatgagaagcctcgtcatgatccgccgcacgccttgcaggagtggacgtccgtcaaaaacctatacgatgaaatgacggagcggattgactccgctgctcggacgggcaatgttcctgaagaaactagagcgaaacacaaagggttttctgagtgggatacgggtattacctcaaaagaccaccacccgatcgttcagattctgatagatgggaaagacaaggctgtagctgacaacgaaggcaatgtgctgccgacgctggtgtacgtggcacgagagaagaggcctcagtaccaccacaacttcaaagccggggcgatgaacgctctgatccgagtatcgtccgtgataagcaacagccctatcatcctgaacgtggactgcgacatgtattccaacaacagcgacacgatcagagacgcgctgtgcttcttcctcgacgaagaaacgggccacaggatcgcgttcgtgcagtaccctcagaactacaacaacctcaccaagaacaacatatacggcaactccctcaatgtcatcaaccaggtggagctgagcggcctggacgcttggggcggcccgctgtacatcggcacgggatgcttccataggagggagaccctgtgcggcaggaggttcaccgaggactacaaggaagactgggacagaggaaccaaggagcagcagcagcaccgccaccgcgtcgacggcgagaccgaagcgaaggccaagtcgctagcgacctgcgcctacgagcacgacgacgacacgcggtggggagacgaggtggggctcaagtacggctgctcggtggaggacgtcatcacggggctggcgatacactgcagagggtgggagtcggtgtacagcaaccccgcgagagcggcgttcgtcggcgtcgcgccgaccacgctcgcccagaccatactgcagcacaagcggtggagcgagggcaacttcggcatcttcgtttccaggtactgccccttcgtctttggacgacggggcaaaaccaggttgccgcaccagatgggctactccatctacgggctatgggcgcccaactcgctgcctacgctgtactacgctgtcgtcccttcgctgtgcctgctcaagggcacccctctgttccctgagctcacgagtccgtggatcgcgcctttcgtctacgtcgcggtcgccaagaacgtctacagcgcgtgggaggcgctgtggtgcggagacacgctgagagggtggtggaacgggcagaggatgtggctggtccggagaacgacctcgtacctctacggcttcgtcgacaccgtcagggactcgctggggctgtccaagatgggcttcgtggtgtcgtccaaggtgagcgacgaggacgaggccaagaggtacgagcaggagatgatggagttcgggacggcgtcgccggagtacgtgatcgtcgcggccgtcgcgctgctcaacctcgtgtgcctggcagggatggcggcggcactggatgtgttcttcgtccaggtcgctctctgcggggtgctggtgctcctcaacgtcccggtctatgaagccatgttcgtcaggaaggacagggggaggatgccgttcccgatcacgctagcctccgttggctttgtgacgctggccctcattgtgccattcttttga

SEQ ID NO: 3 - protein, 727 aa, Zea mays
MEERLFATEKHGGRALYRLHAVTVFLGICLLLCYRATHVPAAGSGGRAAWLGMLAAELWFGFYWVITQSVRWCPIRRRTFHDRLAARFGERLPCVDIFVCTADPRSEPPSLVVATVLSVMAYNYPPAKLNVYLSDDGGSILTFYALWEASAFAKHWLPFCRRYGVEPRSPAAYFAQSDEKPRHDPPHALQEWTSVKNLYDEMTERIDSAARTGNVPEETRAKHKGFSEWDTGITSKDHHPIVQILIDGKDKAVADNEGNVLPTLVYVAREKRPQYHHNFKAGAMNALIRVSSVISNSPIILNVDCDMYSNNSDTIRDALCFFLDEETGHRIAFVQYPQNYNNLTKNNIYGNSLNVINQVELSGLDAWGGPLYIGTGCFHRRETLCGRRFTEDYKEDWDRGTKEQQQHRHRVDGETEAKAKSLATCAYEHDDDTRWGDEVGLKYGCSVEDVITGLAIHCRGWESVYSNPARAAFVGVAPTTLAQTILQHKRWSEGNFGIFVSRYCPFVFGRRGKTRLPHQMGYSIYGLWAPNSLPTLYYAVVPSLCLLKGTPLFPELTSPWIAPFVYVAVAKNVYSAWEALWCGDTLRGWWNGQRMWLVRRTTSYLYGFVDTVRDSLGLSKMGFVVSSKVSDEDEAKRYEQEMMEFGTASPEYVIVAAVALLNLVCLAGMAAALDVFFVQVALCGVLVLLNVPVYEAMFVRKDRGRMPFPITLASVGFVTLALIVPFF

SEQ ID NO: 4 - DNA, 5274 bp, Zea mays
  source 1..5274: mol_type = unassigned DNA
ctcggctcatcagtattattattattattattattcggctcctgctcatcagctgcagcagtcgtgctccggaccggagaagtcgaaatggaggagaggctgttcgccacggagaagcacggtggccgggcgctctacaggctccacgccgtcacggtgttcctggggatatgcctggtgctctgctacagggcgacgcacgtcccggctgccggctccggcggcagggcggcgtggctggggatgctcgcggcggagctctggttcggcttctactgggtcatcacgcagtccgtgcgctggtgccccatccgccgccgcaccttccacgacaggctcgccgccaggtttgccacttgacgaccttcttcgtgtgcatgcacatatacaaactattatccttgttgcacgtccggtcatttttcaaaggaaggctaatatgagttactaatgaggctaactggtggtcatatatagtccacactaactctctctatatattagcttaatacatgtaagtatcacacggtcacacacccctataagtacaagtgcatatgagcatcttatcttaaatcagtgctctatcttagaataatatagagcacaatcataaaaaacagttcatagaataatatagagcacaatcataaaaaacagttcaacaacatcttatgttacaaatatttatataaaaaaaatataggacacgctataaagtgacccaaatataacacgtctcatattagtgtcatattcttattttctatatcattatcaacattttcttttctaataatgcaatttacttcctcaaatacatgatatagagcttaacgattgtagctcaatttattttcagtgtcctgaacactttggatgatgtagtatttaatttttgggaaattattttgagacacctattcggagatgctctgcacatacactctcgtttcttgagggtttaaacccgtgacaatgcgtgaccccaaaccgtttctcttatactccctccgttttcaaattaaaattcgttttatttaattaatgggtttatataatatttggtatatttgtctagatctatcttcgaatacttaatatagatataaaaatcaagacctaaaccaactactattttagaatggatgaggagtatatattactaaaaaaaaattgaatttgaaacgatggttcacaaggtgagccgtttctacagctgggttcggaatgtggactgtggagtcctactctttcattccagtggcagctagaactagaagtttgcgacagccgacaggctgtgggggcaaacaaatctaagtatgaaatgaaaccgtgcgtgctacagactgcagaccacaggcacgcaggaggagcagataacagatagactcgtggggatgcatcagctgattgggggtccattcaggtccttctgcagcatatataagcgccgcgtgcagccaaattaaaacgatggatatggcatgggttcttctgactgcaccatgcctgtctgttttgttctactgactaactacagtttcccctctcgcgctgcctaagacgtcgaggccaaggccactgtgtagctagctcgcgagatttgttactgcagatggcagttgggccacgtgggctctatcgcacgccttggtgtgggggttgtcgttcagcgctacggctgtcgacgacggatgcgtttcaatgatgccgtcggcgacggtggtgctggtggcgtgtgtggctgtggtgtcacgggcttctaattctagcgccagttgcaccaatgcttttagggctcgtttggaacgtaggattgcccccccactatttcatgtttttttcctacgaaaaagaactgatttcagtgacattcctgcatctaaaatttccgcattccaaatgatacattagatttgtctccatgcggcccacaccataggatgtgacttgaagatggtgtccttgggcgcatgttctctggatccatttattttcgaggaattaaaatttattcaataaagtgatctatttgagttattggaatttaacattttatcactttttcagatataagactattttaaatttatgtggtggaggatgtgaaatgatattatatatcactagaatatgtttctactctgtaacttacacggcacacttcaactcattttctctaccgcaaaaatgtagcacataaaaacatttaacatcttgatgataataatatataaatatattttgaataaaattgtattagtctaaattagtatcgttagaatggaattcaattccaaggatctacctttgtagtaatgtgcgtcccccttcgatctccgttggtccgcatgaaactgaagctaattaggaaagtccaagacaccgtccatggtggtgtgtgcgcgtcctagtaatgcctgcgaggaggttggctgctaaacccagccagagcctcctatcatactgtccagtccaggcacaaaatatcctttggtgatgcacaagtcgggggtcacgaacatggctctctgcagggttcaggctcggacacattttggctgatactgcttcgttgattgtcggagcaggttcggagaacggctcccctgcgtggacatcttcgtgtgcacagcggacccgcggtcggagccgccgagccttgtcgtggccacggtcctgtcggtgatggcgtacaactacccgcccgcgaagctcaacgtgtacctctccgacgacggcggctccatcctcaccttctacgctatgtgggaggcctccgccttcgccaagcactggctcccgttctgcaggaggtacggcgtcgagccacggtcgccggccgcttacttcgcccagtcagatgagaagcctcgtcatgatccgccgcacgccttgcaggagtggacgtccgtcaaagtacgtgcacgcgtttgtttttactgtgtatagacggacggatgcatgtctagctagctagcttgtaggcagcgtgcacgtggcaacgaactgatatttcttctggtccgcgcagaacctatacgatgaaatgacggagcggattgactccgctgctaggacgggcaatgttcctgaagaaactagagcgaaacacaaagggttttctgagtgggatacgggtattacctcaaaagaccaccacccgatcgttcaggtatatatatcattattcccgtccctctatatttctccagacttttttgtattataaaaatatagatgatgttttcttttgctagattctgatagatgggaaagacaaggctgtagctgacaacgaaggcaatgtgctgccgacgctggtgtacgtggcacgagagaagaggcctcagtaccaccacaacttcaaagccggggcgatgaacgctctggtatgattcattcattcgcaccagctggtagcttagcatgcagggacattgtttgcttaacatatttataatatctgtgcgagatcgccgccaatttgaactgcagatccgagtatcgtccgtgataagcaacagccctatcatcctgaacgtggactgcgacatgtactccaacaacagcgacacgatcagagacgcgctgtgcttcttcctcgacgaagaaacgggccacaggatcgcgttcgtgcagtaccctcagaactacaacaacctcaccaagaacaacatatacggcaactccctcaatgtcatcaaccaggttagtgtcgagtactgaaattatatatgcatgcctcttgacagcgacactgacactgacactgttgggcggcgctgaatcatcacaaggtggagctgagcggcctggacgcttggggcggcccgctgtacatcggcacgggatgcttccataggagggagaccctgtgcggcaggaggttcaccgaggactacaaggaagactgggacagaggaaccaaggagcagcagcagcagcaccgcgtcgacggcgagaccgaagcgaaggccaagtcgctagcgacctgcgcctacgagcacgacgacgaggacacgcggtggggagacgaggtggggctcaagtacggctgctcggtggaggacgtcatcacggggctggcgatacactgcagagggtgggagtcggtgtacagcaaccccgcgagagcggcgttcgtcggcgtcgcgccgaccacgctcgcgcagaccatactgcagcacaagcggtggagcgagggcaacttcggcatcttcgtttccaggtactgccccttcgtctttggacgacggggcaaaaccaggttgccgcaccagatgggctactccatctacgggctatgggcgcccaactcgctgcctacgctgtactacgctgtcgtcccttcgctgtgcctgctcaagggcacccctctgttccctgaggtatatatgcatgtcgtacgtgtgattcaatggcattgaagcatatatatgtgctctctcttgagtcttgactgtgtgtgtgtgtttgtttcatcagctcacgagtccgtggatcgcgcctttcgtctacgtcgcggtcgccaagaacgtctacagcgcgtgggaggcgctgtggtgcggagacacgctgagagggtggtggaacgggcagaggatgtggctggtccggagaacgacctcgtacctctacggcttcgtcgacaccgtcagggactcgctggggctgtccaagatgggcttcgtggtgtcgtccaaggtgagcgacgaggacgaggccaagaggtacgagcaggagatgatggagttcgggacggcgtcgccggagtacgtgatcgtcgcggccgtcgcgctgctcaacctcgtgtgcctggcagggatggcggcggcactggatgtgttcttcgtccaggtcgctctctgcggggtgctggtgctcctcaacgtcccggtctatgaagccatgttcgtcaggaaggacagggggaggatgccgttcccgatcacgctagcctccgttggctttgtgacgctggccctcattgtgccattcttttgactttgaggtgctaataatacgtgtacgggcacacgcacgttcgcatgtatgacgattatgggcaacaggcgtgtaataccactaatacctattaaacactccagtctccaagtgatccattgctacacgtgtgttcctcctgttctctatatgcatgagctgctgatggtgatacgatactgtcagatactgcaataagacgccaaacaagataagcatcggcaatcgagtggatcctcacaaccacagtggacgctatgc

SEQ ID NO: 5 - DNA, 2184 bp, synthetic construct
  misc_feature 1..2184: note = cDNA of ZmCSLE derived from PH207
  source 1..2184: mol_type = other DNA
atggaggagaggctgttcgccacggagaagcacggtggccgggcgctctacaggctccacgccgtcacggtgttcctggggatatgcctggtgctctgctacagggcgacgcacgtcccggctgccggctccggcggcagggcggcgtggctggggatgctcgcggcggagctctggttcggcttctactgggtcatcacgcagtccgtgcgctggtgccccatccgccgccgcaccttccacgacaggctcgccgccaggttcggagaacggctcccctgcgtggacatcttcgtgtgcacagcggacccgcggtcggagccgccgagccttgtcgtggccacggtcctgtcggtgatggcgtacaactacccgcccgcgaagctcaacgtgtacctctccgacgacggcggctccatcctcaccttctacgctatgtgggaggcctccgccttcgccaagcactggctcccgttctgcaggaggtacggcgtcgagccacggtcgccggccgcttacttcgcccagtcagatgagaagcctcgtcatgatccgccgcacgccttgcaggagtggacgtccgtcaaaaacctatacgatgaaatgacggagcggattgactccgctgctaggacgggcaatgttcctgaagaaactagagcgaaacacaaagggttttctgagtgggatacgggtattacctcaaaagaccaccacccgatcgttcagattctgatagatgggaaagacaaggctgtagctgacaacgaaggcaatgtgctgccgacgctggtgtacgtggcacgagagaagaggcctcagtaccaccacaacttcaaagccggggcgatgaacgctctgatccgagtatcgtccgtgataagcaacagccctatcatcctgaacgtggactgcgacatgtactccaacaacagcgacacgatcagagacgcgctgtgcttcttcctcgacgaagaaacgggccacaggatcgcgttcgtgcagtaccctcagaactacaacaacctcaccaagaacaacatatacggcaactccctcaatgtcatcaaccaggtggagctgagcggcctggacgcttggggcggcccgctgtacatcggcacgggatgcttccataggagggagaccctgtgcggcaggaggttcaccgaggactacaaggaagactgggacagaggaaccaaggagcagcagcagcagcaccgcgtcgacggcgagaccgaagcgaaggccaagtcgctagcgacctgcgcctacgagcacgacgacgaggacacgcggtggggagacgaggtggggctcaagtacggctgctcggtggaggacgtcatcacggggctggcgatacactgcagagggtgggagtcggtgtacagcaaccccgcgagagcggcgttcgtcggcgtcgcgccgaccacgctcgcgcagaccatactgcagcacaagcggtggagcgagggcaacttcggcatcttcgtttccaggtactgccccttcgtctttggacgacggggcaaaaccaggttgccgcaccagatgggctactccatctacgggctatgggcgcccaactcgctgcctacgctgtactacgctgtcgtcccttcgctgtgcctgctcaagggcacccctctgttccctgagctcacgagtccgtggatcgcgcctttcgtctacgtcgcggtcgccaagaacgtctacagcgcgtgggaggcgctgtggtgcggagacacgctgagagggtggtggaacgggcagaggatgtggctggtccggagaacgacctcgtacctctacggcttcgtcgacaccgtcagggactcgctggggctgtccaagatgggcttcgtggtgtcgtccaaggtgagcgacgaggacgaggccaagaggtacgagcaggagatgatggagttcgggacggcgtcgccggagtacgtgatcgtcgcggccgtcgcgctgctcaacctcgtgtgcctggcagggatggcggcggcactggatgtgttcttcgtccaggtcgctctctgcggggtgctggtgctcctcaacgtcccggtctatgaagccatgttcgtcaggaaggacagggggaggatgccgttcccgatcacgctagcctccgttggctttgtgacgctggccctcattgtgccattcttttga

SEQ ID NO: 6 - protein, 727 aa, Zea mays
MEERLFATEKHGGRALYRLHAVTVFLGICLVLCYRATHVPAAGSGGRAAWLGMLAAELWFGFYWVITQSVRWCPIRRRTFHDRLAARFGERLPCVDIFVCTADPRSEPPSLVVATVLSVMAYNYPPAKLNVYLSDDGGSILTFYAMWEASAFAKHWLPFCRRYGVEPRSPAAYFAQSDEKPRHDPPHALQEWTSVKNLYDEMTERIDSAARTGNVPEETRAKHKGFSEWDTGITSKDHHPIVQILIDGKDKAVADNEGNVLPTLVYVAREKRPQYHHNFKAGAMNALIRVSSVISNSPIILNVDCDMYSNNSDTIRDALCFFLDEETGHRIAFVQYPQNYNNLTKNNIYGNSLNVINQVELSGLDAWGGPLYIGTGCFHRRETLCGRRFTEDYKEDWDRGTKEQQQQHRVDGETEAKAKSLATCAYEHDDEDTRWGDEVGLKYGCSVEDVITGLAIHCRGWESVYSNPARAAFVGVAPTTLAQTILQHKRWSEGNFGIFVSRYCPFVFGRRGKTRLPHQMGYSIYGLWAPNSLPTLYYAVVPSLCLLKGTPLFPELTSPWIAPFVYVAVAKNVYSAWEALWCGDTLRGWWNGQRMWLVRRTTSYLYGFVDTVRDSLGLSKMGFVVSSKVSDEDEAKRYEQEMMEFGTASPEYVIVAAVALLNLVCLAGMAAALDVFFVQVALCGVLVLLNVPVYEAMFVRKDRGRMPFPITLASVGFVTLALIVPFF

SEQ ID NO: 7 - DNA, 7116 bp, Zea mays
  source 1..7116: mol_type = unassigned DNA
ttcagaacacagtccagcagccgagtgagggtatttccatctcgtgactctgcgcgcacagagaagcgagagggcaggtgcctccggagcccttgccgttcgagaccttgcacgagggatcggcaattaggtttttggggagcgtctacgcgactgcccaaagtcttcctcctgctgacatgacaagtgaagttggacaaggatctagatcctcggagcgcatgggagggtatgatcaattcatctccttactgtttttcattctgcaaattatatatgttcatacctgctgttttatttatagccgtaaatttattcaatctgttttgtcgtattatatcataacatgtctgatgcctgatcatactagtaatatgataaatctgtttgtcttaattttacagtcatgctgtttatgttgctatctgtttattttcagttgttccataataatacatgtttcatgtttatatgcttattatatttatatgattcatatgtttcatgttctcttgatccatattgttatggatatatttgagataatgatttctatgattaaacatattttatatgtcatcatcataatgttaatttatggaattaaaataatacggaaaatgcctatatttctaacaaaatatggtattagaaagtacatattgtattaatatttactataagtttcagcagattgagattgtatactctagataacgatgtttactgtcttcaacatatcatgtacatgatcatataaaatactatactattctacataataaataattataaacagtagagtttgaaatagaaaatcggtgaagacagccttacgctgacactgtcacttacactgaacacctcagtgcacgtgccgtctctgcaacgattagctgcatcggtcgctagccgcccctgtcggcgtacgtatcggcagcgagccaatgacacacgatccatcggctttatatacgccacccgctgctgctctccggtcctcggctcatcagtattattattattattattattcggctcctgctcatcagctgcagcagtcgtgctccggaccggagaagtcgaaatggaggagaggctgttcgccacggagaagcacggtggccgggcgctctacaggctccacgccgtcacggtgttcctggggatatgcctgctgctctgctacagggcgacgcacgtcccggctgccggctccggcggcagggcggcgtggctggggatgctcgcggcggagctctggttcggcttctactgggtcatcacgcagtccgtgcgctggtgccccatccgccgccgcaccttccacgacaggctcgccgccaggtctgtgacttgaccttcttcgtgtgcatgcacatatacaaactattttgcttgttgcacgtccagtcatttttcaaaggaaggctaatatgagttaataatgaggctaactggtggtcatagtccactaactctctctatatattagcttaatacatgtaagtatcacacggtcacacacccctataagtacatagaaactagcagttccaacaacatcttatgttacaaatgtttatataaaaaaatataggacacactataaagtgacctaaatacaccacacctcatattaatgtcatattcatattttctatatcattatcaacatttttcttttctaataatgcaatttactttcttaaatacatgatatagagcttaacgattggagctcaatttattttcagtgccctgaacactttaaatgatgtagtattttaatttttgggaaattatttttgagacacctatttggagatgatctgcacatacactctcgtttcttgagggtttaaacccatgactcttatactccctccattccatattaaaattcgttttagttaattaatcggtttatacaatatttagtatatatatgtttaaatctatcttcaaatatttgaatatggacataaaaattaagagctaaactaactacgaatggatgagtatatattactaaaaaatgaattccaaacgatggtcgttcacaaggtgagccgtttctatacatgagctgggttcggaatgtggactgtggaagctagaagttgcgacagccgataggctgtgggggcaaacaaatctaagtatgaaaccgtgcgtgctacagactgcagaccacaggcacgcaggaggagcagataagatagactcgtgcagtggcgatgcatcagctgattggggggtccattcaggtccttctgcagcatatataagcgccgcgtgcagccaaattaaaacgatggatatggcacgggttcttctgactgcaccatgcctgtctgttttgttctactgactactgcctaagacgtcgaggccaaggccactgtgtagctcgcgagattgtttactgcagaaggcagttgggccacgtgggctctatcgcacgccttggtgtgggggttgtcgttcagcgctacggctgtcgacgacggtgcgtttcaatgatgccgtcggcgacggtggtgctggtggcctgtgtggctgtggtgtcacgggtttctaactctagcgccagttgcaccaatgcttttagggctcgtttgggacataggagtttcccccccactatttcatgtgttttgctacgaaaataaactgatttcagtgaaattcgtgtatgattcacctaaaatttctgcattccaaatgatacattattagatttggctccatgcggcccacaccataggatgtgacttgaagatggtgtccttgggcgcatgttctcttgaaccatttattttcgaggaattaaaatttattcaataaagtgatctatttgggttattggaatttaacattttatcactttttcagatataagactattttaaattcatgcggtggaggacgtgaaatgatattatgtattactagaatatgtttctactctgcaacttacacgacgcacttcgacttattttctctgccgtaaaatgtattttaaataatagtatacaaatatattttaaataaaactatattagcctaaattagtatcgttagaatggaattcaattccagtaaggtgcgtcccccttcgatctccgttggtccatatgaaactgaagctaattaataaagtcaagacaccatccaacaggtggtgtgtgtgcgtcgtaatgcctgcgaggaggttggctgctaaacccagccagagcctcctatgatactgtccaggcacaaaatatcctttggtgatgcacaagtcgggccggggtcacgaacatggcacagagcctggactcagggtttcggcataaactataccatctttgctgctccattgtcggcctcaagctcggacattggctgatactgctttgattgccgtcgtacgttaacattattggtatcatgtcggagcaggttcggagagcggctcccctgcgtggacatcttcgtgtgcacagcggacccgcggtcggagccgccgagccttgtcgtggccacggtcctgtcggtgatggcgtacaactacccgcccgcgaagctcaacgtctacctctccgacgacggcggctccatcctcaccttctacgctctgtgggaggcctccgccttcgccaagcactggctcccgttctgcaggaggtacggcgtcgagccacggtcgccggccgcttacttcgcccagtctgatgagaagcctcgtcatgatccgccgcacgccttgcaggagtggacgtccgtcaaagtacgtgcacgcgtgtgtttttactgtgtatagacggacggatgcatgtcttgctagctagcttgtaggcagcgtgtggcaacgaactgatatttcttctggtccgcgcagaacctatacgatgaaatgacggagcggattgactccgctgctcggacgggcaatgttcctgaagaaactagagcgaaacacaaagggttttctgagtgggatacgggtattacctcaaaagaccaccacccgatcgttcaggtatatatatcattattcccgtccctctatatttctccagacttttttgtattataaaaatatagatgatgttttcttttgctagattctgatagatgggaaagacaaggctgtagctgacaacgaaggcaatgtgctgccgacgctggtgtacgtggcacgagagaagaggcctcagtaccaccacaacttcaaagccggggcgatgaacgctctggtatgattcattcattcgcaccagctggtagcttagcatgcagggacattgtttgcttaacatatttataatatctgtgcgagatcgccgccaatttgaactgcagatccgagtatcgtccgtgataagcaacagccctatcatcctgaacgtggactgcgacatgtattccaacaacagcgacacgatcagagacgcgctgtgcttcttcctcgacgaagaaacgggccacaggatcgcgttcgtgcagtaccctcagaactacaacaacctcaccaagaacaacatatacggcaactccctcaatgtcatcaaccaggttagtactgtcaagtactgaaattatatatgcatgcctcttgacagcgacactgacaatgttgggcggcgctgaatcatcacaaggtggagctgagcggcctggacgcttggggcggcccgctgtacatcggcacgggatgcttccataggagggagaccctgtgcggcaggaggttcaccgaggactacaaggaagactgggacagaggaaccaaggagcagcagcagcaccgccaccgcgtcgacggcgagaccgaagcgaaggccaagtcgctagcgacctgcgcctacgagcacgacgacgacacgcggtggggagacgaggtggggctcaagtacggctgctcggtggaggacgtcatcacggggctggcgatacactgcagagggtgggagtcggtgtacagcaaccccgcgagagcggcgttcgtcggcgtcgcgccgaccacgctcgcccagaccatactgcagcacaagcggtggagcgagggcaacttcggcatcttcgtttccaggtactgccccttcgtctttggacgacggggcaaaaccaggttgccgcaccagatgggctactccatctacgggctatgggcgcccaactcgctgcctacgctgtactacgctgtcgtcccttcgctgtgcctgctcaagggcacccctctgttccctgaggtatgcatgtcgtacgtgtgattcaatggcattgaagcatatatatgtgctctctcttgagtcttgactgtgtgtgtgtgtttgtttcatcagctcacgagtccgtggatcgcgcctttcgtctacgtcgcggtcgccaagaacgtctacagcgcgtgggaggcgctgtggtgcggagacacgctgagagggtggtggaacgggcagaggatgtggctggtccggagaacgacctcgtacctctacggcttcgtcgacaccgtcagggactcgctggggctgtccaagatgggcttcgtggtgtcgtccaaggtgagcgacgaggacgaggccaagaggtacgagcaggagatgatggagttcgggacggcgtcgccggagtacgtgatcgtcgcggccgtcgcgctgctcaacctcgtgtgcctggcagggatggcggcggcactggatgtgttcttcgtccaggtcgctctctgcggggtgctggtgctcctcaacgtcccggtctatgaagccatgttcgtcaggaaggacagggggaggatgccgttcccgatcacgctagcctccgttggctttgtgacgctggccctcattgtgccattcttttgactttgaggtgctaataatacgtgtacgggcacacgcacgttcgcatgtatgacgattatgggcaacaggcgtgtaataccactaatacctattaaacactccagtctccaagtgatccattgctacacgtgtgttcctcctgttctctatatgcatgagctgctgatggtgatacgatactgtcagatactgcaataagacgccaaacaagataagcatcggcaatcgagtggatcctcacaaccacagtggacgctatgcaggtctttggaggcagttgctatgcacactttgattggcgcttcacaaatagactttcgttatgatggcgtcttttagtccctttagtcgttggttggtgaattgcaataggcgttctaatttcctgttgggtgtgtctgtttgtagatggtagtgtcatagttttatactcggtcctaacttcctttgaagggaaggccgtaattgtttcttttatctaaaaaaaaggcatcggagatataagatcgaaagacgtaagggtgggcaatcggagggggatgaacgaatgtttacggaagctcaaaaatatttataacgccacctcccaattgcaaagccaaattaattcacgtttctagctagaaaccggacagaactctgcttctttggtgaaaatgaggggactgtctatacacacgtgtgtttttagctgaaaaaacacacagattttttcaatgaaaatctgtgtgttttgcaaacacaaaacacacacatctcatatccactagatcaagatctaatggccataataaacacacagatttttctctctaaaaatagataaaaaacctaatatacaaaaaaaacaatacaagttgtttctgttaatatttaacatgttgtcttgtacttttttgttgttcaacaaaaatagatgtagaacaacaaaaatgacctctaacacagaaaacactgttatgtgctcagataacagtagttgcctgcatgaggccaaacacttctgaaaaatttgctactcagacacaaatctagaaaaaaatgatgaagcaaaaactcacaaaacagatcatacatcacacgaaaatagcgtacattgcaagggaagtggatacttgccacttcaactttttcagcttcatgatccctaggcttttctccacctagcaaagacaaaaaaatatataacattgaaaaaaactaaaacaaatcataaagtcaaaaaacatgatagacattacaatgatgcgaacgaaaatca

SEQ ID NO: 8 - DNA, 7274 bp, Zea mays
  misc_difference 1..446: note = n is a, c, g, or t
  source 1..7274: mol_type = unassigned DNA
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatcatcataatgttaatttatggaattaaaataatacggaaaatgcctatatttctaacaaaatatggtattagaaagtacatattgtattaatatttactataagtttcagcagattgagattgtatactctagataacgatgtttactgtcttcaacatatcatgtacatgatcatataaaatactatactattctacataataaataattataaacagtagagtttgaaatagaaaatcggtgaagacagccttacgctgacactgtcacttacactgaacacctcagtgcacgtgccgtctctgcaacgattagctgcatcggtcgctagccgcccctgtcggcgtacgtatcggcagcgagccaatgacacacgatccatcggctttatggggctgtttggttcatgactaaatgtgccacactttgtctaaggttagtcgttcgaattgaagaactaaccttaggcacaaaagttaggcaaagtgtgacaagttagccatcaaaccaaacaggccctatatacgccacccgctgctgctctccggtcctcggctcatcagtattattattattattattattcggctcctgctcatcagctgcagcagtcgtgctccggaccggagaagtcgaaatggaggagaggctgttcgccacggagaagcacggtggccgggcgctctacaggctccacgccgtcacggtgttcctggggatatgcctggtgctctgctacagggcgacgcacgtcccggctgccggctccggcggcagggcggcgtggctggggatgctcgcggcggagctctggttcggcttctactgggtcatcacgcagtccgtgcgctggtgccccatccgccgccgcaccttccacgacaggctcgccgccaggtttgccacttgacgaccttcttcgtgtgcatgcacatatacaaactattatccttgttgcacgtccggtcatttttcaaaggaaggctaatatgagttactaatgaggctaactggtggtcatatatagtccacactaactctctctatatattagcttaatacatgtaagtatcacacggtcacacacccctataagtacaagtgcatatgagcatcttatcttaaatcagtgctctatcttagaataatatagagcacaatcataaaaaacagttcatagaataatatagagcacaatcataaaaaacagttcaacaacatcttatgttacaaatatttatataaaaaaaatataggacacgctataaagtgacccaaatataacacgtctcatattagtgtcatattcttattttctatatcattatcaacattttcttttctaataatgcaatttacttcctcaaatacatgatatagagcttaacgattgtagctcaatttattttcagtgtcctgaacactttggatgatgtagtatttaatttttgggaaattattttgagacacctattcggagatgctctgcacatacactctcgtttcttgagggtttaaacccgtgacaatgcgtgaccccaaaccgtttctcttatactccctccgttttcaaattaaaattcgttttatttaattaatgggtttatataatatttggtatatttgtctagatctatcttcgaatacttaatatagatataaaaatcaagacctaaaccaactactattttagaatggatgaggagtatatattactaaaaaaaaattgaatttgaaacgatggttcacaaggtgagccgtttctacagctgggttcggaatgtggactgtggagtcctactctttcattccagtggcagctagaactagaagtttgcgacagccgacaggctgtgggggcaaacaaatctaagtatgaaatgaaaccgtgcgtgctacagactgcagaccacaggcacgcaggaggagcagataacagatagactcgtggggatgcatcagctgattgggggtccattcaggtccttctgcagcatatataagcgccgcgtgcagccaaattaaaacgatggatatggcatgggttcttctgactgcaccatgcctgtctgttttgttctactgactaactacagtttcccctctcgcgctgcctaagacgtcgaggccaaggccactgtgtagctagctcgcgagatttgttactgcagatggcagttgggccacgtgggctctatcgcacgccttggtgtgggggttgtcgttcagcgctacggctgtcgacgacggatgcgtttcaatgatgccgtcggcgacggtggtgctggtggcgtgtgtggctgtggtgtcacgggcttctaattctagcgccagttgcaccaatgcttttagggctcgtttggaacgtaggattgcccccccactatttcatgtttttttcctacgaaaaagaactgatttcagtgacattcctgcatctaaaatttccgcattccaaatgatacattagatttgtctccatgcggcccacaccataggatgtgacttgaagatggtgtccttgggcgcatgttctctggatccatttattttcgaggaattaaaatttattcaataaagtgatctatttgagttattggaatttaacattttatcactttttcagatataagactattttaaatttatgtggtggaggatgtgaaatgatattatatatcactagaatatgtttctactctgtaacttacacggcacacttcaactcattttctctaccgcaaaaatgtagcacataaaaacatttaacatcttgatgataataatatataaatatattttgaataaaattgtattagtctaaattagtatcgttagaatggaattcaattccaaggatctacctttgtagtaatgtgcgtcccccttcgatctccgttggtccgcatgaaactgaagctaattaggaaagtccaagacaccgtccatggtggtgtgtgcgcgtcctagtaatgcctgcgaggaggttggctgctaaacccagccagagcctcctatcatactgtccagtccaggcacaaaatatcctttggtgatgcacaagtcgggggtcacgaacatggctctctgcagggttcaggctcggacacattttggctgatactgcttcgttgattgtcggagcaggttcggagaacggctcccctgcgtggacatcttcgtgtgcacagcggacccgcggtcggagccgccgagccttgtcgtggccacggtcctgtcggtgatggcgtacaactacccgcccgcgaagctcaacgtgtacctctccgacgacggcggctccatcctcaccttctacgctatgtgggaggcctccgccttcgccaagcactggctcccgttctgcaggaggtacggcgtcgagccacggtcgccggccgcttacttcgcccagtcagatgagaagcctcgtcatgatccgccgcacgccttgcaggagtggacgtccgtcaaagtacgtgcacgcgtttgtttttactgtgtatagacggacggatgcatgtctagctagctagcttgtaggcagcgtgcacgtggcaacgaactgatatttcttctggtccgcgcagaacctatacgatgaaatgacggagcggattgactccgctgctaggacgggcaatgttcctgaagaaactagagcgaaacacaaagggttttctgagtgggatacgggtattacctcaaaagaccaccacccgatcgttcaggtatatatatcattattcccgtccctctatatttctccagacttttttgtattataaaaatatagatgatgttttcttttgctagattctgatagatgggaaagacaaggctgtagctgacaacgaaggcaatgtgctgccgacgctggtgtacgtggcacgagagaagaggcctcagtaccaccacaacttcaaagccggggcgatgaacgctctggtatgattcattcattcgcaccagctggtagcttagcatgcagggacattgtttgcttaacatatttataatatctgtgcgagatcgccgccaatttgaactgcagatccgagtatcgtccgtgataagcaacagccctatcatcctgaacgtggactgcgacatgtactccaacaacagcgacacgatcagagacgcgctgtgcttcttcctcgacgaagaaacgggccacaggatcgcgttcgtgcagtaccctcagaactacaacaacctcaccaagaacaacatatacggcaactccctcaatgtcatcaaccaggttagtgtcgagtactgaaattatatatgcatgcctcttgacagcgacactgacactgacactgttgggcggcgctgaatcatcacaaggtggagctgagcggcctggacgcttggggcggcccgctgtacatcggcacgggatgcttccataggagggagaccctgtgcggcaggaggttcaccgaggactacaaggaagactgggacagaggaaccaaggagcagcagcagcagcaccgcgtcgacggcgagaccgaagcgaaggccaagtcgctagcgacctgcgcctacgagcacgacgacgaggacacgcggtggggagacgaggtggggctcaagtacggctgctcggtggaggacgtcatcacggggctggcgatacactgcagagggtgggagtcggtgtacagcaaccccgcgagagcggcgttcgtcggcgtcgcgccgaccacgctcgcgcagaccatactgcagcacaagcggtggagcgagggcaacttcggcatcttcgtttccaggtactgccccttcgtctttggacgacggggcaaaaccaggttgccgcaccagatgggctactccatctacgggctatgggcgcccaactcgctgcctacgctgtactacgctgtcgtcccttcgctgtgcctgctcaagggcacccctctgttccctgaggtatatatgcatgtcgtacgtgtgattcaatggcattgaagcatatatatgtgctctctcttgagtcttgactgtgtgtgtgtgtttgtttcatcagctcacgagtccgtggatcgcgcctttcgtctacgtcgcggtcgccaagaacgtctacagcgcgtgggaggcgctgtggtgcggagacacgctgagagggtggtggaacgggcagaggatgtggctggtccggagaacgacctcgtacctctacggcttcgtcgacaccgtcagggactcgctggggctgtccaagatgggcttcgtggtgtcgtccaaggtgagcgacgaggacgaggccaagaggtacgagcaggagatgatggagttcgggacggcgtcgccggagtacgtgatcgtcgcggccgtcgcgctgctcaacctcgtgtgcctggcagggatggcggcggcactggatgtgttcttcgtccaggtcgctctctgcggggtgctggtgctcctcaacgtcccggtctatgaagccatgttcgtcaggaaggacagggggaggatgccgttcccgatcacgctagcctccgttggctttgtgacgctggccctcattgtgccattcttttgactttgaggtgctaataatacgtgtacgggcacacgcacgttcgcatgtatgacgattatgggcaacaggcgtgtaataccactaatacctattaaacactccagtctccaagtgatccattgctacacgtgtgttcctcctgttctctatatgcatgagctgctgatggtgatacgatactgtcagatactgcaataagacgccaaacaagataagcatcggcaatcgagtggatcctcacaaccacagtggacgctatgcaggtctttggaggcagttgctatgcacactttgattggcgcttcacaaatagactttcgttatgatggcgtcttttagtccctttagtcgttggttggtgaattgcaataggcgttctaatttcctgttgggtgtgtctgtttgtagatggtagtgtcatagttttatactcggtcctaacttcctttgaagggaaggccgtaattgtttcttttatctaaaaaaaaggcatcggagatataagatcgaaagacgtaagggtgggcaatcggagggggatgaacgaatgtttacggaagctcaaaaatatttataacgccacctcccaattgcaaagccaaattaattcacgtttctagctagaaaccggacagaactctgcttctttggtgaaaatgaggggactgtctatacacacgtgtgtttttagctgaaaaaacacacagattttttcaatgaaaatctgtgtgttttgcaaacacaaaacacacacatctcatatccactagatcaagatctaatggccataataaacacacagatttttctctctaaaaatagataaaaaacctaatatacaaaaaaaacaatacaagttgtttctgttaatatttaacatgttgtcttgtacttttttgttgttcaacaaaaatagatgtagaacaacaaaaatgacctctaacacagaaaacactgttatgtgctcagataacagtagttgcctgcatgaggccaaacacttctgaaaaatttgctactcagacacaaatctagaaaaaaatgatgaagcaaaaactcacaaaacagatcatacatcacacgaaaatagcgtacattgcaagggaagtggatacttgccacttcaactttttcagcttcatgatccctaggcttttctccacctagcaaagacaaaaaaatatataacattgaaaaaaactaaaacaaatcataaagtcaaaaaacatgatagacattacaatgatgcgaacgaaaatca

SEQ ID NO: 9 - DNA, 2941 bp, Zea mays
  source 1..2941: mol_type = unassigned DNA
accattcgaaagatccctccaggaaagatttttcttccctcctccgacgccccagcccaccaacacactctataaagcagccctcagtcacacacagaacgcacaagcgcaagccgggcaagaaaactccgcaggccagtctgcgagttggatggccttcttcttggccctcgtgtgcatcctcatcttgctagccatcgcctcctacgtccagtacactcgctggcaaaaggggaaaggccgcttcggcggccatgggaggtctgctcccttgaagctgcctcctggctccatgggctggccttaccttggcgagaccctccagctttactcccaggaccccagcttcttcttcgcttccaaacagaagaggttagtcgccgtaggcaactactactactcatgcgggcagcgtgttcgtccttcgttctggatccgcccccttgttcacaagctgctaatgattcgaacggaacgaccatgcatgccttgtgtgcaggtacggcgagatcttcaagacgcaccttctgggttgcccgtgcgtgatgctggcgagcccggaggcggcgcggttcgtgctggtgacgcaggcgcacctgttcaagccgacctacccgcggagcaaggagcgcatgatcgggccgtcggctctcttcttccaccagggcgactaccacctccgcctccgcaagctcgtccagggcgcgctcggccccgacgcgctgcgcgcgctcgttcctgaggtggaggccgccgtgcggtccactctcgcttcctgggacgccggccacgtcagaagcacgttccacgccatgaagacggtaaggaataataataatagtcaagcatgcatgcgcggccaattatataatgttggaatgaatcgggtgctgagaattaatacgattgtttgcttctgttgttacgtttcagctgtcgtttgatgtgggcatcgtgacgatcttcggcggccggctggacgagcggcgcaaggcggagctgaggaagaattactccgtcgtggagaaggggtacaactccttccccaacagcctgccggggacgctccattacaaggcgatgcaggtgagcacacacgcgacacggcatttacacaacccatccaacgcattacacgtacggtacgtctcgggcaacggcagtacgtactgccctgcccctggcacgcacgcatgcatgtgacgaaatcgctggacaccgtaccgtacgtacaccgtaggcgcggcggcggctgcacggcgtgctgtgcgacatcatgcgggagcgtcgtggccaggcccaggcggcgggcaccggcctgctgggctgcctgatgcggtcccggggcgacgacggcgcgccgctcctgagcgacgagcagatcgccgacaacgtcatcggcgtgctgttcgcggcgcaggacacgacggccagcgcgctcacctggatcgtcaagtacctccacgaccaccccaagctgctcgaggccgtccgggcggagcaggcggcggtccgcgaggccaccggcggcgggaggcagccgctggcgtgggcgcacacgaagagcatggcgctaacgcatagggtacgagcgtgcgtgctgggaaacgcaaaactggctctttattatttttttcttgtggtttcatccgtacgtcgcccgtccaggtgattttggagagcttaaggatggcgagcatcatctcgttcacgttcagggaggccgtggccgacgtggagtacaaaggtacgcacgcacgtgcgcgcaccacgaagagtagctagaggagcaacgagagtgctttgcttaattctgactcggattatgccgtgtagggttccttatccccaaggggtggaaggtgatgccgctcttcaggaacatccaccacagcccggactacttccaggatccacacaagttcgacccttctagattccaggtacgttacgtacagaagcatgggcctcaccgccgttagttgctgtgggacgacgacgacgtgactgaccggacgttgcgtattatgcaggtggcgccgcgtccgagcacgttcctgccgtttgggcacggcgtgcacgcgtgccccgggaacgagctggccaagctcgagatgctcgtcctcatccaccacctggtcaccggctacaggtgcgtccatctcctctcagatcctctccatatattcccgcttgtcctatagcttgtggaccaggatgacacatggctggctgctgccgctctccatggggctccggctctgatctctctccgtgcatgctccaaatctcctcctgtctgtatgtatgcctgtatcgatcatgtatatactcctgtaccataatctgtggggtcctcgaaatgtacgtcttcactagccccgctgtgctctccctcctatataaactgtggtgatcgactgctataacgacagtttactgatcttacactgagacactgattggcgtctctgcatgctttatttttaaatttgcaggtggcaaatcgttggatccagtgacgaggtcgagtacagcccgttccctgtgcccaagcacggcttgcctgtcagattatggagacaaaacaatccggtcgacagaaaggggcgtgagaccgacgacgatcatgtggagaggatatttatttagtttgactcttgagttaggcatgaatttaaccccaagctagctagagaagttttttttcccctttgaaattcttctttgctcgcctcttcctcctggatcaaattgcgttggaggagaagaaacggcagctttctctctttcgttttctttgcctgcttcaccgctacgataatggtgaaaatatgtaagctacgtggacatcaatgatccacagcatcgttgatatatataatatatagagaaaattctctgcacgatcaatgcaattttatccggtatcttatttacc

SEQ ID NO: 10 - DNA, 1910 bp, Zea mays
  source 1..1910: mol_type = unassigned DNA
accattcgaaagatccctccaggaaagatttttcttccctcctccgacgccccagcccaccaacacactctataaagcagccctcagtcacacacagaacgcacaagcgcaagccgggcaagaaaactccgcaggccagtctgcgagttggatggccttcttcttggccctcgtgtgcatcctcatcttgctagccatcgcctcctacgtccagtacactcgctggcaaaaggggaaaggccgcttcggcggccatgggaggtctgctcccttgaagctgcctcctggctccatgggctggccttaccttggcgagaccctccagctttactcccaggaccccagcttcttcttcgcttccaaacagaagaggtacggcgagatcttcaagacgcaccttctgggttgcccgtgcgtgatgctggcgagcccggaggcggcgcggttcgtgctggtgacgcaggcgcacctgttcaagccgacctacccgcggagcaaggagcgcatgatcgggccgtcggctctcttcttccaccagggcgactaccacctccgcctccgcaagctcgtccagggcgcgctcggccccgacgcgctgcgcgcgctcgttcctgaggtggaggccgccgtgcggtccactctcgcttcctgggacgccggccacgtcagaagcacgttccacgccatgaagacgctgtcgtttgatgtgggcatcgtgacgatcttcggcggccggctggacgagcggcgcaaggcggagctgaggaagaattactccgtcgtggagaaggggtacaactccttccccaacagcctgccggggacgctccattacaaggcgatgcaggcgcggcggcggctgcacggcgtgctgtgcgacatcatgcgggagcgtcgtggccaggcccaggcggcgggcaccggcctgctgggctgcctgatgcggtcccggggcgacgacggcgcgccgctcctgagcgacgagcagatcgccgacaacgtcatcggcgtgctgttcgcggcgcaggacacgacggccagcgcgctcacctggatcgtcaagtacctccacgaccaccccaagctgctcgaggccgtccgggcggagcaggcggcggtccgcgaggccaccggcggcgggaggcagccgctggcgtgggcgcacacgaagagcatggcgctaacgcatagggtgattttggagagcttaaggatggcgagcatcatctcgttcacgttcagggaggccgtggccgacgtggagtacaaagggttccttatccccaaggggtggaaggtgatgccgctcttcaggaacatccaccacagcccggactacttccaggatccacacaagttcgacccttctagattccaggtggcgccgcgtccgagcacgttcctgccgtttgggcacggcgtgcacgcgtgccccgggaacgagctggccaagctcgagatgctcgtcctcatccaccacctggtcaccggctacaggtggcaaatcgttggatccagtgacgaggtcgagtacagcccgttccctgtgcccaagcacggcttgcctgtcagattatggagacaaaacaatccggtcgacagaaaggggcgtgagaccgacgacgatcatgtggagaggatatttatttagtttgactcttgagttaggcatgaatttaaccccaagctagctagagaagttttttttcccctttgaaattcttctttgctcgcctcttcctcctggatcaaattgcgttggaggagaagaaacggcagctttctctctttcgttttctttgcctgcttcaccgctacgataatggtgaaaatatgtaagctacgtggacatcaatgatccacagcatcgttgatatatataatatatagagaaaattctctgcacgatc

SEQ ID NO: 11 - DNA, 1500 bp, synthetic construct
  misc_feature 1..1500: note = cDNA (transcript 1) of ZmAbh4 derived from B73
  source 1..1500: mol_type = other DNA
atggccttcttcttggccctcgtgtgcatcctcatcttgctagccatcgcctcctacgtccagtacactcgctggcaaaaggggaaaggccgcttcggcggccatgggaggtctgctcccttgaagctgcctcctggctccatgggctggccttaccttggcgagaccctccagctttactcccaggaccccagcttcttcttcgcttccaaacagaagaggtacggcgagatcttcaagacgcaccttctgggttgcccgtgcgtgatgctggcgagcccggaggcggcgcggttcgtgctggtgacgcaggcgcacctgttcaagccgacctacccgcggagcaaggagcgcatgatcgggccgtcggctctcttcttccaccagggcgactaccacctccgcctccgcaagctcgtccagggcgcgctcggccccgacgcgctgcgcgcgctcgttcctgaggtggaggccgccgtgcggtccactctcgcttcctgggacgccggccacgtcagaagcacgttccacgccatgaagacgctgtcgtttgatgtgggcatcgtgacgatcttcggcggccggctggacgagcggcgcaaggcggagctgaggaagaattactccgtcgtggagaaggggtacaactccttccccaacagcctgccggggacgctccattacaaggcgatgcaggcgcggcggcggctgcacggcgtgctgtgcgacatcatgcgggagcgtcgtggccaggcccaggcggcgggcaccggcctgctgggctgcctgatgcggtcccggggcgacgacggcgcgccgctcctgagcgacgagcagatcgccgacaacgtcatcggcgtgctgttcgcggcgcaggacacgacggccagcgcgctcacctggatcgtcaagtacctccacgaccaccccaagctgctcgaggccgtccgggcggagcaggcggcggtccgcgaggccaccggcggcgggaggcagccgctggcgtgggcgcacacgaagagcatggcgctaacgcatagggtgattttggagagcttaaggatggcgagcatcatctcgttcacgttcagggaggccgtggccgacgtggagtacaaagggttccttatccccaaggggtggaaggtgatgccgctcttcaggaacatccaccacagcccggactacttccaggatccacacaagttcgacccttctagattccaggtggcgccgcgtccgagcacgttcctgccgtttgggcacggcgtgcacgcgtgccccgggaacgagctggccaagctcgagatgctcgtcctcatccaccacctggtcaccggctacaggtggcaaatcgttggatccagtgacgaggtcgagtacagcccgttccctgtgcccaagcacggcttgcctgtcagattatggagacaaaacaatccggtcgacagaaaggggcgtgagaccgacgacgatcatgtggagaggatatttatttag

SEQ ID NO: 12 - protein, 499 aa, Zea mays
MAFFLALVCILILLAIASYVQYTRWQKGKGRFGGHGRSAPLKLPPGSMGWPYLGETLQLYSQDPSFFFASKQKRYGEIFKTHLLGCPCVMLASPEAARFVLVTQAHLFKPTYPRSKERMIGPSALFFHQGDYHLRLRKLVQGALGPDALRALVPEVEAAVRSTLASWDAGHVRSTFHAMKTLSFDVGIVTIFGGRLDERRKAELRKNYSVVEKGYNSFPNSLPGTLHYKAMQARRRLHGVLCDIMRERRGQAQAAGTGLLGCLMRSRGDDGAPLLSDEQIADNVIGVLFAAQDTTASALTWIVKYLHDHPKLLEAVRAEQAAVREATGGGRQPLAWAHTKSMALTHRVILESLRMASIISFTFREAVADVEYKGFLIPKGWKVMPLFRNIHHSPDYFQDPHKFDPSRFQVAPRPSTFLPFGHGVHACPGNELAKLEMLVLIHHLVTGYRWQIVGSSDEVEYSPFPVPKHGLPVRLWRQNNPVDRKGRETDDDHVERIFI

SEQ ID NO: 13 - DNA, 2059 bp, Zea mays
  source 1..2059: mol_type = unassigned DNA
accattcgaaagatccctccaggaaagatttttcttccctcctccgacgccccagcccaccaacacactctataaagcagccctcagtcacacacagaacgcacaagcgcaagccgggcaagaaaactccgcaggccagtctgcgagttggatggccttcttcttggccctcgtgtgcatcctcatcttgctagccatcgcctcctacgtccagtacactcgctggcaaaaggggaaaggccgcttcggcggccatgggaggtctgctcccttgaagctgcctcctggctccatgggctggccttaccttggcgagaccctccagctttactcccaggaccccagcttcttcttcgcttccaaacagaagaggtacggcgagatcttcaagacgcaccttctgggttgcccgtgcgtgatgctggcgagcccggaggcggcgcggttcgtgctggtgacgcaggcgcacctgttcaagccgacctacccgcggagcaaggagcgcatgatcgggccgtcggctctcttcttccaccagggcgactaccacctccgcctccgcaagctcgtccagggcgcgctcggccccgacgcgctgcgcgcgctcgttcctgaggtggaggccgccgtgcggtccactctcgcttcctgggacgccggccacgtcagaagcacgttccacgccatgaagacgctgtcgtttgatgtgggcatcgtgacgatcttcggcggccggctggacgagcggcgcaaggcggagctgaggaagaattactccgtcgtggagaaggggtacaactccttccccaacagcctgccggggacgctccattacaaggcgatgcaggcgcggcggcggctgcacggcgtgctgtgcgacatcatgcgggagcgtcgtggccaggcccaggcggcgggcaccggcctgctgggctgcctgatgcggtcccggggcgacgacggcgcgccgctcctgagcgacgagcagatcgccgacaacgtcatcggcgtgctgttcgcggcgcaggacacgacggccagcgcgctcacctggatcgtcaagtacctccacgaccaccccaagctgctcgaggccgtccgggcggagcaggcggcggtccgcgaggccaccggcggcgggaggcagccgctggcgtgggcgcacacgaagagcatggcgctaacgcatagggtgattttggagagcttaaggatggcgagcatcatctcgttcacgttcagggaggccgtggccgacgtggagtacaaagggttccttatccccaaggggtggaaggtgatgccgctcttcaggaacatccaccacagcccggactacttccaggatccacacaagttcgacccttctagattccaggtggcgccgcgtccgagcacgttcctgccgtttgggcacggcgtgcacgcgtgccccgggaacgagctggccaagctcgagatgctcgtcctcatccaccacctggtcaccggctacaggtgcgtccatctcctctcagatcctctccatatattcccgcttgtcctatagcttgtggaccaggatgacacatggctggctgctgccgctctccatggggctccggctctgatctctctccgtgcatgctccaaatctcctcctgtctgtggcaaatcgttggatccagtgacgaggtcgagtacagcccgttccctgtgcccaagcacggcttgcctgtcagattatggagacaaaacaatccggtcgacagaaaggggcgtgagaccgacgacgatcatgtggagaggatatttatttagtttgactcttgagttaggcatgaatttaaccccaagctagctagagaagttttttttcccctttgaaattcttctttgctcgcctcttcctcctggatcaaattgcgttggaggagaagaaacggcagctttctctctttcgttttctttgcctgcttcaccgctacgataatggtgaaaatatgtaagctacgtggacatcaatgatccacagcatcgttgatatatataatatatagagaaaattctctgcacgatc

SEQ ID NO: 14 - DNA, 1398 bp, synthetic construct
  misc_feature 1..1398: note = cDNA (transcript 2) of ZmAbh4 derived from B73
  source 1..1398: mol_type = other DNA
atggccttcttcttggccctcgtgtgcatcctcatcttgctagccatcgcctcctacgtccagtacactcgctggcaaaaggggaaaggccgcttcggcggccatgggaggtctgctcccttgaagctgcctcctggctccatgggctggccttaccttggcgagaccctccagctttactcccaggaccccagcttcttcttcgcttccaaacagaagaggtacggcgagatcttcaagacgcaccttctgggttgcccgtgcgtgatgctggcgagcccggaggcggcgcggttcgtgctggtgacgcaggcgcacctgttcaagccgacctacccgcggagcaaggagcgcatgatcgggccgtcggctctcttcttccaccagggcgactaccacctccgcctccgcaagctcgtccagggcgcgctcggccccgacgcgctgcgcgcgctcgttcctgaggtggaggccgccgtgcggtccactctcgcttcctgggacgccggccacgtcagaagcacgttccacgccatgaagacgctgtcgtttgatgtgggcatcgtgacgatcttcggcggccggctggacgagcggcgcaaggcggagctgaggaagaattactccgtcgtggagaaggggtacaactccttccccaacagcctgccggggacgctccattacaaggcgatgcaggcgcggcggcggctgcacggcgtgctgtgcgacatcatgcgggagcgtcgtggccaggcccaggcggcgggcaccggcctgctgggctgcctgatgcggtcccggggcgacgacggcgcgccgctcctgagcgacgagcagatcgccgacaacgtcatcggcgtgctgttcgcggcgcaggacacgacggccagcgcgctcacctggatcgtcaagtacctccacgaccaccccaagctgctcgaggccgtccgggcggagcaggcggcggtccgcgaggccaccggcggcgggaggcagccgctggcgtgggcgcacacgaagagcatggcgctaacgcatagggtgattttggagagcttaaggatggcgagcatcatctcgttcacgttcagggaggccgtggccgacgtggagtacaaagggttccttatccccaaggggtggaaggtgatgccgctcttcaggaacatccaccacagcccggactacttccaggatccacacaagttcgacccttctagattccaggtggcgccgcgtccgagcacgttcctgccgtttgggcacggcgtgcacgcgtgccccgggaacgagctggccaagctcgagatgctcgtcctcatccaccacctggtcaccggctacaggtgcgtccatctcctctcagatcctctccatatattcccgcttgtcctatag

SEQ ID NO: 15 - protein, 465 aa, Zea mays
MAFFLALVCILILLAIASYVQYTRWQKGKGRFGGHGRSAPLKLPPGSMGWPYLGETLQLYSQDPSFFFASKQKRYGEIFKTHLLGCPCVMLASPEAARFVLVTQAHLFKPTYPRSKERMIGPSALFFHQGDYHLRLRKLVQGALGPDALRALVPEVEAAVRSTLASWDAGHVRSTFHAMKTLSFDVGIVTIFGGRLDERRKAELRKNYSVVEKGYNSFPNSLPGTLHYKAMQARRRLHGVLCDIMRERRGQAQAAGTGLLGCLMRSRGDDGAPLLSDEQIADNVIGVLFAAQDTTASALTWIVKYLHDHPKLLEAVRAEQAAVREATGGGRQPLAWAHTKSMALTHRVILESLRMASIISFTFREAVADVEYKGFLIPKGWKVMPLFRNIHHSPDYFQDPHKFDPSRFQVAPRPSTFLPFGHGVHACPGNELAKLEMLVLIHHLVTGYRCVHLLSDPLHIFPLVL

SEQ ID NO: 16 - DNA, 2286 bp, Zea mays
  source 1..2286: mol_type = unassigned DNA
accattcgaaagatccctccaggaaagatttttcttccctcctccgacgccccagcccaccaacacactctataaagcagccctcagtcacacacagaacgcacaagcgcaagccgggcaagaaaactccgcaggccagtctgcgagttggatggccttcttcttggccctcgtgtgcatcctcatcttgctagccatcgcctcctacgtccagtacactcgctggcaaaaggggaaaggccgcttcggcggccatgggaggtctgctcccttgaagctgcctcctggctccatgggctggccttaccttggcgagaccctccagctttactcccaggaccccagcttcttcttcgcttccaaacagaagaggtacggcgagatcttcaagacgcaccttctgggttgcccgtgcgtgatgctggcgagcccggaggcggcgcggttcgtgctggtgacgcaggcgcacctgttcaagccgacctacccgcggagcaaggagcgcatgatcgggccgtcggctctcttcttccaccagggcgactaccacctccgcctccgcaagctcgtccagggcgcgctcggccccgacgcgctgcgcgcgctcgttcctgaggtggaggccgccgtgcggtccactctcgcttcctgggacgccggccacgtcagaagcacgttccacgccatgaagacgctgtcgtttgatgtgggcatcgtgacgatcttcggcggccggctggacgagcggcgcaaggcggagctgaggaagaattactccgtcgtggagaaggggtacaactccttccccaacagcctgccggggacgctccattacaaggcgatgcaggcgcggcggcggctgcacggcgtgctgtgcgacatcatgcgggagcgtcgtggccaggcccaggcggcgggcaccggcctgctgggctgcctgatgcggtcccggggcgacgacggcgcgccgctcctgagcgacgagcagatcgccgacaacgtcatcggcgtgctgttcgcggcgcaggacacgacggccagcgcgctcacctggatcgtcaagtacctccacgaccaccccaagctgctcgaggccgtccgggcggagcaggcggcggtccgcgaggccaccggcggcgggaggcagccgctggcgtgggcgcacacgaagagcatggcgctaacgcatagggtgattttggagagcttaaggatggcgagcatcatctcgttcacgttcagggaggccgtggccgacgtggagtacaaagggttccttatccccaaggggtggaaggtgatgccgctcttcaggaacatccaccacagcccggactacttccaggatccacacaagttcgacccttctagattccaggtggcgccgcgtccgagcacgttcctgccgtttgggcacggcgtgcacgcgtgccccgggaacgagctggccaagctcgagatgctcgtcctcatccaccacctggtcaccggctacaggtgcgtccatctcctctcagatcctctccatatattcccgcttgtcctatagcttgtggaccaggatgacacatggctggctgctgccgctctccatggggctccggctctgatctctctccgtgcatgctccaaatctcctcctgtctgtatgtatgcctgtatcgatcatgtatatactcctgtaccataatctgtggggtcctcgaaatgtacgtcttcactagccccgctgtgctctccctcctatataaactgtggtgatcgactgctataacgacagtttactgatcttacactgagacactgattggcgtctctgcatgctttatttttaaatttgcaggtggcaaatcgttggatccagtgacgaggtcgagtacagcccgttccctgtgcccaagcacggcttgcctgtcagattatggagacaaaacaatccggtcgacagaaaggggcgtgagaccgacgacgatcatgtggagaggatatttatttagtttgactcttgagttaggcatgaatttaaccccaagctagctagagaagttttttttcccctttgaaattcttctttgctcgcctcttcctcctggatcaaattgcgttggaggagaagaaacggcagctttctctctttcgttttctttgcctgcttcaccgctacgataatggtgaaaatatgtaagctacgtggacatcaatgatccacagcatcgttgatatatataatatatagagaaaattctctgcacgatcaatgcaattttatccggtatcttatttacc

SEQ ID NO: 17 - DNA, 1398 bp, synthetic construct; identical to SEQ ID NO: 14
  misc_feature 1..1398: note = cDNA (transcript 3) of ZmAbh4 derived from B73
  source 1..1398: mol_type = other DNA

SEQ ID NO: 18 - DNA, 3503 bp, Zea mays
  misc_difference 1719..2218: note = n is a, c, g, or t
  source 1..3503: mol_type = unassigned DNA
accattcgaaagatccctccaggaaagatttttcttccctcctccgacgccccagcccaccaacacactctataaagcagcccccagtcacacacagaacgcacaagcgcaagccgggcaagaaaactccgcaggccagtctgcgagttggatggccttcttcttggccctcgtgtgcatcctcgtcttcctagccaccgcctcctacgtccagtacactcgctggcagaaggggaaaggccgcttcggcggccatgggaggtctgctcccttgaagctgcctcctggctccatgggctggccttaccttggcgagaccctccagctttactcccaggaccccagcgtcttcttcgcttccaaacagaagaggttagtcgccgtaggcaactactagtcatgcgggcagcgtgttcgtccttcgttctcgatccgcccccttgttcacaagctgctaatgattcgaacggaacgaccgtgcatgccttgtgtgcaggtacggcgagatcttcaagacgcaccttctgggctgcccgtgcgtgatgctggcgagcccggaggcggcgcggttcgtgctggtgacgcaggcgcacctgttcaagccgacctacccgcggagcaaggagcgcatgatcgggccgtcggctctcttcttccaccagggcgactaccacctccgcctccgcaagctcgtccagggcgcgctcggccccgacgcgctgcgcgcgctcgttcctgaggtggaggccgccgtgcggtccactctcgcttcctgggacgccggccacgtcagaagcacgttccacgccatgaagacggtaaggaataataataatagtcaagcatgcatgcgcggccaattatataatgttggaatgaatcgggtgctgagaattaatacgattgtttgcttctgttgttacgtttcagctgtcgtttgatgtgggcatcgtgacgatcttcggcggccggctggacgagcggcgcaaggcggagctgaggaagaattactccgtcgtggagaaggggtacaactccttccccaacagcctgccggggacgctccattacaaggcgatgcaggtgagcacacacgcgacacggcatttacacaacccatccaacgcattacacgtacggtacgtctcgggcaacggcagtacgtactgccctgcccctggcacgcacgcatgcatgtgacgaaatcgctggacaccgtaccgtacgtacaccgtaggcgcggcggcggctgcacggcgtgctgtgcgacatcatgcgggagcgtcgaggccaggcccaggcgggcaccggcctgctgggctgcctgatgcggtcccggggcgacgacggcgcgccgctgctgagcgacgagcagatcgccgacaacgtcatcggcgtgctgttcgcggcgcaggacacgacggccagcgcgctcacctggatcgtcaagtacctccacgaccaccccaagctgctcgaggccgtccgggcggagcaggcggcggtccgcgaggccaccggcggcgggaggcagccgctggcgtgggcgcacacgaagagcatggcgctaacgcatagggtacgagcgtgcgtgctgggaaacgcaaaactggctcttcattatttttttcttgtggtttcatccgtacgtcgcccgtccaggtgattttggagagcttaaggatggcgagcatcatctcgttcacgttcagggaggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgagcatcatctcgttcacgttcagggaggccgtggccgacgtggagtacaaaggtacgcacgtacgtgcgcgcaccacgaagagtagctagaggagcaacgagagttggtttgcttaattctgactcggattattccatgtatcgatctccttccttggcgtacgtgtagggttccttatccccaaggggtggaaggtgatgcctctcttcaggaacatccaccacagccctgactacttccaggatccacacaagttcgacccttctagattccaggtacgttacgtacgtacagaagcatgggcctcaccgccgttagttgctgtgggacgacgacgacgtgactgaccggacgttgcgtattatgcaggtggcgccgcgtccgagcacgttcctgccgtttgggcacggcgtgcacgcgtgccccgggaacgagctggccaagctcgagatgctcgtcctcatccaccacctggtcaccggctacaggtgcgtccatctcctctcagatcctctccatatattccccgcttgtcctatagcttgtggaccaggatgacacatggctggctgctgccgctctccatggggctccggctctctctctccgtgcatgctccaaatctcctcctgtctgtatgtatgcctgtatcgatcatgtatatactcctgtaccataatctgtggggtcctcgaaatgtacgtcttcactagccccgctgtgctctccttcctcctatatatactatatatatgatgagcatggcgatcgactgctataacgacggtttactgatcttacactgaggcactgattggcgtccctgcatgctttatttttaaatttgcaggtggcaaatcgttggatccagtgacgaggtcgagtacagcccgttccctgtgcccaagcacggcttgcctgtcagattatggagacaaaacaatccggtcgtcgacagaaaggggcgtgagaccgacgacgatgatgtggaggatattttagtttgactcttgagttaggcatgaatttaaccccaagctagctagagaagttttttttccctttgaaattcttctttgctcgccttcctcctggatcaaattacgttggaggacaagaaacggcagctttctctttcgttttctttgcctgcttcaccgcgacgataatggtgaaaatatgtaagctacgtggacatcaatgatccacagcatcgttgatatatataatatagagagaaaattctctgcacggtcaatgcagttttatccggtatcttatt

SEQ ID NO: 19 - DNA, 1701 bp, Zea mays
  source 1..1701: mol_type = unassigned DNA
gcgctcgctcgacctccaccattcgaaagatccctccaggaaagatttttcttccctcctccgacgccccagcccaccaacacactctataaagcagcccccagtcacacacagaacgcacaagcgcaagccgggcaagaaaactccgcaggccagtctgcgagttggatggccttcttcttggccctcgtgtgcatcctcgtcttcctagccaccgcctcctacgtccagtacactcgctggcagaaggggaaaggccgcttcggcggccatgggaggtctgctcccttgaagctgcctcctggctccatgggctggccttaccttggcgagaccctccagctttactcccaggaccccagcgtcttcttcgcttccaaacagaagaggtacggcgagatcttcaagacgcaccttctgggctgcccgtgcgtgatgctggcgagcccggaggcggcgcggttcgtgctggtgacgcaggcgcacctgttcaagccgacctacccgcggagcaaggagcgcatgatcgggccgtcggctctcttcttccaccagggcgactaccacctccgcctccgcaagctcgtccagggcgcgctcggccccgacgcgctgcgcgcgctcgttcctgaggtggaggccgccgtgcggtccactctcgcttcctgggacgccggccacgtcagaagcacgttccacgccatgaagacgctgtcgtttgatgtgggcatcgtgacgatcttcggcggccggctggacgagcggcgcaaggcggagctgaggaagaattactccgtcgtggagaaggggtacaactccttccccaacagcctgccggggacgctccattacaaggcgatgcaggcgcggcggcggctgcacggcgtgctgtgcgacatcatgcgggagcgtcgaggccaggcccaggcgggcaccggcctgctgggctgcctgatgcggtcccggggcgacgacggcgcgccgctgctgagcgacgagcagatcgccgacaacgtcatcggcgtgctgttcgcggcgcaggacacgacggccagcgcgctcacctggatcgtcaagtacctccacgaccaccccaagctgctcgaggccgtccgggcggagcaggcggcggtccgcgaggccaccggcggcgggaggcagccgctggcgtgggcgcacacgaagagcatggcgctaacgcatagggtacgagcgtgcgtgctgggaaacgcaaaactggctcttcattatttttttcttgtggtttcatccgtacgtcgcccgtccagggaggccgtggccgacgtggagtacaaagggttccttatccccaaggggtggaaggtgatgcctctcttcaggaacatccaccacagccctgactacttccaggatccacacaagttcgacccttctagattccaggtggcgccgcgtccgagcacgttcctgccgtttgggcacggcgtgcacgcgtgccccgggaacgagctggccaagctcgagatgctcgtcctcatccaccacctggtcaccggctacaggtggcaaatcgttggatccagtgacgaggtcgagtacagcccgttccctgtgcccaagcacggcttgcctgtcagattatggagacaaaacaatccggtcgtcgacagaaaggggcgtgagaccgacgacgatgatgtggaggatattttagtttga

SEQ ID NO: 20 - DNA, 1533 bp, synthetic construct
  misc_feature 1..1533: note = cDNA of ZmAbh4 derived from PH207
  source 1..1533: mol_type = other DNA
atggccttcttcttggccctcgtgtgcatcctcgtcttcctagccaccgcctcctacgtccagtacactcgctggcagaaggggaaaggccgcttcggcggccatgggaggtctgctcccttgaagctgcctcctggctccatgggctggccttaccttggcgagaccctccagctttactcccaggaccccagcgtcttcttcgcttccaaacagaagaggtacggcgagatcttcaagacgcaccttctgggctgcccgtgcgtgatgctggcgagcccggaggcggcgcggttcgtgctggtgacgcaggcgcacctgttcaagccgacctacccgcggagcaaggagcgcatgatcgggccgtcggctctcttcttccaccagggcgactaccacctccgcctccgcaagctcgtccagggcgcgctcggccccgacgcgctgcgcgcgctcgttcctgaggtggaggccgccgtgcggtccactctcgcttcctgggacgccggccacgtcagaagcacgttccacgccatgaagacgctgtcgtttgatgtgggcatcgtgacgatcttcggcggccggctggacgagcggcgcaaggcggagctgaggaagaattactccgtcgtggagaaggggtacaactccttccccaacagcctgccggggacgctccattacaaggcgatgcaggcgcggcggcggctgcacggcgtgctgtgcgacatcatgcgggagcgtcgaggccaggcccaggcgggcaccggcctgctgggctgcctgatgcggtcccggggcgacgacggcgcgccgctgctgagcgacgagcagatcgccgacaacgtcatcggcgtgctgttcgcggcgcaggacacgacggccagcgcgctcacctggatcgtcaagtacctccacgaccaccccaagctgctcgaggccgtccgggcggagcaggcggcggtccgcgaggccaccggcggcgggaggcagccgctggcgtgggcgcacacgaagagcatggcgctaacgcatagggtacgagcgtgcgtgctgggaaacgcaaaactggctcttcattatttttttcttgtggtttcatccgtacgtcgcccgtccagggaggccgtggccgacgtggagtacaaagggttccttatccccaaggggtggaaggtgatgcctctcttcaggaacatccaccacagccctgactacttccaggatccacacaagttcgacccttctagattccaggtggcgccgcgtccgagcacgttcctgccgtttgggcacggcgtgcacgcgtgccccgggaacgagctggccaagctcgagatgctcgtcctcatccaccacctggtcaccggctacaggtggcaaatcgttggatccagtgacgaggtcgagtacagcccgttccctgtgcccaagcacggcttgcctgtcagattatggagacaaaacaatccggtcgtcgacagaaaggggcgtgagaccgacgacgatgatgtggaggatattttagtttga

SEQ ID NO: 21 - protein, 510 aa, Zea mays
MAFFLALVCILVFLATASYVQYTRWQKGKGRFGGHGRSAPLKLPPGSMGWPYLGETLQLYSQDPSVFFASKQKRYGEIFKTHLLGCPCVMLASPEAARFVLVTQAHLFKPTYPRSKERMIGPSALFFHQGDYHLRLRKLVQGALGPDALRALVPEVEAAVRSTLASWDAGHVRSTFHAMKTLSFDVGIVTIFGGRLDERRKAELRKNYSVVEKGYNSFPNSLPGTLHYKAMQARRRLHGVLCDIMRERRGQAQAGTGLLGCLMRSRGDDGAPLLSDEQIADNVIGVLFAAQDTTASALTWIVKYLHDHPKLLEAVRAEQAAVREATGGGRQPLAWAHTKSMALTHRVRACVLGNAKLALHYFFLVVSSVRRPSREAVADVEYKGFLIPKGWKVMPLFRNIHHSPDYFQDPHKFDPSRFQVAPRPSTFLPFGHGVHACPGNELAKLEMLVLIHHLVTGYRWQIVGSSDEVEYSPFPVPKHGLPVRLWRQNNPVVDRKGRETDDDDVEDILV

SEQ ID NO: 22 - DNA, 4941 bp, Zea mays
  source 1..4941: mol_type = unassigned DNA
ctaccgtcggccagtagactacctataccattttctatttcaaactctactctataaatagagcaatttacagtataaaacaatattttgcatgaccatttacacgacctttcagagatgatctaaggagataaaagatttgcatgagcacaagaagagggaataggagaagaacgaagaatatgagtatttggtgtacataggtctgaagcaagatgaatggagaagggtgaatggaagcatgccaggtgcatatgagtatttggtatacatgatgtatatatatattaaattattgtcatccaacttagaactatattgttccattcctctcctttattgcatagagaaggatgcggatggtcggggtgttatttataaaataaacggagcaaaaaacggtgggtgatggacgaccacgatttaatattggcaccatacacacgaaaggcttatgtgcatatgagatgttcttatgaagaagagcccaagggttgcttcttatgtccacccaatataatttttacattcatttttatggtcaatataaaagaacacccacaatttataaaaaaatagaaacataaggactatgtgagagcacgtggaaggtctaggtgattccaaaatatcctctaatgtttataaatgtcgtatttctagctttttcctaagtcaactatgcaattttctatcgtgaattcctataaactcgtattgtttaacaacgtccgaattatatattacgaaaatattttttgagtaactcaaacgtaaatcttatataatggtgaattttacaaattccgacaaaattgatagtccgtattggacaaaactaaatcaacaatctattagaaagaaatggagcgagtattagaaagccgtctggaacttatcctcccgcgtgcaagtcgatctttgactttaagcatgagatttgccacttgccagcagcgctccttgtataaataccacgcaaggcgctcgctcgacctccaccattcgaaagatccctccaggaaagatttttcttccctcctccgacgccccagcccaccaacacactctataaagcagccctcagtcacacacagaacgcacaagcgcaagccgggcaagaaaactccgcaggccagtctgcgagttggatggccttcttcttggccctcgtgtgcatcctcatcttgctagccatcgcctcctacgtccagtacactcgctggcaaaaggggaaaggccgcttcggcggccatgggaggtctgctcccttgaagctgcctcctggctccatgggctggccttaccttggcgagaccctccagctttactcccaggaccccagcttcttcttcgcttccaaacagaagaggttagtcgccgtaggcaactactactactcatgcgggcagcgtgttcgtccttcgttctggatccgcccccttgttcacaagctgctaatgattcgaacggaacgaccatgcatgccttgtgtgcaggtacggcgagatcttcaagacgcaccttctgggttgcccgtgcgtgatgctggcgagcccggaggcggcgcggttcgtgctggtgacgcaggcgcacctgttcaagccgacctacccgcggagcaaggagcgcatgatcgggccgtcggctctcttcttccaccagggcgactaccacctccgcctccgcaagctcgtccagggcgcgctcggccccgacgcgctgcgcgcgctcgttcctgaggtggaggccgccgtgcggtccactctcgcttcctgggacgccggccacgtcagaagcacgttccacgccatgaagacggtaaggaataataataatagtcaagcatgcatgcgcggccaattatataatgttggaatgaatcgggtgctgagaattaatacgattgtttgcttctgttgttacgtttcagctgtcgtttgatgtgggcatcgtgacgatcttcggcggccggctggacgagcggcgcaaggcggagctgaggaagaattactccgtcgtggagaaggggtacaactccttccccaacagcctgccggggacgctccattacaaggcgatgcaggtgagcacacacgcgacacggcatttacacaacccatccaacgcattacacgtacggtacgtctcgggcaacggcagtacgtactgccctgcccctggcacgcacgcatgcatgtgacgaaatcgctggacaccgtaccgtacgtacaccgtaggcgcggcggcggctgcacggcgtgctgtgcgacatcatgcgggagcgtcgtggccaggcccaggcggcgggcaccggcctgctgggctgcctgatgcggtcccggggcgacgacggcgcgccgctcctgagcgacgagcagatcgccgacaacgtcatcggcgtgctgttcgcggcgcaggacacgacggccagcgcgctcacctggatcgtcaagtacctccacgaccaccccaagctgctcgaggccgtccgggcggagcaggcggcggtccgcgaggccaccggcggcgggaggcagccgctggcgtgggcgcacacgaagagcatggcgctaacgcatagggtacgagcgtgcgtgctgggaaacgcaaaactggctctttattatttttttcttgtggtttcatccgtacgtcgcccgtccaggtgattttggagagcttaaggatggcgagcatcatctcgttcacgttcagggaggccgtggccgacgtggagtacaaaggtacgcacgcacgtgcgcgcaccacgaagagtagctagaggagcaacgagagtgctttgcttaattctgactcggattatgccgtgtagggttccttatccccaaggggtggaaggtgatgccgctcttcaggaacatccaccacagcccggactacttccaggatccacacaagttcgacccttctagattccaggtacgttacgtacagaagcatgggcctcaccgccgttagttgctgtgggacgacgacgacgtgactgaccggacgttgcgtattatgcaggtggcgccgcgtccgagcacgttcctgccgtttgggcacggcgtgcacgcgtgccccgggaacgagctggccaagctcgagatgctcgtcctcatccaccacctggtcaccggctacaggtgcgtccatctcctctcagatcctctccatatattcccgcttgtcctatagcttgtggaccaggatgacacatggctggctgctgccgctctccatggggctccggctctgatctctctccgtgcatgctccaaatctcctcctgtctgtatgtatgcctgtatcgatcatgtatatactcctgtaccataatctgtggggtcctcgaaatgtacgtcttcactagccccgctgtgctctccctcctatataaactgtggtgatcgactgctataacgacagtttactgatcttacactgagacactgattggcgtctctgcatgctttatttttaaatttgcaggtggcaaatcgttggatccagtgacgaggtcgagtacagcccgttccctgtgcccaagcacggcttgcctgtcagattatggagacaaaacaatccggtcgacagaaaggggcgtgagaccgacgacgatcatgtggagaggatatttatttagtttgactcttgagttaggcatgaatttaaccccaagctagctagagaagttttttttcccctttgaaattcttctttgctcgcctcttcctcctggatcaaattgcgttggaggagaagaaacggcagctttctctctttcgttttctttgcctgcttcaccgctacgataatggtgaaaatatgtaagctacgtggacatcaatgatccacagcatcgttgatatatataatatatagagaaaattctctgcacgatcaatgcaattttatccggtatcttatttaccatagtaaatgtcttaatcctttctatatcactcggatacaatttcttaccttttaatgtagagatgtaaatggaacggatatttttccatccatattcgaattcgatctatctaaaagggctgagattcgattcgtattcaaatccgggtactatctgtatccgtatactcaaaagttggctattcaagatgtcgctattcattcagatcttatccaacacaactagataatatccgtatacgattcgaatccaaagagtaaatataaaaacaaatatggtacaagcaatttccgtccgtaccgattacacccctacttcaatgtcatcgctggtggcaattatatgttcttatattttctatgtcaagattacgaaattatatagtaccccgtttgtttcaagatgtcgctattcattcagatcttatccaacacaactagataatatccgtatacgattcgaatccaaagagtaaatataaaaacaaatacggtacaagcaatttccgcccgtaaccgattacacccctacttcaatgtcatcgttggtggcaattatatgttcttatattttctatgtcaagattatgaaattatatagtaccccgtttgtttcaagatgtcgctattcatttaagggaaatttggatccataccattaaaagatcaccactttggatccataccattactatctcacttacatgtgggtccacatgagtcaatgacatgtggggtccatggtatatatctaaagtttgaatcttttaatggtatagatccaattgttccttcatttaaatcttatccaacacaactagataatatccgtatacgattcgaatccaaagagtaaatatgaaaacaaatacggtacaagcaatttccgcctgtaaccgattacacccctacttcaatgtcatcgttggtggcaattatatgttcttatattttctatgtcaagattacgaaattatatagtaccccgtttgtttcaag

SEQ ID NO: 23 - DNA, 5503 bp, Zea mays
  misc_difference 2719..3218: note = n is a, c, g, or t
  misc_difference 4584..5083: note = n is a, c, g, or t
  source 1..5503: mol_type = unassigned DNA
aagctcctcccctaccgtcgcccagtagactccctattccatttttaatttcaatctctactctataaatagggcaatttacagtataaaacaacattttgcataaccatttacacgacctgttagagatggtctaaggagataaaagatttgcatgagcagaagaagagggaataggagaataaccaagaatatgggtatttggtgtacatgggtttgaagcaagatgaatggagaagggtgaagggaagcatgccaggtgcatatgagtatttgatgtatatattaaattattgtcattcaacttagatctatataacattgttccattcctctcctttattgcatagagaaggatgcggatgatgggggtgttatttataaaataaacggagcgaaaaacggtgggtgttggacgaccacgatttaatattggcaccatatataataatgattatatgcacatgagatgttctcacgaagaagagcccaagggttgcttcttatgtccacccaatataatttttacattcatatttgtggtcaatataaaagaacatccacgatatataaaatatagaaacataaggactatgtgggagcacgtggaagatctaggtgattccaaaatatcctcacatgtttataaaatgtcgtatttctagccttttcctaagtcaactatgttattttctatcgtggattcctataaaatcgtattgtttaacaacatctgaattatatttaacgaaaatattttttgattaactcaaacgtaaatcttataatggtgaattttatagattccgacaaaattgatagtccgtattggacaaaactaaatcaacaatctattagaaagaaatggagggagtattagaaagccgtctggaacttatcctcccgcatgcaagtcgatctttgactttaagcatgagatttgccacttgccagcagcgctccttgtataaataccacgcaaggcgctcgctcgacctccaccattcgaaagatccctccaggaaagatttttcttccctcctccgacgccccagcccaccaacacactctataaagcagcccccagtcacacacagaacgcacaagcgcaagccgggcaagaaaactccgcaggccagtctgcgagttggatggccttcttcttggccctcgtgtgcatcctcgtcttcctagccaccgcctcctacgtccagtacactcgctggcagaaggggaaaggccgcttcggcggccatgggaggtctgctcccttgaagctgcctcctggctccatgggctggccttaccttggcgagaccctccagctttactcccaggaccccagcgtcttcttcgcttccaaacagaagaggttagtcgccgtaggcaactactagtcatgcgggcagcgtgttcgtccttcgttctcgatccgcccccttgttcacaagctgctaatgattcgaacggaacgaccgtgcatgccttgtgtgcaggtacggcgagatcttcaagacgcaccttctgggctgcccgtgcgtgatgctggcgagcccggaggcggcgcggttcgtgctggtgacgcaggcgcacctgttcaagccgacctacccgcggagcaaggagcgcatgatcgggccgtcggctctcttcttccaccagggcgactaccacctccgcctccgcaagctcgtccagggcgcgctcggccccgacgcgctgcgcgcgctcgttcctgaggtggaggccgccgtgcggtccactctcgcttcctgggacgccggccacgtcagaagcacgttccacgccatgaagacggtaaggaataataataatagtcaagcatgcatgcgcggccaattatataatgttggaatgaatcgggtgctgagaattaatacgattgtttgcttctgttgttacgtttcagctgtcgtttgatgtgggcatcgtgacgatcttcggcggccggctggacgagcggcgcaaggcggagctgaggaagaattactccgtcgtggagaaggggtacaactccttccccaacagcctgccggggacgctccattacaaggcgatgcaggtgagcacacacgcgacacggcatttacacaacccatccaacgcattacacgtacggtacgtctcgggcaacggcagtacgtactgccctgcccctggcacgcacgcatgcatgtgacgaaatcgctggacaccgtaccgtacgtacaccgtaggcgcggcggcggctgcacggcgtgctgtgcgacatcatgcgggagcgtcgaggccaggcccaggcgggcaccggcctgctgggctgcctgatgcggtcccggggcgacgacggcgcgccgctgctgagcgacgagcagatcgccgacaacgtcatcggcgtgctgttcgcggcgcaggacacgacggccagcgcgctcacctggatcgtcaagtacctccacgaccaccccaagctgctcgaggccgtccgggcggagcaggcggcggtccgcgaggccaccggcggcgggaggcagccgctggcgtgggcgcacacgaagagcatggcgctaacgcatagggtacgagcgtgcgtgctgggaaacgcaaaactggctcttcattatttttttcttgtggtttcatccgtacgtcgcccgtccaggtgattttggagagcttaaggatggcgagcatcatctcgttcacgttcagggaggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgagcatcatctcgttcacgttcagggaggccgtggccgacgtggagtacaaaggtacgcacgtacgtgcgcgcaccacgaagagtagctagaggagcaacgagagttggtttgcttaattctgactcggattattccatgtatcgatctccttccttggcgtacgtgtagggttccttatccccaaggggtggaaggtgatgcctctcttcaggaacatccaccacagccctgactacttccaggatccacacaagttcgacccttctagattccaggtacgttacgtacgtacagaagcatgggcctcaccgccgttagttgctgtgggacgacgacgacgtgactgaccggacgttgcgtattatgcaggtggcgccgcgtccgagcacgttcctgccgtttgggcacggcgtgcacgcgtgccccgggaacgagctggccaagctcgagatgctcgtcctcatccaccacctggtcaccggctacaggtgcgtccatctcctctcagatcctctccatatattccccgcttgtcctatagcttgtggaccaggatgacacatggctggctgctgccgctctccatggggctccggctctctctctccgtgcatgctccaaatctcctcctgtctgtatgtatgcctgtatcgatcatgtatatactcctgtaccataatctgtggggtcctcgaaatgtacgtcttcactagccccgctgtgctctccttcctcctatatatactatatatatgatgagcatggcgatcgactgctataacgacggtttactgatcttacactgaggcactgattggcgtccctgcatgctttatttttaaatttgcaggtggcaaatcgttggatccagtgacgaggtcgagtacagcccgttccctgtgcccaagcacggcttgcctgtcagattatggagacaaaacaatccggtcgtcgacagaaaggggcgtgagaccgacgacgatgatgtggaggatattttagtttgactcttgagttaggcatgaatttaaccccaagctagctagagaagttttttttccctttgaaattcttctttgctcgccttcctcctggatcaaattacgttggaggacaagaaacggcagctttctctttcgttttctttgcctgcttcaccgcgacgataatggtgaaaatatgtaagctacgtggacatcaatgatccacagcatcgttgatatatataatatagagagaaaattctctgcacggtcaatgcagttttatccggtatcttattgtcggcgtttcgagaccggggtccctgagccgacgagtgaagtgtcgccgcgtgccccaacccagatgggtcgacgcgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncggccttcgtgttcgtcccgcgcccaggtcgggtgcgcttacagtagggggttacaagcgtccgcacgggggaaggagcgagcggcctcacgcgagcgcccgtctcgtcctcgtccccgcgcggccgaccctctctaagagggccctggcccttccttttataggcgtaaggagagggtccaggtgtagaatggggggtgtagcagagtgctacgtgtctagcggaggagggctagcgccctaagtacacgccatcgtggcggccggagagattttggcgcccggcgtgtgatgtcgtggccgtcggaggagcgctggagcctggaggagggacagctgtcggggccgtcgagtccttgctgacgtcctcttgcttccgtaagggggccgagagccgccatcgttagggagcgtgcggga

SEQ ID NO: 24 - DNA, 4617 bp, Zea mays
  source 1..4617: mol_type = unassigned DNA
agtagtaccaaaaccctaaggtacaattgtttgaagacatgccatgtgactcatgttgtccaaatggatatcgtagtttcaatataaatgtgtgacacaatcttttggatggttaagtagttgcatttgtcaactagttcttcagctttacacatgtgttcatgagcagtggcgaagctagaacacagtaggtgcagatgcatagatcaatttttcaccatgtttatgacttaagtctaagcatctatatcgcaagtgcatctctcgtgtttgctctagcttctccatttttcatgagaataaaccgcgttacatgccttcttgggatattctacatcagatccaagtatccaacctacatgataataatgaataaacctccaaacacatttttagcaaactaaatctattactacaggaaaatatggcaacaaccgacttaccttagtaatatttgtgtcgaatgaagcattaacaacaagagattgatcaggttcttaaccatcgatcaggcattgtaccatagatagccattgagcgtctcttgatttaatcatcgggggatagtggcgctctgctggcttattcaatgctatttaacatgaaaggtgtaagaatagtgtatgtatgtggtggggctgagatgtttcctacctcattattacgcttatgattttgcaaaaaatttcataagtcatgttcgttttagtgccaatccatgtggattggagtgggtttaaatccctagcaagtcaaaatcattcttaattattttaaatcccctccaatccatgtgggagaataacccaacaagacctaaagaggcatactaaagacatcccaaatatatatcattcttccaataactttagtagatgaaattgaaattgagttgatcgaccttattttttgttgtttacacatggaacttttgtgacagagttactacactttattaagcaaaaaatatacaagtatttatatggtacatttgaagaggagtttagtcataataatgattcaaaatgtttttctttgtaaggcaacccattgctttttattattggggaaacactgtgatactacatattaattaaaataattttcttgaacatggtgattactttccaggtaccctacgtcaagtctatccatgataattttctaaactggacattctgtaaactcttgtatcctacagaatggatgaagtgaatgaggaaaatgggccacagatacttgttccttctgaatctattgatctagtgacaagtttgtttgattcagaacctcctacatcctttgatttctctaaaagtgtcccggttgttcatagtagacatttaagtgaggatttaagtgcccttacaattaatgatctacgcttgaacaatggagacaacaattgcaatgaccagattgaacagaatggcataaataatcactctagacattttagtgaagatttaagttgccttgcaactaatgatttttatgtaaacaaagaagaggagaatgatcattactcgagtgaacaaaaggtagaaagtctccctaactctgctgaaagaaacatttataaggcagcagagattgctgagaggttcattcagtctatgaataatagggtgtttgtcgacaccggagcaccaatagaatctgtcaaagatgctgtaagcaaatttggaggcattcttgattggaaagaggtaatgtctattttcctaataggttctttatattcctcaataccctttttattttcggtgttaacttagaatattaccatggttttcaattccatttattttttatagagacgtaagcatgtccaaattgaacttgacaaagcactagaagatgctcctaagtaccaaaaaagagcggaagttgcagaagttgagaaaaataaagttgtcatggaattgtgtaacactaggagagccattgaagggttgaagctaaatttggagaagacacaaaatgaggcaatacaagcacaacaagattcagagcttgctgatgtacggttcaaagagattcaacagggaattgccttcagagagagtgccgcagcaagagcagagattgagcttgcaagatatcgctatgctagtgccatggcagaactacatttagtaaaggatgagattgaacaacttcagaaggagtatcaatccttaaacactatgaggtacaatgctgaaacaaaagcatgtgaatctaatgttgcatctcagaagattgaggaaactgtggatgaccttaccctcgaaattattagattaaaagaggagctcacctcttcacaagctactcatattatagcagaggaacaaaaattgaatgttgctttagcaaatcaacaagaaaaggagaaatggcagaatgaactaaggcaagctgatgaagaggtccaaagtttgcgtcatgcaacatcggtcaacaaagatcttgaatctaagctaaaaaatgcctctacacttttggtgaagttgcaagatgagttttctagttatttgaaaggggaatgcacacaagaggttagtatagatggagatgcagagagacaaccattggtgtttatcaagatgaagctagcaaatgctaggaaggagcttgaggacatgagagcagacattaaaaaatccaaggatgatgttaggaaactttggaatgtcgcagctacatttcgggctgatatagatagggaggaggcaggtctactagcattagagcacaaagagcaccttgcttcgatttctgtatcatctcaccaggaagagctaagcaacattacatatgagctcaacatcatccatgaaagaacaaaagcaactaagatgcctatagagctacaacaagcaactgaagttgtggaacaagcaaaagctaaggctttgatggcccattatgaggtggcaaaagctagggaagatgcagatcaagttaagtcacaattaaatgtcattaaattgagactagaagcggcgtcaagggagatacttgcagtgaatgcatctaaagaaattgcaaccacctcagcaaatgcattgcaagaatacaaagatgaagcacatatagagccccaagatgagcagataagaaataactatatgacattatcacttgaagagtatgacgccttgagcaagaaatcccaagatgctgaacgcctcgctaagaagcgagtcatcaaggctattgagaaaatcaaagaagccaaggatgcagaggtgaggagcttgaatcagctagagcagtcgaccaagaagatctatgagaggaagttggagctaagggttgcgcaggagaaagccaattcagcgcaatatgtcaagctaaccatggagaacgagctgagaaagtgcaaggccaagcatgagcaacagaagaaagtgggcgagtcagtccattccatttctgacgtccccaatttgaagagtggatcattgtcttttgacgcagcatcttcaacctccaatcctcacatggttggagcattgtctagagctgacactatagcaacaactagagtgaaagagccaaaaccacgaaagtcactctttccaaggtccgtagtggccatgttcgtgtctaggaagaagacacgttgaccattacattcctatccactaataatattgtgctaaattcttcggtatggttcattcgtaaatgtactcatgtagcacataggttacaattggaacatatgtgtagagttgcttcagaaatatagttcaatttatgttatagattatattgagcaccacacattgagttgaccctcttacctctatgtgaatattgtaagaagcatattgtcacacccagatttaaggacaaatccagatgcatcccatatgtgtgctaggattagatttcacacatacgatgactccatgtatagaaattagtatcacaaatttattaaataatggattgtctgtacaaaaataactaaataaaataagctaaatggatataacttctctccacatgcatagttgaccaggagacgatgacctagaattctttgaactcgtaattataatcatcctctcagggtacatattcctgatatgagcagtaattatagcaagagtgagtacacttacggttggtactcaacaagcatgtaggaaaaactgtagtgtaaggcttaacaaggaaaagtctgaggctaagcattaacttttaattaagttggtcaaacttttattagcaattactaagtataagtaaataccaaccaaataaataaatgatcataaaggagatgacccacaatatataaaatgcatgtacaatttaatttaattccaaaatttactcatgtgaggatctgagccgctcatgaccatgagcatggctaatatatcagttttaccctttgaagaggtcgcacatctttatccataagtcgtgatacccatctgccccaggttagctaggccattcacctcttcctaagaggtagggcagggttcactataaggcctttacaaagttccactaatacgagaaaacccgctacgaattcaagatttggtggaacaagaatccctcgcctaaaaagctatcacagacagaacctccctatactttagcaac

SEQ ID NO: 25 - DNA, 2307 bp, synthetic construct
  misc_feature 1..2307: note = cDNA of ZmWEB1 derived from B73
  source 1..2307: mol_type = other DNA
atggatgaagtgaatgaggaaaatgggccacagatacttgttccttctgaatctattgatctagtgacaagtttgtttgattcagaacctcctacatcctttgatttctctaaaagtgtcccggttgttcatagtagacatttaagtgaggatttaagtgcccttacaattaatgatctacgcttgaacaatggagacaacaattgcaatgaccagattgaacagaatggcataaataatcactctagacattttagtgaagatttaagttgccttgcaactaatgatttttatgtaaacaaagaagaggagaatgatcattactcgagtgaacaaaaggtagaaagtctccctaactctgctgaaagaaacatttataaggcagcagagattgctgagaggttcattcagtctatgaataatagggtgtttgtcgacaccggagcaccaatagaatctgtcaaagatgctgtaagcaaatttggaggcattcttgattggaaagagagacgtaagcatgtccaaattgaacttgacaaagcactagaagatgctcctaagtaccaaaaaagagcggaagttgcagaagttgagaaaaataaagttgtcatggaattgtgtaacactaggagagccattgaagggttgaagctaaatttggagaagacacaaaatgaggcaatacaagcacaacaagattcagagcttgctgatgtacggttcaaagagattcaacagggaattgccttcagagagagtgccgcagcaagagcagagattgagcttgcaagatatcgctatgctagtgccatggcagaactacatttagtaaaggatgagattgaacaacttcagaaggagtatcaatccttaaacactatgaggtacaatgctgaaacaaaagcatgtgaatctaatgttgcatctcagaagattgaggaaactgtggatgaccttaccctcgaaattattagattaaaagaggagctcacctcttcacaagctactcatattatagcagaggaacaaaaattgaatgttgctttagcaaatcaacaagaaaaggagaaatggcagaatgaactaaggcaagctgatgaagaggtccaaagtttgcgtcatgcaacatcggtcaacaaagatcttgaatctaagctaaaaaatgcctctacacttttggtgaagttgcaagatgagttttctagttatttgaaaggggaatgcacacaagaggttagtatagatggagatgcagagagacaaccattggtgtttatcaagatgaagctagcaaatgctaggaaggagcttgaggacatgagagcagacattaaaaaatccaaggatgatgttaggaaactttggaatgtcgcagctacatttcgggctgatatagatagggaggaggcaggtctactagcattagagcacaaagagcaccttgcttcgatttctgtatcatctcaccaggaagagctaagcaacattacatatgagctcaacatcatccatgaaagaacaaaagcaactaagatgcctatagagctacaacaagcaactgaagttgtggaacaagcaaaagctaaggctttgatggcccattatgaggtggcaaaagctagggaagatgcagatcaagttaagtcacaattaaatgtcattaaattgagactagaagcggcgtcaagggagatacttgcagtgaatgcatctaaagaaattgcaaccacctcagcaaatgcattgcaagaatacaaagatgaagcacatatagagccccaagatgagcagataagaaataactatatgacattatcacttgaagagtatgacgccttgagcaagaaatcccaagatgctgaacgcctcgctaagaagcgagtcatcaaggctattgagaaaatcaaagaagccaaggatgcagaggtgaggagcttgaatcagctagagcagtcgaccaagaagatctatgagaggaagttggagctaagggttgcgcaggagaaagccaattcagcgcaatatgtcaagctaaccatggagaacgagctgagaaagtgcaaggccaagcatgagcaacagaagaaagtgggcgagtcagtccattccatttctgacgtccccaatttgaagagtggatcattgtcttttgacgcagcatcttcaacctccaatcctcacatggttggagcattgtctagagctgacactatagcaacaactagagtgaaagagccaaaaccacgaaagtcactctttccaaggtccgtagtggccatgttcgtgtctaggaagaagacacgttga

SEQ ID NO: 26 - protein, 768 aa, Zea mays
MDEVNEENGPQILVPSESIDLVTSLFDSEPPTSFDFSKSVPVVHSRHLSEDLSALTINDLRLNNGDNNCNDQIEQNGINNHSRHFSEDLSCLATNDFYVNKEEENDHYSSEQKVESLPNSAERNIYKAAEIAERFIQSMNNRVFVDTGAPIESVKDAVSKFGGILDWKERRKHVQIELDKALEDAPKYQKRAEVAEVEKNKVVMELCNTRRAIEGLKLNLEKTQNEAIQAQQDSELADVRFKEIQQGIAFRESAAARAEIELARYRYASAMAELHLVKDEIEQLQKEYQSLNTMRYNAETKACESNVASQKIEETVDDLTLEIIRLKEELTSSQATHIIAEEQKLNVALANQQEKEKWQNELRQADEEVQSLRHATSVNKDLESKLKNASTLLVKLQDEFSSYLKGECTQEVSIDGDAERQPLVFIKMKLANARKELEDMRADIKKSKDDVRKLWNVAATFRADIDREEAGLLALEHKEHLASISVSSHQEELSNITYELNIIHERTKATKMPIELQQATEVVEQAKAKALMAHYEVAKAREDADQVKSQLNVIKLRLEAASREILAVNASKEIATTSANALQEYKDEAHIEPQDEQIRNNYMTLSLEEYDALSKKSQDAERLAKKRVIKAIEKIKEAKDAEVRSLNQLEQSTKKIYERKLELRVAQEKANSAQYVKLTMENELRKCKAKHEQQKKVGESVHSISDVPNLKSGSLSFDAASSTSNPHMVGALSRADTIATTRVKEPKPRKSLFPRSVVAMFVSRKKTR

SEQ ID NO: 27 - DNA, 2613 bp, Zea mays
  source 1..2613: mol_type = unassigned DNA
agtttagtcataataattattcaaaatgtttttctttgtaaggcaacccattgctttttattattggggaaacactgtgatactacgtattaattaaaataattttcttgaacatggtgattactttccaggtaccctacgtcaagtctatccatgataattttctaaactggacattctgtaaactcttgtatcctacagaatggatgaagtgaatgaggaaaatgggccgcagatacttgttccttctgaatctattgatccagtgacaagtttgtttgattcagaacctcctacatcctttgatttctctaaaagtgtcccggttgttcatagtagacatttaagtgaggatttaagtgcccttacaattaatgatctacgcttgaacaatggagacaacaattgcaatgaccagattgaacagaatggcataaataatcactctagacattttagtgaagatttaagttgccttgcaactaatgatttttatgtaaacaaagaagaggagaatgatcattactcgagtgaacaaaaggtagaaagtctccctaactctgctgaaagaaacatttataaggcagcagagattgctgagaggttcattcagtctatgaataatagggtgtttgtcgacaccggagcaccaatagaatctgtcaaagatgctgtaagcaaatttggaggcattcttgattggaaagaggtaatgtctattttcctaataggttctttatattcctcaatacccttttttcggtgttaacttagaatattaccatggttttcaattccatttattttttatagagacgtaagcatgtccaaattgaacttgacaaagcactagaagatgctcctaagtaccaaaaaagagcggaagttgcagaagttgagaaaaataaagttgtcatggaattgtgtaacactaggagagccattgaagggttgaagctaaatttggagaagacacaaaatgaggcaatacaagcacaacaagattcagagcttgctgatgtacggttcaaagagattcaacagggaattgccttcagagagagtgccgcagcaagagcagagattgagcttgcaagatatcgctatgctagtgccatggcagaactacatttagtaaaggatgagattgaacaacttcagaaggaatatcaatccttaaacactatgaggtacaatgctgaaacaaaagcatgtgaatctaatgttgcatctcagaagattgaggaaactgtggatgaccttaccctcgaaattattagattaaaagaggagctcacctcttcacaagctactcatattatagcagaggaacaaaaattgaatgttgctttagcaaatcaacaagaaaaggagaaatggcagaatgaactaaggcaagctgatgaagaggtccaaagtttgcgtcatgcaacatcggtcaacaaagatcttgaatctaagctaaaaaatgcctctacacttttggtgaagttgcaagatgagttttctagttatttgaaaggggaatgcacacaagaggttagtatagatggagatgcagagagacaaccattggtgtttatcaagatgaagctagcaaatgccaggaaggagcttgaggacatgagagcagacattaaaaaatccaaggatgatgttaggaaactttggaatgtcgcagctacatttcgggctgatatagatagggaggaggcaggtctactagcattagagcacaaagagcaccttgcttcgatttctgtatcatctcaccaggaagagctaagcaacattacatatgagctcaacatcatccatgaaagaacaaaagcaactaagatgcctatagagctacaacaagcaactgaagttgtggaacaagcaaaagctaaggctttgatggcccattatgaggtggcaaaagctagggaagatgcggatcaagttaagtcacaattaaatgtcattaaattgagactagaagcggcatcaagggagatacttgcagtgaatgcatctaaagaaattgcaaccacctcagcaaatgcattgcaagaatacaaagatgaagcacatatagagccccaagatgagcagataagaaataactatatgacattatcacttgaagattatgatgccttgagcaagaaatcccaagatgctgaacgcctcgctaagaagcgagtcatcaaggctattgagaaaatcaaagaagccaaggatgcagaggtgaggagcttgaatcagctagagcagtcaaccaagaagatctatgagaggaagttggagctaagggttgcgcaggagaaagccaattcagcacaatatgtcaagctaaccatggagaacgagctgagaaagtgcaaggccaagcatgagcaacagaagaaagtgggcgagtcagtccattccatttctgacgtccccaatttgaagagtggatcattgtcttttgacgcagcatcttcaacctccaatcctcacatggttggagcattgtctagagctgacactatagcaacaactagagtgaaagagccaaaaccacgaaagtcactctttccaaggtccgtagtggccatgttcgtgtctaggaagaagacacgttga

SEQ ID NO: 28 - DNA, 2304 bp, synthetic construct
  misc_feature 1..2304: note = cDNA of ZmWEB1 derived from PH207
  source 1..2304: mol_type = other DNA
atggatgaagtgaatgaggaaaatgggccgcagatacttgttccttctgaatctattgatccagtgacaagtttgtttgattcagaacctcctacatcctttgatttctctaaaagtgtcccggttgttcatagtagacatttaagtgaggatttaagtgcccttacaattaatgatctacgcttgaacaatggagacaacaattgcaatgaccagattgaacagaatggcataaataatcactctagacattttagtgaagatttaagttgccttgcaactaatgatttttatgtaaacaaagaagaggagaatgatcattactcgagtgaacaaaaggtagaaagtctccctaactctgctgaaagaaacatttataaggcagcagagattgctgagaggttcattcagtctatgaataatagggtgtttgtcgacaccggagcaccaatagaatctgtcaaagatgctgtaagcaaatttggaggcattcttgattggaaagagagacgtaagcatgtccaaattgaacttgacaaagcactagaagatgctcctaagtaccaaaaaagagcggaagttgcagaagttgagaaaaataaagttgtcatggaattgtgtaacactaggagagccattgaagggttgaagctaaatttggagaagacacaaaatgaggcaatacaagcacaacaagattcagagcttgctgatgtacggttcaaagagattcaacagggaattgccttcagagagagtgccgcagcaagagcagagattgagcttgcaagatatcgctatgctagtgccatggcagaactacatttagtaaaggatgagattgaacaacttcagaaggaatatcaatccttaaacactatgaggtacaatgctgaaacaaaagcatgtgaatctaatgttgcatctcagaagattgaggaaactgtggatgaccttaccctcgaaattattagattaaaagaggagctcacctcttcacaagctactcatattatagcagaggaacaaaaattgaatgttgctttagcaaatcaacaagaaaaggagaaatggcagaatgaactaaggcaagctgatgaagaggtccaaagtttgcgtcatgcaacatcggtcaacaaagatcttgaatctaagctaaaaaatgcctctacacttttggtgaagttgcaagatgagttttctagttatttgaaaggggaatgcacacaagaggttagtatagatggagatgcagagagacaaccattggtgtttatcaagatgaagctagcaaatgccaggaaggagcttgaggacatgagagcagacattaaaaaatccaaggatgatgttaggaaactttggaatgtcgcagctacatttcgggctgatatagatagggaggaggcaggtctactagcattagagcacaaagagcaccttgcttcgatttctgtatcatctcaccaggaagagctaagcaacattacatatgagctcaacatcatccatgaaagaacaaaagcaactaagatgcctatagagctacaacaagcaactgaagttgtggaacaagcaaaagctaaggctttgatggcccattatgaggtggcaaaagctagggaagatgcggatcaagttaagtcacaattaaatgtcattaaattgagactagaagcggcatcaagggagatacttgcagtgaatgcatctaaagaaattgcaaccacctcagcaaatgcattgcaagaatacaaagatgaagcacatatagagccccaagatgagcagataagaaataactatatgacattatcacttgaagattatgatgccttgagcaagaaatcccaagatgctgaacgcctcgctaagaagcgagtcatcaaggctattgagaaaatcaaagaagccaaggatgcagaggtgaggagcttgaatcagctagagcagtcaaccaagaagatctatgagaggaagttggagctaagggttgcgcaggagaaagccaattcagcacaatatgtcaagctaaccatggagaacgagctgagaaagtgcaaggccaagcatgagcaacagaagaaagtgggcgagtcagtccattccatttctgacgtccccaatttgaagagtggatcattgtcttttgacgcagcatcttcaacctccaatcctcacatggttggagcattgtctagagctgacactatagcaacaactagagtgaaagagccaaaaccacgaaagtcactctttccaaggtccgtagtggccatgttcgtgtctaggaagaagacacgt

SEQ ID NO: 29 - protein, 768 aa, Zea mays
MDEVNEENGPQILVPSESIDPVTSLFDSEPPTSFDFSKSVPVVHSRHLSEDLSALTINDLRLNNGDNNCNDQIEQNGINNHSRHFSEDLSCLATNDFYVNKEEENDHYSSEQKVESLPNSAERNIYKAAEIAERFIQSMNNRVFVDTGAPIESVKDAVSKFGGILDWKERRKHVQIELDKALEDAPKYQKRAEVAEVEKNKVVMELCNTRRAIEGLKLNLEKTQNEAIQAQQDSELADVRFKEIQQGIAFRESAAARAEIELARYRYASAMAELHLVKDEIEQLQKEYQSLNTMRYNAETKACESNVASQKIEETVDDLTLEIIRLKEELTSSQATHIIAEEQKLNVALANQQEKEKWQNELRQADEEVQSLRHATSVNKDLESKLKNASTLLVKLQDEFSSYLKGECTQEVSIDGDAERQPLVFIKMKLANARKELEDMRADIKKSKDDVRKLWNVAATFRADIDREEAGLLALEHKEHLASISVSSHQEELSNITYELNIIHERTKATKMPIELQQATEVVEQAKAKALMAHYEVAKAREDADQVKSQLNVIKLRLEAASREILAVNASKEIATTSANALQEYKDEAHIEPQDEQIRNNYMTLSLEDYDALSKKSQDAERLAKKRVIKAIEKIKEAKDAEVRSLNQLEQSTKKIYERKLELRVAQEKANSAQYVKLTMENELRKCKAKHEQQKKVGESVHSISDVPNLKSGSLSFDAASSTSNPHMVGALSRADTIATTRVKEPKPRKSLFPRSVVAMFVSRKKTR

SEQ ID NO: 30 - DNA, 4617 bp, Zea mays; identical to SEQ ID NO: 24
  source 1..4617: mol_type = unassigned DNA

SEQ ID NO: 31 - DNA, 4613 bp, Zea mays
  source 1..4613: mol_type = unassigned DNA
agtagtaccaaaaccctatggtacaattgtttgaagacatgccatgtgactcatgttgtccaaatggatatcgtagtttcaatataaatgtgtgacacaatcttttggatggttaagtagttgcatctgtcaactagttcttcagctttacacatgtgttcatgagcagtggcgaagctagaacacagtaggtgcagatgcatagatcaatttttcaccatgtttatgacttaagtctaagcatctatatcgcaagtgcatctctcgtgtttgctctagcttctccatttttcatgagaataaactgcgttacatgccttcttgggatattctacatcagatccaagtatccaacctatatgataataatgaataaacctccaaacacatttttagcaaactaaatctattactacaggaaaatatggcaacaaccgacttaccttagtaatatttgtgtcgaatgaagcattaacaacaagagattgatcaggttcttaaccatcgatcaggcattgtaccatagatagccattgagcgtctcttgatttaatcgtcgggggatagtggcgctctgctggcttattcaatgctatttaacatgaaaggtgtaagaatagtgtatgtatgtggtggggctgagatgtttcctacctcattattacgcttatgattttgcaaaaaatttcataagtcatgttcgttttagtgccaatccatgtggattggagtgggtttaaatccctagcaagtcaaaatcattcttaattattttaaatcccctccaatccatgtgggagaataacccaacaagacctaaagaggcatactaaagacatcccaaatatatatcattcttccaataactttagtagatgaaattgaaattgagttgatcgaccttattttttgttgtttacacatggaacttttgtgacagagttactacactttattaagcaaaaaatatacaagtatttatatggtacatttgaagaggagtttagtcataataattattcaaaatgtttttctttgtaaggcaacccattgctttttattattggggaaacactgtgatactacgtattaattaaaataattttcttgaacatggtgattactttccaggtaccctacgtcaagtctatccatgataattttctaaactggacattctgtaaactcttgtatcctacagaatggatgaagtgaatgaggaaaatgggccgcagatacttgttccttctgaatctattgatccagtgacaagtttgtttgattcagaacctcctacatcctttgatttctctaaaagtgtcccggttgttcatagtagacatttaagtgaggatttaagtgcccttacaattaatgatctacgcttgaacaatggagacaacaattgcaatgaccagattgaacagaatggcataaataatcactctagacattttagtgaagatttaagttgccttgcaactaatgatttttatgtaaacaaagaagaggagaatgatcattactcgagtgaacaaaaggtagaaagtctccctaactctgctgaaagaaacatttataaggcagcagagattgctgagaggttcattcagtctatgaataatagggtgtttgtcgacaccggagcaccaatagaatctgtcaaagatgctgtaagcaaatttggaggcattcttgattggaaagaggtaatgtctattttcctaataggttctttatattcctcaatacccttttttcggtgttaacttagaatattaccatggttttcaattccatttattttttatagagacgtaagcatgtccaaattgaacttgacaaagcactagaagatgctcctaagtaccaaaaaagagcggaagttgcagaagttgagaaaaataaagttgtcatggaattgtgtaacactaggagagccattgaagggttgaagctaaatttggagaagacacaaaatgaggcaatacaagcacaacaagattcagagcttgctgatgtacggttcaaagagattcaacagggaattgccttcagagagagtgccgcagcaagagcagagattgagcttgcaagatatcgctatgctagtgccatggcagaactacatttagtaaaggatgagattgaacaacttcagaaggaatatcaatccttaaacactatgaggtacaatgctgaaacaaaagcatgtgaatctaatgttgcatctcagaagattgaggaaactgtggatgaccttaccctcgaaattattagattaaaagaggagctcacctcttcacaagctactcatattatagcagaggaacaaaaattgaatgttgctttagcaaatcaacaagaaaaggagaaatggcagaatgaactaaggcaagctgatgaagaggtccaaagtttgcgtcatgcaacatcggtcaacaaagatcttgaatctaagctaaaaaatgcctctacacttttggtgaagttgcaagatgagttttctagttatttgaaaggggaatgcacacaagaggttagtatagatggagatgcagagagacaaccattggtgtttatcaagatgaagctagcaaatgccaggaaggagcttgaggacatgagagcagacattaaaaaatccaaggatgatgttaggaaactttggaatgtcgcagctacatttcgggctgatatagatagggaggaggcaggtctactagcattagagcacaaagagcaccttgcttcgatttctgtatcatctcaccaggaagagctaagcaacattacatatgagctcaacatcatccatgaaagaacaaaagcaactaagatgcctatagagctacaacaagcaactgaagttgtggaacaagcaaaagctaaggctttgatggcccattatgaggtggcaaaagctagggaagatgcggatcaagttaagtcacaattaaatgtcattaaattgagactagaagcggcatcaagggagatacttgcagtgaatgcatctaaagaaattgcaaccacctcagcaaatgcattgcaagaatacaaagatgaagcacatatagagccccaagatgagcagataagaaataactatatgacattatcacttgaagattatgatgccttgagcaagaaatcccaagatgctgaacgcctcgctaagaagcgagtcatcaaggctattgagaaaatcaaagaagccaaggatgcagaggtgaggagcttgaatcagctagagcagtcaaccaagaagatctatgagaggaagttggagctaagggttgcgcaggagaaagccaattcagcacaatatgtcaagctaaccatggagaacgagctgagaaagtgcaaggccaagcatgagcaacagaagaaagtgggcgagtcagtccattccatttctgacgtccccaatttgaagagtggatcattgtcttttgacgcagcatcttcaacctccaatcctcacatggttggagcattgtctagagctgacactatagcaacaactagagtgaaagagccaaaaccacgaaagtcactctttccaaggtccgtagtggccatgttcgtgtctaggaagaagacacgttgaccattacattcctatccactaataatattgtgctaaattcttcggtatggttcattcgtaaatgtactcatgcagcacataggttacaattggaacatatgtgtagagttgcttcagaaatatagttcaatttatgttatagattatattgagcaccacacattgagttgaccctcttacctctatgtgaatattgtaagaagcatattgtcacacccagatttaaggacaaatccagatgcatcccatatgtgtgctaggattagatttcacacatacgatgactccatgtatagaaatcagtatcacaaatttattaaataatgaattgtctgtacaaaaataactaaataaaataagctaaatggatataacttctctccacatgcatagttgaccaagagacgatgacctagaattctttgaactcgtaattataatcatcctctcatggtacatgttcctgatatgagcagtaattatagcaagagtgagtacacttacggttggtactcaacaagcatgtaggaaaaactgtagtgtaaggcttagcaaggaaaagtctgaggctaagcattaacttttaattaagttggtcaaacttttattagcaattacgaagtataagtaaataccaaccaaataaataaatgatcataaaggagatgacccacaatatataaaatgcatgtacaatttaatttaattccaaaatttactcatgtgaggatctgagccgctcatgaccatgagcatggctaatatatcagttttaccctttgaagaggtcgcacatctttatccataagtcgtgatacccatctgccccaggttagctaggccattcacctcttcctaagaggtagggcaggattcactataaggcctttacaaagttccactagtatgagaaaacccgctacgaattcaagatttggtggaacaagaatcccccgcctaaaaagctatcacagacagaacctccctatactttagcaac

SEQ ID NO: 32 - DNA, 726 bp, Zea mays
  source 1..726: mol_type = unassigned DNA
ctctttcctctcgctccctctgcttctggatacttccgatgcctctcctcgctccctcggtcgctgctgatgcgccacgccgccaccggcgcctcatcggcaggaatcatatggacagcccagaggtggtggcctccgcgtccccggctgcggcggcgagcgagcgccaggagaggcccgcgaggctgcggcggaagatctcgtccgcgttctgcgcctgcatgggccatcccccggcgtcgcacgtgcagcagtaggcatgctacggtgcgtactcaaatctaccagctgcgtgcggtccgtcgtggggtatggctgacgagcggcctggggttctcgctctctttcttgctatgcaacaaacatttgtgatttgtgcttacacgtgagaccgtgcttgctgtaagatttttctcccctcttttctgtggaggtcataggaatccctccacatcgccctctccccgttgtccctctcccgtcacttcatcttctcttccagtcgtttcatctttctcggcagctttggcttcagtcatcggcttcgcaagttcgcatcgacatggggttctaattggacaacctaaggcttacaacgagccgtggtctaaccacccaaactgactgctcagtgacagaaatcagacaggttggtatctaatgtttaaatataatggcataaattctagcaccttgaaaaagacaaactcagcacgccccattgtg

SEQ ID NO: 33 - DNA, 303 bp, synthetic construct
  misc_feature 1..303: note = cDNA of GRMZM2G397260 derived from B73
  source 1..303: mol_type = other DNA
atgcgccacgccgccaccggcgcctcatcggcaggaatcatatggacagcccagaggtggtggcctccgcgtccccggctgcggcggcgagcgagcgccaggagaggcccgcgaggctgcggcggaagatctcgtccgcgttctgcgcctgcatgggccatcccccggcgtcgcacgtgcagcagtaggcatgctacggtgcgtactcaaatctaccagctgcgtgcggtccgtcgtggggtatggctgacgagcggcctggggttctcgctctctttcttgctatgcaacaaacatttgtga

SEQ ID NO: 34 - protein, 100 aa, Zea mays
MRHAATGASSAGIIWTAQRWWPPRPRLRRRASARRGPRGCGGRSRPRSAPAWAIPRRRTCSSRHATVRTQIYQLRAVRRGVWLTSGLGFSLSFLLCNKHL

SEQ ID NO: 35 - DNA, 2726 bp, Zea mays
  source 1..2726: mol_type = unassigned DNA
agacccaaaatacattccgaaattgaagctgggggtgagaaaatcgtgtgctgctaccctggagagattgagagcaatactcccacgctttttagcatatttttcgtcttcattagatcccccatttttttaaactagaattgcctaggcgtagtgatcctccaaggcaatgctcaccaccgacgttctcctggcaatgctgaatcgcttcttcaggaacgtcgatgatgtgctcgcgcacgccgccggacgaaccatcctagacctcacgtagctggctacttttgcactgcgatcatgcggtgtccaactgctaatttgtacagcttgcttctttacaaagttcctggctgcagactgctctgcaattgattatatcgatggttgatctgaactctcaagcatccgagtaacttgttcagtataggtacgactttgttttggattaaagtcagctgttccagatacaatccatagagataaaacactgtataaatagtagaagacgacacgaattaaaaccaggcgatgttgctacaaagtactgtctgacagtattggttatttggccatagcagaaacatgaatgaatcaatcagaactggtttacagagcaaatacagagctgtgtttgccaaacgaacatgcagatgtcttcgcgaggtagaatgcctattactggcagtcagtgtacaggcaatttcataaacaccacacgctttgaactgtccacgtatatgctgctactatatatttgcttaaaagttccacaactatgagactaaaaacagaagcagaatctgtctgcccataacaaaaacttggactatgcaatgtacgtttctgcatcatgcctgtagaaactcgttatttcttttcttatatattaaagccggacagaaacgtgaacgatgaggccatccacgtcgtcaaaaaaaatcgggccgtccgatcgccaatcaaaggttgatgtctcctcttgctctctcgctcgctccgcctctttcctctcgctccctctgcttctggatacttccgatgcctctcctcgctccctcggtcgctgctgatgcgccacgccgccaccggcgcctcatcggcaggaatcatatggacagcccagaggtggtggcctccgcgtccccggctgcggcggcgagcgagcgccaggagaggcccgcgaggctgcggcggaagatctcgtccgcgttctgcgcctgcatgggccatcccccggcgtcgcacgtgcagcagtaggcatgctacggtgcgtactcaaatctaccagctgcgtgcggtccgtcgtggggtatggctgacgagcggcctggggttctcgctctctttcttgctatgcaacaaacatttgtgatttgtgcttacacgtgagaccgtgcttgctgtaagatttttctcccctcttttctgtggaggtcataggaatccctccacatcgccctctccccgttgtccctctcccgtcacttcatcttctcttccagtcgtttcatctttctcggcagctttggcttcagtcatcggcttcgcaagttcgcatcgacatggggttctaattggacaacctaaggcttacaacgagccgtggtctaaccacccaaactgactgctcagtgacagaaatcagacaggttggtatctaatgtttaaatataatggcataaattctagcaccttgaaaaagacaaactcagcacgccccattgtgcagctgatcattttttagcttatggaaaattcataatattaaacgctggagcagagcttgcctcacatgctagggtcttagagcatctccaagagcaagcaatgtattcaatattgattaattttttcaatatttcaaatgtttatcgatattattgagctgtaaatttatgaattcataaacgagattttctgtctgctaaatggttgcttgccaaagatttgtgattagaagcaaacaaacttaatcttcaatagcaattctgttccacacatggccctgatccatgtctcggttttactttcaaccggaggatttggaattctggaaccaaggaatcagatacagagaaatagaagaacatatagaatagattagtttagggcagatttagagtttgttattttagcccaatggtggatgttgtctcttctaaacattcgcactactactttctatatattaagaatgggagccaatctggaatgctagaccatccgcatcatttttatgtcgttcgatcatccattaactatctgattgtatcagtcacatatgagtttaactgtatgattgccttttaatatcgtaggattattgtgttcattggatttgactgtctgattgtgtcagtcaaatttgagttcattttgtttgcgagctagaatgaaaaacaaaattttgagtggctcaaacaggacttacactggttgcctgcatatatatcgcgggattctcacgtcgttgtttcatatttcattcgcacgaatggttctcgatggccaggacacaacctctgaattttaggccacaaccaactgcggtcgaaggagcaagtatttttacatacttctgcccgactccaaccttgctggatgccgcagtgcgcggctcagtcggtgctcactattgatatgaaaacaaccactgtgacatgttttgggacaccatccctattgcaaataataaaacatacttatcttcaaacatttagtaacta

SEQ ID NO: 36 - DNA, 3702 bp, Zea mays
  source 1..3702: mol_type = unassigned DNA
aggcgatgagcgcctctcgcgacgccagggcggagagctagccggccgggagcccacacgcagctggaagcaccagaccgatcgtgccggccgagcggcggcgcaggcgcaggcgcttacatgggagtagaggcgggcgggtgcgggcggagggcggtcgtcaccgggttctacgtctggggctgggagttcctcaccgccctccttctcttctcggccgccgtcgccgccgcagactcctactagcaagctaccaaccttctttctttcattcccttaggtagctcagccgtacacacaacaacacacaagtcatcagttactagctagttagtagcctatacaacacatacatacatacaaaggtgagtgaggttcgcgtgcaagcagagccaatcgtgccgatcgagctatatacatagccggcggcgagggatgggagaagcggccgcggccgtggcggcgtcgaagaggggcggcgggccggcgccgttcctgaccaagacgcaccagatggtggaggagcggggcacggacgaggtgatctcgtgggcggagcagggccgctccttcgtggtgtggaagcccgtggagctggcgcgcgacctcctcccgctccacttcaagcactgcaacttctcctccttcgtccgccagctcaacacctacgtgagtacactacgccgccgctccggccatcatctcttctactacgatcgatgcaatatatcacctgtcgtcgtcgtttagtgattgcaaaacacatacacttggtttccgtattaaattaatcagctagctagctagatgatcgttctctgctctatgatctgttagttctgaagcatgttgttgttttcgtctgtgctcgataaattaagctatgttatgtggtcgacgagcgagccttccaggcagctaccgtaccgtcttccaaggagtatatgcgtgtgagcgtgtcacggttcgtaggaaggagtgcgtcagtcatgacacatctctaccaccctttaattcctttcccacgcaaagcatgcttgtcgtttcagagctagctgaagaggaatgacctgcgataacacttgaagattagggtgccggtgcgggtctgaaattacacctgtgggtacgatcgtgatttagatagacgacttcacggatgtgattacaggagtttttttttttctctacctgatctaaggccctgtttgggaacacagttttttcaaactgcagtttttcaaatactaaagtatactttagtcatgacattactacagtttacaatgcttcagttttcgaatacaacagtattcaatacatcaaggtgtttgggaaaaactttggttgagaccaatcagccagagcgggaccaagctggcactctctttacagagaaaaactttggctgagaccaaagtttccaaaactgcaaaacaagtgcagtatttgcaatactacagtttagtatacagagatttcagatgagtttccaaacacctcaaagtatataataccacagtattgctcaatactacagtattgcttcaatactgcagaaaaactttgttcccaaacaccccctaaactgccatctctaactactatatatgtatagagcaaggtgcacggggaaattgaataagcaaagcaaatcaggtcggttgacacgccacggtattgtagtggcgacagaagcatggtattctatggaacagttaaggccctgtttgggaacaaagtttttgaaaaccacagtttttgaaatactatactatactttagttatgacaataccgtagtttataataccgcagttttgaaaactgaggtccagagctaagtttagaatgccttaaaacaactatagtatttgcaatacttcagttttgaaaacagagattttacctagcttgccaaacaccattatgtatataatactgcagtatttgagaatactgcagtattcttccaaaactgcagaaaaactttgttcccaaacaccccctaagaagcttcggatggacgagcttttcagggctagctcttctgcgtggcctacaagaaggttaatttagctaggaattggatgctattagctgagcaagcaatataatcatccaaggcatccagcaagtatactaatcttttgttgcctcttccatctattagctgggatacgaaatcgctcaagaaattgacttggaagttaggatgatgatttaggccctgtttgtagtttctccaacagctagcttcataatttgtttttgttttttggctggatagtattttccaaaatagcttcatggtatttggtaaagcttcttctttttttctctctctcaagccaaaggaaagtgatgcagggatacgaatagctgaaacacgagtagcttattctagcgcagtcaaagattcacactgacttgggttcgttctcactgaaccttaatctattaatcagagggagagagagctagcttctctaaatcaatgtgtgaacagctataaggcgttatctgaccatgtgagcgacgtatggtggtcaaagtagacaggcctgacgtgttcatttcggcgtttgtttagggactggctgaataggacactgtgtcgaatgcagctcttgttctttttgccgcattggatacttacgtcgacggcgaccatggcgcatgcgcatccatatccatgcagggtttccgaaaggtggtgccggaccggtgggagttcgcgaacgacaacttccgtcgaggcgagcagggtctcctgtccggcatccgccgccgcaagtcaacggcgctgcagatgtccaagtccggatccggcggcagcggcggcgtgaacgccacgttccccccgcctctgccccctccgcctcccgcgtcggccaccacgtccggcgtccacgagcgcagctcgtcgtcggcgtcgtcgccaccgcgggcgcccgacctggccagcgagaacgagcagctcaagaaggacaaccacacgctgtccgccgagctggcgcaggcgcgccggcactgcgaggagctcctgggcttcctctcgcgcttcctcgacgtccggcagctcgacctccggctgctcatgcaggaggacgtgcgagcgggggcaagcgacgacggcgcacagcgccgcgcgcacgcagtggccagccagctggagcgcggcggcggcgaggaggggaagagcgtgaagctgttcggcgtactcttaaaggacgccgcgaggaagaggggccggtgcgaggaagcggcggccagcgagcggcccatcaagatgatcagggtcggcgagccgtgggtcggcgtcccgtcgtcgggcccgggccggtgcggcggcgagaattaactgtcatccaatgtgaggttgatgacaaggacagtttcatccatcatatcgagcaagtaacaaagccagtgctgtggtaaaactgcaaagacagaacacaggacacaggagaaatataggcgtaagcatgttaattaagaattaattatatatgggatgcttttgaagtagcaagattggaagtagagataagtaaaacgggctagaagcagcgcccatgtgttcagaatggaaaattagcgtttccgtgtgtgtgttaagaaaaacttatatgcgctttctgcgagcacggttgattcttaagagcgacagcaaatgaaaggtgtattattaattgaa

SEQ ID NO: 37 - DNA, 897 bp, synthetic construct
  misc_feature 1..897: note = cDNA of ZmHsftf21 derived from B73
  source 1..897: mol_type = other DNA
atgggagaagcggccgcggccgtggcggcgtcgaagaggggcggcgggccggcgccgttcctgaccaagacgcaccagatggtggaggagcggggcacggacgaggtgatctcgtgggcggagcagggccgctccttcgtggtgtggaagcccgtggagctggcgcgcgacctcctcccgctccacttcaagcactgcaacttctcctccttcgtccgccagctcaacacctacggtttccgaaaggtggtgccggaccggtgggagttcgcgaacgacaacttccgtcgaggcgagcagggtctcctgtccggcatccgccgccgcaagtcaacggcgctgcagatgtccaagtccggatccggcggcagcggcggcgtgaacgccacgttccccccgcctctgccccctccgcctcccgcgtcggccaccacgtccggcgtccacgagcgcagctcgtcgtcggcgtcgtcgccaccgcgggcgcccgacctggccagcgagaacgagcagctcaagaaggacaaccacacgctgtccgccgagctggcgcaggcgcgccggcactgcgaggagctcctgggcttcctctcgcgcttcctcgacgtccggcagctcgacctccggctgctcatgcaggaggacgtgcgagcgggggcaagcgacgacggcgcacagcgccgcgcgcacgcagtggccagccagctggagcgcggcggcggcgaggaggggaagagcgtgaagctgttcggcgtactcttaaaggacgccgcgaggaagaggggccggtgcgaggaagcggcggccagcgagcggcccatcaagatgatcagggtcggcgagccgtgggtcggcgtcccgtcgtcgggcccgggccggtgcggcggcgagaattaa

SEQ ID NO: 38 - protein, 298 aa, Zea mays
MGEAAAAVAASKRGGGPAPFLTKTHQMVEERGTDEVISWAEQGRSFVVWKPVELARDLLPLHFKHCNFSSFVRQLNTYGFRKVVPDRWEFANDNFRRGEQGLLSGIRRRKSTALQMSKSGSGGSGGVNATFPPPLPPPPPASATTSGVHERSSSSASSPPRAPDLASENEQLKKDNHTLSAELAQARRHCEELLGFLSRFLDVRQLDLRLLMQEDVRAGASDDGAQRRAHAVASQLERGGGEEGKSVKLFGVLLKDAARKRGRCEEAAASERPIKMIRVGEPWVGVPSSGPGRCGGEN

SEQ ID NO: 39 - DNA, 4118 bp, Zea mays
  misc_difference 1538..2037: note = n is a, c, g, or t
  misc_difference 2846..3345: note = n is a, c, g, or t
  source 1..4118: mol_type = unassigned DNA
aggcgatgagcgcctctcgcgacgccagggcggagagctagccggccggagcccacacgcagctggaagcaccagaccgatcgtgccggccgagcggcggcgcaggcgcacaggcgcttacatgggagtagaggcgggcgggtgcgggcggagggcggtcgtcaccgggttctacgtctggggctgggagttcctcaccgccctccttctcttctcggccgccgtcgccgccgtagactcctactagcaagctacctaccttctttctttcattcccttaggtagctcagccgtacacacaacaacacacaagtcatcagttactagctagttagtagcctacacaacacatacatacatacaaaggtgagtgaggttcgcgtgcaagcagagccaatcgtgccgatcgagctatatatatacatagccggcggcgagagatgggagaagcggccgcggccgtggcggcgtcgaagaggggcggcgggccggcgccgttcctgaccaagacgcaccagatggtggaggagcggggcacggacgaggtgatctcgtgggcggagcagggccgctccttcgtggtgtggaagcccgtggagctggcgcgcgacctcctcccgctccacttcaagcactgcaacttctcctccttcgtccgccagctcaacacctacgtgagtacactacgccgccgctccggccatcatctcttctactacgatcgatgcacgaatcacgatctatataatatatcacctgttgtcgtcgttgagtgattgcaaaacgcatacacttggtttcagtattaaattaatcagctagctagatgatcgttctctgctctatgatctgttgttgttttcgtctgtgctcgataattaagctatgttatgtggtcgacgagcgagccctctagaaaccttccagcccgtcttccaaggagtatatgcgtgtcacggttcgtaggaaggagtgcgtcagtcatgacacatctctaccaccctttaattcctttcacacgcaaagcatgcttgtcgtttctgagctagctgaagaggaatgacctgcgataagacttgaagattagggtgccggtgcgggtctgaaattgcatctgtggtatgatcgtggtttagatagacttcacggatacgatcgcaagggagtttttttttctctacatgatctaaactgccatctctaactactatatatgtatagagcaaggtgcacggggaaattgaataagcaaagcaaatcaggtcggttgacacgccacggtattgtagtggcaacagaagcatgatattctatggaacagttaagaagcttcgtatggacgagcttttcagggctagctcttctgcgtggcctacaagaaggttaatttagctaggaattggatgctattagctgagcaagcaatatagggggtgtttgaatgcactagaactaatagttagttggcttaaacttgttagtagaattagctagctaacaaataactacctaactattaactaatttaccaaaaatannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaatttaccaaaaatagctaatagctgaactattagctagggtgtttggatgtctcaactaattctagccactaactattatctctagtgcattcaaacacccccataatcatccaaggcatccagcaagtatactaatcttttgttgccttccatctgttagctgggatactaaatattttgttggcttccatctgtttgtagtttctccaacagcttcataatttgtttttttttggctggatagtcttctccaaaatagcttcatggtaattggtaaagcttcttctttttttttctctctctcaatcgccaaaaggaaagcgatgtagggatacgaatagctgaaacgagtagcttattctagcgcagtcaaagattcacactgacattgggttcgttctcactgaaccttaatctattaatcagagggagagagagctagcttctctatcaatgtgtgtgaacagctaaggcgttatctgaccatgtgagcgacgtatggtggtcaaagtagacaggcctgacgtgttcatttcggcgtttgtttagggactggctgaataggacactgtgtcgaatgcagctcttgttctttttgccgcattggacacttacgtcgacggcgaccatcgcgcatgcgcatccatatccatgcagggtttccggaaggtggtgccggaccggtgggagttcgcgaacgacaacttccgtcgaggcgagcagggtctcctgtccggcatccgccgccgcaagtcaacggcgctgcagatgtccaagtccggatccggcggcagcggcggcgtgaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncggcgtcgtcgccaccgcgggcgcccgacctggccagcgagaacgagcagctcaagaaggacaaccacacgctgtccgtcgagctggcgcaggcgcgccggcactgcgaggagctcctgggcttcctctcgcgcttcctcgacgtccggcagctcgacctccggctgctcatgcaggaggacgtgcgagcgggggcaagcgacgacggcgcacagcgccgcgcgcacgcagtggccagccagctggagcgcggcggcggcgaggaggggaagagcgtgaagctgttcggcgtactcttaaaggacgccgcgaggaagaggggccggtgcgaggaagcggcggccagcgagcggcccatcaagatgatcagggtcggcgagccgtgggtcggcgtcccgtcgtcgggcccgggccggtgcggcggcgagaattaactgtcatccaatgtgaggttgatgacaaggacagtttcatccatcatatcgagcaagtaacaaagccagtgctgtggtaaaactgcaaagacagaacacaggacacaggagaaatataggcgtaagcatgttaattaagaattaattatatatgggatgcttttgaagtagcaagattggaagtagagataagtaaaacgggctagaagcagcgcccatgtgttcagaatggaaaattagcgtttccgtgtgtgtgttaagaaaaacttatatgcgctttctgcgagcacggttgattcttaagagcgacagcaaatgaaaggtgtattattaattgaa

SEQ ID NO: 40 - DNA, 762 bp, synthetic construct
  misc_feature 1..762: note = cDNA of ZmHsftf21 derived from PH207
  source 1..762: mol_type = other DNA
atgggagaagcggccgcggccgtggcggcgtcgaagaggggcggcgggccggcgccgttcctgaccaagacgcaccagatggtggaggagcggggcacggacgaggtgatctcgtgggcggagcagggccgctccttcgtggtgtggaagcccgtggagctggcgcgcgacctcctcccgctccacttcaagcactgcaacttctcctccttcgtccgccagctcaacacctacggtttccggaaggtggtgccggaccggtgggagttcgcgaacgacaacttccgtcgaggcgagcagggtctcctgtccggcatccgccgccgcaagtcaacggcgctgcagatgtccaagtccggatccggcggcagcggcggcctcaagaaggacaaccacacgctgtccgtcgagctggcgcaggcgcgccggcactgcgaggagctcctgggcttcctctcgcgcttcctcgacgtccggcagctcgacctccggctgctcatgcaggaggacgtgcgagcgggggcaagcgacgacggcgcacagcgccgcgcgcacgcagtggccagccagctggagcgcggcggcggcgaggaggggaagagcgtgaagctgttcggcgtactcttaaaggacgccgcgaggaagaggggccggtgcgaggaagcggcggccagcgagcggcccatcaagatgatcagggtcggcgagccgtgggtcggcgtcccgtcgtcgggcccgggccggtgcggcggcgagaattaa

SEQ ID NO: 41 - protein, 253 aa, Zea mays
MGEAAAAVAASKRGGGPAPFLTKTHQMVEERGTDEVISWAEQGRSFVVWKPVELARDLLPLHFKHCNFSSFVRQLNTYGFRKVVPDRWEFANDNFRRGEQGLLSGIRRRKSTALQMSKSGSGGSGGLKKDNHTLSVELAQARRHCEELLGFLSRFLDVRQLDLRLLMQEDVRAGASDDGAQRRAHAVASQLERGGGEEGKSVKLFGVLLKDAARKRGRCEEAAASERPIKMIRVGEPWVGVPSSGPGRCGGEN

SEQ ID NO: 42 - DNA, 5702 bp, Zea mays
  source 1..5702: mol_type = unassigned DNA
caacggctgatgaaagtaactactagaagttagtgataagttacgataattcaaagtagctagtacgtcagcttattattcgatctgactgcaagcatcatcgatatcgacggcttgcacacacggtagctagtttccttttttttttcacttttcgttttcaaagtccaagagttttaaatttgccgcagcgaagtttggctggcgcggctgttgcgcgtacgtgtagggaaagggaagggatcagtcatcagtgagagcactcacgcgcaggcgggcgcggcttcttcggggtccgcggaagcgagatgtggacaaatcgggggtgtgccgcaccgcagtggagtgcgacgagcgctccgagcacaagtccgcgctcgcgcgcgcattttccacgcgcctttgggtggtttactttctctcccggcgacggcgaggcaggcgcccgccagcgtcacaggtggtgacgaggcattccggtgccgaggaggatccaaaggacagtcggttcgtcctggcgcggtcgagacgggccgggccctcctccctcctgtgcgtgggagccagccagccagccaggagcggcgggccccgcttgggcgagcgacgaattttcgggcgctttgactcggctcggctcacggctcctggatattggacgacaaagcggtggaagcttcttatttggaccggccgcgggccggctgcaaggaagagcggctgaaaggggtgggcgagctgactgctgagcatacgtacccgcgcgaagaagcagacggaggtcatcacgctacccgcgcgtggccagtaccagacagactcctacctacactcagaaagcaagaagcccaacgccgaaagcaaccaccgcgctggtctctcgcctgtgccgccctcgatcgcgcgtgaagagaagcccctcacttccgtcctcctcctgtcctgtccagctaccccggccccgaccccgataaagcccgccctttaaatcggcggatcgaggcgatgagcgcctctcgcgacgccagggcggagagctagccggccgggagcccacacgcagctggaagcaccagaccgatcgtgccggccgagcggcggcgcaggcgcaggcgcttacatgggagtagaggcgggcgggtgcgggcggagggcggtcgtcaccgggttctacgtctggggctgggagttcctcaccgccctccttctcttctcggccgccgtcgccgccgcagactcctactagcaagctaccaaccttctttctttcattcccttaggtagctcagccgtacacacaacaacacacaagtcatcagttactagctagttagtagcctatacaacacatacatacatacaaaggtgagtgaggttcgcgtgcaagcagagccaatcgtgccgatcgagctatatacatagccggcggcgagggatgggagaagcggccgcggccgtggcggcgtcgaagaggggcggcgggccggcgccgttcctgaccaagacgcaccagatggtggaggagcggggcacggacgaggtgatctcgtgggcggagcagggccgctccttcgtggtgtggaagcccgtggagctggcgcgcgacctcctcccgctccacttcaagcactgcaacttctcctccttcgtccgccagctcaacacctacgtgagtacactacgccgccgctccggccatcatctcttctactacgatcgatgcaatatatcacctgtcgtcgtcgtttagtgattgcaaaacacatacacttggtttccgtattaaattaatcagctagctagctagatgatcgttctctgctctatgatctgttagttctgaagcatgttgttgttttcgtctgtgctcgataaattaagctatgttatgtggtcgacgagcgagccttccaggcagctaccgtaccgtcttccaaggagtatatgcgtgtgagcgtgtcacggttcgtaggaaggagtgcgtcagtcatgacacatctctaccaccctttaattcctttcccacgcaaagcatgcttgtcgtttcagagctagctgaagaggaatgacctgcgataacacttgaagattagggtgccggtgcgggtctgaaattacacctgtgggtacgatcgtgatttagatagacgacttcacggatgtgattacaggagtttttttttttctctacctgatctaaggccctgtttgggaacacagttttttcaaactgcagtttttcaaatactaaagtatactttagtcatgacattactacagtttacaatgcttcagttttcgaatacaacagtattcaatacatcaaggtgtttgggaaaaactttggttgagaccaatcagccagagcgggaccaagctggcactctctttacagagaaaaactttggctgagaccaaagtttccaaaactgcaaaacaagtgcagtatttgcaatactacagtttagtatacagagatttcagatgagtttccaaacacctcaaagtatataataccacagtattgctcaatactacagtattgcttcaatactgcagaaaaactttgttcccaaacaccccctaaactgccatctctaactactatatatgtatagagcaaggtgcacggggaaattgaataagcaaagcaaatcaggtcggttgacacgccacggtattgtagtggcgacagaagcatggtattctatggaacagttaaggccctgtttgggaacaaagtttttgaaaaccacagtttttgaaatactatactatactttagttatgacaataccgtagtttataataccgcagttttgaaaactgaggtccagagctaagtttagaatgccttaaaacaactatagtatttgcaatacttcagttttgaaaacagagattttacctagcttgccaaacaccattatgtatataatactgcagtatttgagaatactgcagtattcttccaaaactgcagaaaaactttgttcccaaacaccccctaagaagcttcggatggacgagcttttcagggctagctcttctgcgtggcctacaagaaggttaatttagctaggaattggatgctattagctgagcaagcaatataatcatccaaggcatccagcaagtatactaatcttttgttgcctcttccatctattagctgggatacgaaatcgctcaagaaattgacttggaagttaggatgatgatttaggccctgtttgtagtttctccaacagctagcttcataatttgtttttgttttttggctggatagtattttccaaaatagcttcatggtatttggtaaagcttcttctttttttctctctctcaagccaaaggaaagtgatgcagggatacgaatagctgaaacacgagtagcttattctagcgcagtcaaagattcacactgacttgggttcgttctcactgaaccttaatctattaatcagagggagagagagctagcttctctaaatcaatgtgtgaacagctataaggcgttatctgaccatgtgagcgacgtatggtggtcaaagtagacaggcctgacgtgttcatttcggcgtttgtttagggactggctgaataggacactgtgtcgaatgcagctcttgttctttttgccgcattggatacttacgtcgacggcgaccatggcgcatgcgcatccatatccatgcagggtttccgaaaggtggtgccggaccggtgggagttcgcgaacgacaacttccgtcgaggcgagcagggtctcctgtccggcatccgccgccgcaagtcaacggcgctgcagatgtccaagtccggatccggcggcagcggcggcgtgaacgccacgttccccccgcctctgccccctccgcctcccgcgtcggccaccacgtccggcgtccacgagcgcagctcgtcgtcggcgtcgtcgccaccgcgggcgcccgacctggccagcgagaacgagcagctcaagaaggacaaccacacgctgtccgccgagctggcgcaggcgcgccggcactgcgaggagctcctgggcttcctctcgcgcttcctcgacgtccggcagctcgacctccggctgctcatgcaggaggacgtgcgagcgggggcaagcgacgacggcgcacagcgccgcgcgcacgcagtggccagccagctggagcgcggcggcggcgaggaggggaagagcgtgaagctgttcggcgtactcttaaaggacgccgcgaggaagaggggccggtgcgaggaagcggcggccagcgagcggcccatcaagatgatcagggtcggcgagccgtgggtcggcgtcccgtcgtcgggcccgggccggtgcggcggcgagaattaactgtcatccaatgtgaggttgatgacaaggacagtttcatccatcatatcgagcaagtaacaaagccagtgctgtggtaaaactgcaaagacagaacacaggacacaggagaaatataggcgtaagcatgttaattaagaattaattatatatgggatgcttttgaagtagcaagattggaagtagagataagtaaaacgggctagaagcagcgcccatgtgttcagaatggaaaattagcgtttccgtgtgtgtgttaagaaaaacttatatgcgctttctgcgagcacggttgattcttaagagcgacagcaaatgaaaggtgtattattaattgaaggtcacttgaccacaaatattacctatctcatcatttcgttatggccttcacaggacgaggaagaaagagaaggatagactgtagagttctgtaaaagattctctaaatcaataatttaggtaattaatctaaaaacttctagtctcaacaactctttatatgaactttctaaatatagctactccccatctaatctcatttctatatacatttgacaaccatttaccaactccataaacaaaaaaataatagttgcattaacgtaggtaatgaaagtgtgtgttgacatttatgacttattttttaatgtgaatagatttaaagtaaggccctgtttgtttcaacttatagattatataatctagattatagtttagattatataatctggattatttgctctggattaaataagctaggtgctgctgtttgttagctcagattatttggactcggcttattattcatatgcatacaaatacaataatacacttgattgttttaattgtctggtgggtgagaacgcttatggataggtggatggcaattggaagtaattttaatcaacttgccatgggtagtgggtctttcataaaaaataagctgaaataagcaccctttgatgagcttataggattatcataatctcaagtgctagattatataatctcataagataagttacttgtttgtttcctcactagcttatttacattggattatataatctatatagattataatctcaaacaaacagggcctaaaactacaacctatatttagagagctattggagaactcatatttttttactcccaaacttatttagcaactacttaaatcaatgatttagagagctaaaatttatataactattggagctgctctaaagactcctaatgacatttaacaaattataaaatttcatattttttagttttaatagatttgtacattttaaccaaagcaatatgacattcatatatcaataatataatg

SEQ ID NO: 43 - DNA, 6118 bp, Zea mays
  misc_difference 2538..3037: note = n is a, c, g, or t
  misc_difference 3846..4345: note = n is a, c, g, or t
  misc_difference 5278..5777: note = n is a, c, g, or t
  source 1..6118: mol_type = unassigned DNA
ttttggcacggagaagaaatgaaatcaacggctgatgaaagtaactactagaagttagtgataagttacgataattcaaagtagctagtacgtcagcttattattcgatctgactgcaagcatcatcgatatcgacggcttgcacacacggtagctagtttccttttttttcacttttcgttttcaaagtccaagagttttaaatttgccgcagcgaagtttggctggcgcggctgttgcgcgtacgtgtagggaaagggaagggatcagtcatcagtgagagcactcacgcgcaggcgggcgcggcttcttcggggtccgcggaagcgagatgtggacaaatcgggggtgtgccgcaccgcagtggagtgcgacgagcgctccgagcacaagtccgcgctcgcgcgcgcattttccacgcgcctttgggtggtttactttctctcccggcgacggcgaggcaggcgcccgccagcgtcacaggtggtgacgaggcattccggtgccgaggaggacccaaaggacagtcggttcgtcctggcgcggtcgagacgggccgggccctcctccctcctgtgcgtgggagccagccagccagccaggagcggcgggccccgcttggtcgagcgacgaattttcgggcgctttgactcggctcggctcacggctcctggatattggacgacaaagcggtggaagcttcttatttggaccggccggctgcaagaaagagcggctgaaaggggtgggcgagctgagagcagacgtacccgcgcgaagaaacagacggaggtcatcacgctacccgcgcgtggccagtaccagacagactcctacactcagaaagcaagaagcccaacgccgaaagcaaccaccgcgctggtctctcgcctgtgcctcgatcgcgcgtgaagagaagccccctcagttccgtcctcctcctgtcctgtccagctacccccgaggccccgataagcccgccctttaaatcggcggatcgaggcgatgagcgcctctcgcgacgccagggcggagagctagccggccggagcccacacgcagctggaagcaccagaccgatcgtgccggccgagcggcggcgcaggcgcacaggcgcttacatgggagtagaggcgggcgggtgcgggcggagggcggtcgtcaccgggttctacgtctggggctgggagttcctcaccgccctccttctcttctcggccgccgtcgccgccgtagactcctactagcaagctacctaccttctttctttcattcccttaggtagctcagccgtacacacaacaacacacaagtcatcagttactagctagttagtagcctacacaacacatacatacatacaaaggtgagtgaggttcgcgtgcaagcagagccaatcgtgccgatcgagctatatatatacatagccggcggcgagagatgggagaagcggccgcggccgtggcggcgtcgaagaggggcggcgggccggcgccgttcctgaccaagacgcaccagatggtggaggagcggggcacggacgaggtgatctcgtgggcggagcagggccgctccttcgtggtgtggaagcccgtggagctggcgcgcgacctcctcccgctccacttcaagcactgcaacttctcctccttcgtccgccagctcaacacctacgtgagtacactacgccgccgctccggccatcatctcttctactacgatcgatgcacgaatcacgatctatataatatatcacctgttgtcgtcgttgagtgattgcaaaacgcatacacttggtttcagtattaaattaatcagctagctagatgatcgttctctgctctatgatctgttgttgttttcgtctgtgctcgataattaagctatgttatgtggtcgacgagcgagccctctagaaaccttccagcccgtcttccaaggagtatatgcgtgtcacggttcgtaggaaggagtgcgtcagtcatgacacatctctaccaccctttaattcctttcacacgcaaagcatgcttgtcgtttctgagctagctgaagaggaatgacctgcgataagacttgaagattagggtgccggtgcgggtctgaaattgcatctgtggtatgatcgtggtttagatagacttcacggatacgatcgcaagggagtttttttttctctacatgatctaaactgccatctctaactactatatatgtatagagcaaggtgcacggggaaattgaataagcaaagcaaatcaggtcggttgacacgccacggtattgtagtggcaacagaagcatgatattctatggaacagttaagaagcttcgtatggacgagcttttcagggctagctcttctgcgtggcctacaagaaggttaatttagctaggaattggatgctattagctgagcaagcaatatagggggtgtttgaatgcactagaactaatagttagttggcttaaacttgttagtagaattagctagctaacaaataactacctaactattaactaatttaccaaaaatannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaatttaccaaaaatagctaatagctgaactattagctagggtgtttggatgtctcaactaattctagccactaactattatctctagtgcattcaaacacccccataatcatccaaggcatccagcaagtatactaatcttttgttgccttccatctgttagctgggatactaaatattttgttggcttccatctgtttgtagtttctccaacagcttcataatttgtttttttttggctggatagtcttctccaaaatagcttcatggtaattggtaaagcttcttctttttttttctctctctcaatcgccaaaaggaaagcgatgtagggatacgaatagctgaaacgagtagcttattctagcgcagtcaaagattcacactgacattgggttcgttctcactgaaccttaatctattaatcagagggagagagagctagcttctctatcaatgtgtgtgaacagctaaggcgttatctgaccatgtgagcgacgtatggtggtcaaagtagacaggcctgacgtgttcatttcggcgtttgtttagggactggctgaataggacactgtgtcgaatgcagctcttgttctttttgccgcattggacacttacgtcgacggcgaccatcgcgcatgcgcatccatatccatgcagggtttccggaaggtggtgccggaccggtgggagttcgcgaacgacaacttccgtcgaggcgagcagggtctcctgtccggcatccgccgccgcaagtcaacggcgctgcagatgtccaagtccggatccggcggcagcggcggcgtgaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncggcgtcgtcgccaccgcgggcgcccgacctggccagcgagaacgagcagctcaagaaggacaaccacacgctgtccgtcgagctggcgcaggcgcgccggcactgcgaggagctcctgggcttcctctcgcgcttcctcgacgtccggcagctcgacctccggctgctcatgcaggaggacgtgcgagcgggggcaagcgacgacggcgcacagcgccgcgcgcacgcagtggccagccagctggagcgcggcggcggcgaggaggggaagagcgtgaagctgttcggcgtactcttaaaggacgccgcgaggaagaggggccggtgcgaggaagcggcggccagcgagcggcccatcaagatgatcagggtcggcgagccgtgggtcggcgtcccgtcgtcgggcccgggccggtgcggcggcgagaattaactgtcatccaatgtgaggttgatgacaaggacagtttcatccatcatatcgagcaagtaacaaagccagtgctgtggtaaaactgcaaagacagaacacaggacacaggagaaatataggcgtaagcatgttaattaagaattaattatatatgggatgcttttgaagtagcaagattggaagtagagataagtaaaacgggctagaagcagcgcccatgtgttcagaatggaaaattagcgtttccgtgtgtgtgttaagaaaaacttatatgcgctttctgcgagcacggttgattcttaagagcgacagcaaatgaaaggtgtattattaattgaaggtcacttgaccacaaatattacctatctcatcatttcgttatggcctctcggcaaagactattttacactcggcaaagcctttgccgagtgtaatactcggcaaagaacactcggcaaagatttcatcggcaaagggttctttgccgagtgtttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctcggcaaagatttcatcggcaaagggttctttgccgagtgtttttttcggacactcggcaaaagcactcagcaaagaaaaacactcggcaaattaagaatcgaaaaaaaattaaaaaaaacagcaaaacatttttttaaattataggaacaactctccaatcctacatattaccttatccgttgccgtatcatttttcactattattttgaatcaaatttagatactttgtaaatggtgagattcgaactcgtaacctctctctcgcgcataccctcctataccactacaccactacatcaattatgtctatactacgttttcattccccatgtactata

SEQ ID NO: 44 - DNA, 101 bp, synthetic construct
  misc_feature 1..101: note = KASP marker sequence 1
  source 1..101: mol_type = other DNA
aggtgaggtgagtggatgacatgatgaatgatgtctattttggttttcccrgtgtttcggttgttgcagtgtaaaaaccgaacccgacatagtagacctaa

SEQ ID NO: 45 - DNA, 101 bp, synthetic construct
  misc_feature 1..101: note = KASP marker sequence 2
  source 1..101: mol_type = other DNA
aaccattcaaaacatggtaaaccttacacacaataaccggcaagacaggamaaaggagtagcctacagcatcatgaaaccataaatacagagttagctaat

SEQ ID NO: 46 - DNA, 101 bp, synthetic construct
  misc_feature 1..101: note = KASP marker sequence 3
  source 1..101: mol_type = other DNA
ggctcatagctgcagcacgccacacatgaactgtggcacaccatatctacytgttaatgtcgctgttgggtgtgccaaaactgcagcaccgctgtgtaatt

SEQ ID NO: 47 - DNA, 101 bp, synthetic construct
  misc_feature 1..101: note = KASP marker sequence 4
  source 1..101: mol_type = other DNA
gtacagtacaggtacaaaactcacataagtagcagccactatacatacaartacaacgcgaacttaaactgaacagcagtagcattttccactcgtgtatg

SEQ ID NO: 48 - DNA, 121 bp, synthetic construct
  misc_feature 1..121: note = KASP marker sequence 5
  misc_difference 18: note = n is a, c, g, or t
  source 1..121: mol_type = other DNA
atctgcaactgtattcangtcctttgttgctttggcctctggcgcagaagataaactccarccgttcttggaagcactcgttgctggcctaattaatgacagattgtcctttcgatcaaag

SEQ ID NO: 49 - DNA, 101 bp, synthetic construct
  misc_feature 1..101: note = KASP marker sequence 6
  source 1..101: mol_type = other DNA
catccctacataaggaagcaattagcaactgataaccacaggttggcgatmttaactctgctactaaaatctatctcatcatctaggccttgcttctagcc

SEQ ID NO: 50 - DNA, 101 bp, synthetic construct
  misc_feature 1..101: note = KASP marker sequence 7 = marker A
  source 1..101: mol_type = other DNA
ctctgcttgcacgcgaacctcactcacctttgtatgtatgtatgtgttgtytaggctactaactagctagtaactgatgacttgtgtgttgttgtgtgtac

SEQ ID NO: 51 - DNA, 121 bp, synthetic construct
  misc_feature 1..121: note = KASP marker sequence 8a / marker C
  source 1..121: mol_type = other DNA
acctgcacacaaggcatgcatggtcgttccgttcgaatcattagcagcttgtgaacaaggrggcggatccagaacgaaggacgaacacgctgcccgcatgagtagtagtagttgcctacgg

SEQ ID NO: 52 - DNA, 121 bp, synthetic construct
  misc_feature 1..121: note = KASP marker sequence 8b / marker B
  source 1..121: mol_type = other DNA
tccgagctaggcatatgaaagcatagatcaacactgtgaagccgcaatgatgactgatgarcccatgaagccacatcaatagatgaatattgagcccatttgcattttgtactttgttttg

SEQ ID NO: 53 - DNA, 121 bp, synthetic construct
  misc_feature 1..121: note = KASP marker sequence 9 / marker D
  source 1..121: mol_type = other DNA
tgatacaacaaatggtacaaatgttacaatagcaaggtaatgccaaatgtggcgacaattrcacgcattacgaccgatcctgcagcttattcctatttttttcttaatagtttcaaccgga

SEQ ID NO: 54 - DNA, 121 bp, synthetic construct
  misc_feature 1..121: note = KASP marker sequence 10 / marker E
  source 1..121: mol_type = other DNA
tcagaatcttctttcctatataggcacttcactggctggctcttctaggggagaaagaaaycactcatgccactacaccgatttttaatatctttctaaatgcctgtggtagagcaaatct

SEQ ID NO: 55 - DNA, 71 bp, synthetic construct
  misc_feature 1..71: note = KASP marker sequence 11 / marker F
  source 1..71: mol_type = other DNA
tcttctccaaatccaagtccaagtctaagtccaagyccaagaaggagaagtcgaagcctgatggtccaaac

SEQ ID NO: 56 - DNA, 121 bp, synthetic construct
  misc_feature 1..121: note = KASP marker sequence 12
  misc_difference 32: note = n is a, c, g, or t
  misc_difference 37: note = n is a, c, g, or t
  misc_difference 45: note = n is a, c, g, or t
  source 1..121: mol_type = other DNA
tggatgcctgattactcctctacctgctcgantcgtngcctagangtttctgtggtcctgytcaggcacacaatatgcaatagctataggattaacaaacaaataacaacaagtctaacaa

SEQ ID NO: 57 - DNA, 121 bp, synthetic construct; identical to SEQ ID NO: 56
  misc_feature 1..121: note = KASP marker sequence 13
  misc_difference 32: note = n is a, c, g, or t
  misc_difference 37: note = n is a, c, g, or t
  misc_difference 45: note = n is a, c, g, or t
  source 1..121: mol_type = other DNA

SEQ ID NO: 58 - DNA, 121 bp, synthetic construct; identical to SEQ ID NO: 56
  misc_feature 1..121: note = KASP marker sequence 14
  misc_difference 32: note = n is a, c, g, or t
  misc_difference 37: note = n is a, c, g, or t
  misc_difference 45: note = n is a, c, g, or t
  source 1..121: mol_type = other DNA

SEQ ID NO: 59 - DNA, 101 bp, synthetic construct
  misc_feature 1..101: note = KASP marker sequence 15
  source 1..101: mol_type = other DNA
aaaaattttaggtcctgtgacctgtattacactcaagaagctatcagcaartacctggtagctctgccaataacttcaccattagctaggtccttgaggat

SEQ ID NO: 60 - DNA, 121 bp, synthetic construct
  misc_feature 1..121: note = KASP marker sequence 16
  source 1..121: mol_type = other DNA
agagcccccacctccagttctgctggcagtggcttgaggcgaatctggaatcagaccacaratgacagcttctgcagtatctctatacattttcaacccctcattttcggcattagatatc

SEQ ID NO: 61 - DNA, 101 bp, synthetic construct
  misc_feature 1..101: note = KASP marker sequence 17
  source 1..101: mol_type = other DNA
ccgcgatgtactgcagctagtaaatcagcgaggggcagaggggagacccargtcggtatgcatctgtcaatataatgcaggggttggaactagaagagagg

SEQ ID NO: 62 - DNA, 121 bp, synthetic construct
  misc_feature 1..121: note = KASP marker sequence 18
  misc_difference 22: note = n is a, c, g, or t
  source 1..121: mol_type = other DNA
cagcaatctaactctcatgtanagcattcaaaaattggatctggtgagggtgattgtgtcrctaccccagcacccccatccattgaagcttcacctgcacttcctgtcgattgcgatgatg

(19) United States
(12) Patent Application Publication
(10) Pub. No.: US 2025/0169413 A1
(43) Pub. Date: May 29, 2025

(54) DROUGHT TOLERANCE IN CORN

(71) Applicants: KWS SAAT SE & Co. KGaA, Einbeck (DE); TECHNISCHE UNIVERSITAT MUNCHEN, Munich (DE)

(72) Inventors: Claude URBANY, Einbeck (DE); Milena OUZUNOVA, Göttingen (DE); Thomas PRESTERL, Einbeck (DE); Daniela SCHEUERMANN, Einbeck (DE); Chris-Carolin SCHÖN, Munich (DE); Svenja ALTER, March (DE); Viktoriya AVRAMOVA, Freising (DE); Eva BAUER, Zolling (DE); Sebastian GRESSET, Garching (DE)

(73) Assignees: KWS SAAT SE & Co. KGaA, Einbeck (DE); TECHNISCHE UNIVERSITAT MUNCHEN, Munich (DE)

(21) Appl. No.: 19/049,699

(22) Filed: Feb. 10, 2025

Related U.S. Application Data
(62) Division of application No. 17/610,529, filed on Nov. 11, 2021.

(30) Foreign Application Priority Data
May 13, 2019 (EP) 19174242.8
Oct. 4, 2019 (EP) 19201403.3
Mar. 17, 2020 (EP) 20163676.8

(51) Int. Cl. A01H 1/00; C12N 15/82; C12Q 1/6895

(52) U.S. Cl. CPC A01H 1/1225; C12N 15/8273; C12Q 1/6895; C12Q 2600/13; C12Q 2600/156

(57) ABSTRACT

The present invention relates to a QTL allele in maize associated with drought resistance and carbon isotope composition as well as specific marker alleles associated with the QTL allele. The present invention further relates methods for identifying maize plants based on screening for the presence of the QTL allele or marker alleles. The invention also relates to methods for modifying drought resistance and carbon isotope composition in maize plants.

### CROSS-REFERENCE TO RELATED APPLICATIONS

This application is a divisional of U.S. application Ser. No. 17/610,529, filed Nov. 11, 2021, which is a U.S. National Phase of International Patent Application No. PCT/EP2020/063317, filed on May 13, 2020, which claims priority to EP Application No. 20163676.8, filed Mar. 17, 2020, EP Application No. 19201403.3, filed Oct. 4, 2019, and EP Application No. 19174242.8, filed May 13, 2019. The entire contents of these applications are incorporated herein by reference in their entirety.

### FIELD OF THE INVENTION

The invention relates to quantitative trait loci (QTL) and associated markers involved in and/or associated with drought tolerance, carbon isotope composition, stomatal parameters, and agronomic performance of plants and plant parts, such as maize. The invention further relates to uses of such QTL or markers for identification and/or selection purposes, as well as transgenic or non-transgenic plants.

### SEQUENCE LISTING

The instant application contains a Sequence Listing which has been submitted electronically in XML format and is hereby incorporated by reference in its entirety. Said XML copy, created on Jan. 22, 2025, is named 245761_000250_SL.xml and is 186,760 bytes in size.

### BACKGROUND OF THE INVENTION

Drought stress is one of the most severe natural limitations of productivity in agricultural systems around the world. With climate changes, crops will be subjected to more frequent episodes of drought and high temperature that impede growth and development at all plant stages (IPCC, 2014). Especially, when such conditions hit plant development before, during, and after flowering a reduction in plant performance and yield is almost certain. Breeding for drought tolerant crop varieties is an urgent priority to tackle the environmental challenges mentioned above and provide to the farmers crop plants for sustainable production systems.

Gresset et al. (2014. Stable carbon isotope discrimination is under genetic control in the C4 species maize with several genomic regions influencing trait expression. Plant Physiology, 164(1), 131-143) reported on the analysis of a proprietary maize (Zea mays L.) introgression library (IL) derived from two inbred lines of KWS SAAT SE, obtained from a European elite dent as recurrent parent (RP) and a flint line as donor parent (DP) with the purpose to reveal a potential genetic control of carbon isotope composition (δ13C). Highly heritable significant genetic variation for δ13C was detected under field and greenhouse conditions. From the evaluation of 77 IL lines the authors were able to identify 22 genomic regions affecting δ13C. Two target regions thereof located on chromosomes 6 and 7 seemed to be of particular relevance (FIG. 1A).

Carbon isotope composition can be used as proxy for inferring information about transpiration efficiency in C3 species (Farquhar et al., 1989. Carbon isotope discrimination and photosynthesis. Annual review of plant biology, 40(1), 503-537). Several studies in C4 species have shown negative correlations between δ13C and water use efficiency (WUE; Henderson et al., 1998. Correlation between carbon isotope discrimination and transpiration efficiency in lines of the C4 species Sorghum bicolor in the glasshouse and the field. Functional Plant Biology, 25(1), 111-123; Dercon et al., 2006. Differential 13 C isotopic discrimination in maize at varying water stress and at low to high nitrogen availability. Plant and Soil, 282(1-2), 313-326; Sharwood et al., 2014. Photosynthetic flexibility in maize exposed to salinity and shade. Journal of experimental botany, 65(13), 3715-3724.), which is defined as the amount of biomass or yield accumulated per unit of water used.

Avramova et al. (2019. Carbon isotope composition, water use efficiency, and drought sensitivity are controlled by a common genomic segment in maize. Theoretical and Applied Genetics, 132:53-63) analyzed further near isogenic lines of Gresset et al. 2014 carrying overlapping donor segments on chromosome 7. Two near isogenic lines, NIL A and NIL B were developed from crosses between lines from the introgression library. A genotypic analysis with the 600 k Axiom™ Maize Genotyping Array (Unterseer et al., 2014. A powerful tool for genome analysis in maize: development and evaluation of the high density 600 k SNP genotyping array. BMC Genomics, 15:823) showed that both NILs carry a genomic segment derived from DP on chromosome 7, which was shown to significantly increase kernel δ13C compared to RP. The authors hypothesized that the introgression segment on chr 7 (110.76-166.10 Mb) carried by NIL B (FIG. 1C) harbours several QTL that affect different traits and have a cumulative effect on individual traits. The latter can be inferred from NIL A (FIG. 1B) with a smaller segment on chr 7 than NIL B and a less pronounced effect on the measured parameters. Furthermore, NIL A carries a second large segment on chr 2, where a previously identified QTL for δ13C is located (Gresset et al. 2014), which might alter the effect of the introgression on chr 7.

From a study of Alvarez Prado et al. (2018. Phenomics allows identification of genomic regions affecting maize stomatal conductance with conditional effects of water deficit and evaporative demand. Plant, cell & environment, 41(2), 314-326.) three additional QTL affecting whole-plant stomatal conductance (two with positive and one with negative effect) have been identified in the same genomic region as Avramova et al. (124.35-160.14 Mb) on chromosome 7 in a maize diversity panel.

Even though regions on chromosome 7 in corn has been intensively studied in the light of affecting carbon isotope composition, stomatal parameters and agronomic performance, the focus was often more directed to phenotypical aspects and physiological parameters than on the genomic nature. Several QTL have been found, partly influencing drought tolerance positively, partly negatively. The interaction between these QTL is not well-studied and not fully understood yet. Furthermore, the genomic region investigated by Avramova et al. 2019 and presumably carrying several relevant QTL is with more than 20 Mb rather large and the availability of suitable molecular markers is very limited, that is why up to now this trait is not efficiently usable in breeding and plant development. There is a need for genomic characterization of small genomic regions or causative genes as well as molecular markers allowing to follow these genomic regions or genes during breeding processes and to introgress them into new elite line without possibly attached linkage drag.

It is therefore an objective of the present invention to address one or more of the shortcomings of the prior art. There is a persistent need for improving drought resistance of fodder crops, as well as the identification of plants, including particular plant parts or derivatives having altered drought resistance. In particular, it is an aim of the present invention to provide new major QTL for among others drought resistance and associated parameters, such as carbon isotope composition, stomatal parameters, and agronomic performance, and the causative gene(s) and the provision of markers which allow the economical use of these QTL in maize development and breeding.

### SUMMARY OF THE INVENTION

The present invention is based on the identification of a QTL contributing to genetic variation among others in stable carbon isotope composition, stomatal conductance and plant performance under drought.

The invention in particular relates to methods for identifying a maize plant or plant part, comprising screening for the presence of a QTL allele located on chromosome 7, wherein said QTL allele is located on a chromosomal interval comprising specific molecular markers. The QTL allele preferably comprises molecular markers A and/or B, wherein molecular markers A and B are SNPs (single nucleotide polymorphisms) which are respectively C corresponding to position 125861690 and A corresponding to position 126109267 or which are respectively T corresponding to position 125861690 and G corresponding to position 126109267, referenced to the B73 reference genome AGPv2. In certain embodiments, the QTL allele is flanked by molecular markers A and/or B. In certain embodiments, said QTL allele comprises molecular markers C, D, E, and/or F, wherein molecular markers C, D, E, and F are SNPs which are respectively A corresponding to position 125976029, A corresponding to position 127586792, C corresponding to position 129887276, and C corresponding to position 130881551, or which are respectively G corresponding to position 125976029, G corresponding to position 127586792, T corresponding to position 129887276, and T corresponding to position 130881551, referenced to the B73 reference genome AGPv2. In certain embodiments, said QTL allele is flanked by molecular markers A and/or F.

The invention further relates to the described markers or marker alleles and polynucleic acids useful for detection of the markers or marker alleles, such as primers and probes, and kits comprising such. The invention further relates to methods for modifying plant drought resistance or tolerance, in particular by naturally or artificially introducing in plants and/or selecting plants comprising the QTL (allele) and/or markers or marker alleles described herein, as well as modifying gene expression or gene activity of genes comprised in the QTL (allele) according to the invention as defined herein. The invention further relates to plants comprising the QTL (allele) and/or markers or marker alleles according to the invention as defined herein.

The invention in particular allows to use molecular markers to infer the genomic state of
i) a QTL of 5.02 Mb between the flanking markers 7 (125.861.690 bp) and 11 (130.881.551 bp) on chromosome 7 affecting δ13C and stomatal parameters,
ii) a truncated part of this QTL of 248 kb ranging from marker 7 (125.861.690 bp) to marker 8b (126.109.267 bp) with a specific effect on gas-exchange parameters,
and to select based on the genes mapping to the 5.02 Mb interval. The genotype/phenotype correlations of introgression lines with donor parent (DP) segments and recurrent parent (RP) allow to deduce and alter carbon isotope composition, reaction mode of stomatal parameters and expression of agronomic performance in germplasm. In this respect, under a mild stress scenario, the donor introgression can be used to keep stomatal conductance at elevated levels even under water stress. Thus, a prolonged photosynthesis and a slight growth advantage after recovery is realized that improves agronomics and yield. In addition, the information can also be used to introgress DP alleles to promote a faster drought response in drought-prone germplasm.

Generally, the invention allows to use the marker information to characterize material upon stomatal parameters, carbon isotope composition, water use efficiency and performance under drought. Correspondingly, using single marker information as well as binned information resulting in haplotypes is the basis for a fast, precise and improved classification of genetic material during a common selection process.

Finally, allelic variation at the candidate gene level can be used to improve the above-mentioned phenotypes by either modulating expression of candidate genes, modifying the molecular activity of such genes and gene products or generating any allelic versions derived from such genes.

The present invention is in particular captured by any one or any combination of one or more of the below numbered statements 1 to 25, with any other statement and/or embodiments.

[1]A method for identifying a maize plant or plant part, comprising screening for the presence of a QTL allele located on chromosome 7, wherein said QTL allele is located on a chromosomal interval comprising molecular markers (alleles) A and/or B, wherein molecular markers (alleles) A and B are SNPs which are respectively C corresponding to position 125861690 and A corresponding to position 126109267 or which are respectively T corresponding to position 125861690 and G corresponding to position 126109267, referenced to the B73 reference genome AGPv2.

[2] The method according to statement 1, wherein said QTL allele is flanked by molecular markers (alleles) A and/or B, preferably both, optionally wherein said QTL allele comprises molecular markers (alleles) A and/or B, preferably both.

[3] The method according to any of statements 1 to 2, wherein said QTL allele comprises molecular markers (alleles) C, D, E, and/or F, wherein molecular markers (alleles) C, D, E, and F are SNPs which are respectively A corresponding to position 125976029, A corresponding to position 127586792, C corresponding to position 129887276, and C corresponding to position 130881551, or which are respectively G corresponding to position 125976029, G corresponding to position 127586792, T corresponding to position 129887276, and T corresponding to position 130881551, referenced to the B73 reference genome AGPv2.

[4] The method according to statement 3, wherein said QTL allele is flanked by molecular markers A and/or F, preferably both, optionally wherein said QTL allele comprises molecular markers (alleles) A and/or F, preferably both.

[5] The method according to any of statements 1 to 4, wherein screening for the presence of said QTL allele comprises identifying any one or more of molecular markers A and B.

[6] The method according to any of statements 3 to 5, wherein screening for the presence of said QTL allele comprises identifying any one or more of molecular markers A, B, C, D, E, and F.

[7] The method according to any of statements 3 to 5, wherein screening for the presence of said QTL allele comprises determining the expression level, activity, and/or sequence of one or more gene located in the QTL as defined in any of statements 1 to [6]

[8]A method for identifying a maize plant or plant part, comprising determining the expression level, activity, and/or sequence of one or more gene located in the QTL as defined in any of statements 1 to 6.

[9] The method according to statement 7 or 8, further comprising comparing the expression level and/or activity of said one or more gene with a predetermined threshold.

[10] The method according to any of statements 7 to 9, further comprising comparing the expression level and/or activity of said one or more gene under control conditions and drought stress conditions.

[11]A method of modifying a maize plant, comprising altering the expression level and/or activity of one or more gene located in the QTL as defined in any of statements 1 to 6.

[12] The method according to any of statements 7 to 11, wherein said one or more gene is selected from Abh4, CSLE1, WEB1, RMZM2G397260, and Hsftf21, preferably Abh4.

[13] The method according to statement 12, wherein Abh4 is selected from
(i) a nucleotide sequence comprising or consisting of the sequence of SEQ ID NO: 9 or 18;
(ii) a nucleotide sequence having the cDNA of SEQ ID NO: 11, 14, 17, or 20;
(iii) a nucleotide sequence encoding for an amino acid sequence having the amino acid sequence of SEQ ID NO: 12, 15, or 21;
(iv) a nucleotide sequence having at least 60%, preferably at least 80%, more preferably at least 90%, even more preferably at least 95% identity to the sequence of SEQ ID NO: 9, 11, 14, 17, 18, or 20;
(v) a nucleotide sequence encoding for a polypeptide having at least 60%, preferably at least 80%, more preferably at least 90%, even more preferably at least 95% identity to the sequence of SEQ ID NO: 12, 15, or 21;
(vi) a nucleotide sequence hybridizing with the reverse complement of a nucleotide sequence as defined in (i), (ii) or (iii) under stringent hybridization conditions; and
(vii) a nucleotide sequence encoding a protein derived from the amino acid sequence encoded by the nucleotide sequence of (i) to (vi) by way of substitution, deletion and/or addition of one or more amino acid(s);

CSLE1 is selected from
(i) a nucleotide sequence comprising or consisting of the sequence of SEQ ID NO: 1 or 4;
(ii) a nucleotide sequence having the cDNA of SEQ ID NO: 2 or 5;
(iii) a nucleotide sequence encoding for an amino acid sequence having the amino acid sequence of SEQ ID NO: 3 or 6;
(iv) a nucleotide sequence having at least 60%, preferably at least 80%, more preferably at least 90%, even more preferably at least 95% identity to the sequence of SEQ ID NO: 1, 2, 4, or 5;
(v) a nucleotide sequence encoding for a polypeptide having at least 60%, preferably at least 80%, more preferably at least 90%, even more preferably at least 95% identity to the sequence of SEQ ID NO: 3 or 6;
(vi) a nucleotide sequence hybridizing with the reverse complement of a nucleotide sequence as defined in (i), (ii) or (iii) under stringent hybridization conditions; and
(vii) a nucleotide sequence encoding a protein derived from the amino acid sequence encoded by the nucleotide sequence of (i) to (vi) by way of substitution, deletion and/or addition of one or more amino acid(s);

WEB1 is selected from
(i) a nucleotide sequence comprising or consisting of the sequence of SEQ ID NO: 24 or 27;
(ii) a nucleotide sequence having the cDNA of SEQ ID NO: 25 or 28;
(iii) a nucleotide sequence encoding for an amino acid sequence having the amino acid sequence of SEQ ID NO: 26 or 29;
(iv) a nucleotide sequence having at least 60%, preferably at least 80%, more preferably at least 90%, even more preferably at least 95% identity to the sequence of SEQ ID NO: 24, 25, 27, or 28;
(v) a nucleotide sequence encoding for a polypeptide having at least 60%, preferably at least 80%, more preferably at least 90%, even more preferably at least 95% identity to the sequence of SEQ ID NO: 26 or 29;
(vi) a nucleotide sequence hybridizing with the reverse complement of a nucleotide sequence as defined in (i), (ii) or (iii) under stringent hybridization conditions; and
(vii) a nucleotide sequence encoding a protein derived from the amino acid sequence encoded by the nucleotide sequence of (i) to (vi) by way of substitution, deletion and/or addition of one or more amino acid(s);

GRMZM2G397260 is selected from
(i) a nucleotide sequence comprising or consisting of the sequence of SEQ ID NO: 32;
(ii) a nucleotide sequence having the cDNA of SEQ ID NO: 33;
(iii) a nucleotide sequence encoding for an amino acid sequence having the amino acid sequence of SEQ ID NO: 34;
(iv) a nucleotide sequence having at least 60%, preferably at least 80%, more preferably at least 90%, even more preferably at least 95% identity to the sequence of SEQ ID NO: 32 or 33;
(v) a nucleotide sequence encoding for a polypeptide having at least 60%, preferably at least 80%, more preferably at least 90%, even more preferably at least 95% identity to the sequence of SEQ ID NO: 34;
(vi) a nucleotide sequence hybridizing with the reverse complement of a nucleotide sequence as defined in (i), (ii) or (iii) under stringent hybridization conditions; and
(vii) a nucleotide sequence encoding a protein derived from the amino acid sequence encoded by the nucleotide sequence of (i) to (vi) by way of substitution, deletion and/or addition of one or more amino acid(s);

Hsftf21 is selected from
(i) a nucleotide sequence comprising or consisting of the sequence of SEQ ID NO: 36 or 39;
(ii) a nucleotide sequence having the cDNA of SEQ ID NO: 37 or 40;
(iii) a nucleotide sequence encoding for an amino acid sequence having the amino acid sequence of SEQ ID NO: 38 or 41;
(iv) a nucleotide sequence having at least 60%, preferably at least 80%, more preferably at least 90%, even more preferably at least 95% identity to the sequence of SEQ ID NO: 36, 37, 39, or 40;
(v) a nucleotide sequence encoding for a polypeptide having at least 60%, preferably at least 80%, more preferably at least 90%, even more preferably at least 95% identity to the sequence of SEQ ID NO: 38 or 41;
(vi) a nucleotide sequence hybridizing with the reverse complement of a nucleotide sequence as defined in (i), (ii) or (iii) under stringent hybridization conditions; and
(vii) a nucleotide sequence encoding a protein derived from the amino acid sequence encoded by the nucleotide sequence of (i) to (vi) by way of substitution, deletion and/or addition of one or more amino acid(s).

[14]A method for generating a maize plant, comprising introducing into the genome of a plant a QTL allele as defined in any of statements 1 to 6.

[15]A method for obtaining a maize plant part, comprising (a) providing a first maize plant having a QTL allele or one or more molecular marker as defined in any of statements 1 to 6, (b) crossing said first maize plant with a second maize plant, (c) selecting progeny plants having said QTL allele or said one or more molecular marker, and (d) harvesting said plant part from said progeny.

[16] The method according to any of statements 1 to 15, wherein said QTL is associated with drought resistance or tolerance and/or δ13C.

[17] The method according to any of statements 1 to 16, wherein said QTL affects stomatal parameters and/or gas-exchange parameters.

[18] The method according to any of statements 1 to 17, wherein said QTL affects (intrinsic or whole plant) water use efficiency, stomatal conductance, net CO2 assimilation rate, transpiration, stomatal density, (leaf) ABA content, sensitivity of (leaf) growth to drought, evaporative demand and/or soil water status and/or photosynthetic response.

[19]A maize plant or plant part comprising a QTL allele and/or one or more molecular marker as defined in any of statements 1 to 18.

[20] The plant or plant part according to statement 19, wherein said plant is derived from a plant comprising said QTL allele or marker alleles obtained by introgression.

[21] The plant or plant part according to statement 19 or 20, wherein the plant is transgenic or gene-edited.

[22] The method, plant or plant part according to any of the preceding statements, wherein said plant part is not propagation material.

[23] An isolated polynucleic acid specifically hybridising with a maize genomic nucleotide sequence comprising any one or more of molecular markers A, B, C, D, E, and F, or the complement or the reverse complement thereof.

[24] The isolated polynucleic acid according to statement 23 which is a primer or probe capable of specifically detecting the QTL allele or any one or more molecular markers as defined in any of statements 1 to 6.

[25] An isolated polynucleic acid comprising and/or flanked by any one or more of molecular markers A, B, C, D, E, or F.

### BRIEF DESCRIPTION OF THE FIGURES AND SEQUENCES

FIGS. 1A-1C. Graphical genotypes of IL-005 (FIG. 1A), NIL A (FIG. 1B) and NIL B (FIG. 1C). Chromosomes (Chr) and centromeres (centromer) with marker distribution and corresponding RP (black) and DP (grey) calls are shown. Physical coordinates relate to AGPv02. Detailed data are received from the 600K array.

FIG. 2. Overview about size and state of the chromosome 7 introgression in IL-005, NIL A and NIL B and the significant interval as reported in Gresset et al. (2014). The lower track gives the overall distribution of 600 markers (black bars) and gene models (gene) on maize AGPv02 chr 7. The size of the introgression (donor target) in ILs with number of markers at DP state (DP calls) is shown as well as the corresponding number of gene models within the introgression. The upper track gives an overview about the molecular state of the target reported in Gresset et al. (2014).

FIG. 3. Overview of the selection process of newly generated recombinants. KASP markers are shown by vertical orange lines and points with respective names. Possible recombination events that were detected during the screening are represented by black/grey stairs.

FIG. 4. Identified recombinants and molecular state of QTL. Recombinants are plotted with their corresponding name. Sequence intervals with size and state referring to homozygous RP (black) and homozygous DP (grey) are depicted. The target interval of 5.02 Mb is framed by two lines (arrows).

FIG. 5. Gene expression of ZmAbh4 (all transcripts togehter) and transcripts T01 and T03 separately in well-watered (control; C), drought-stressed (D) and re-watered plants (R). The gene expression was compared between the recurrent parent (genotype RP) and a near-isogenic line (genotype NIL B), carrying the donor parent allele of the gene. Two-way ANOVA was conducted to assess significant differences between genotypes, treatments and the interaction between them regarding the expression of all ZmAbh4 transcripts together and P-values are displayed under the first pannel. Nd: not detected

FIG. 6. Chemical reaction catalized by Abh4. The figure is taken from Saito et al. (2004). Arabidopsis CYP707As encode (+)-abscisic acid 8′-hydroxylase, a key enzyme in the oxidative catabolism of abscisic acid. Plant Physiol. 134 (4): 1439-1449. Arabidopsis CYP707As encode (+)-abscisic acid 8′-hydroxylase, a key enzyme in the oxidative catabolism of abscisic acid. Plant Physiol. 134 (4): 1439-1449.

FIG. 7. Ratio of products (PA phaseic acid, DPA dihydrophaseic acid) to substrate (ABA abscisic acid) of the reaction catalized by Abh4 for the recombinants from FIG. 4. Sequence intervals with size and state referring to homozygous RP (dark grey) and homozygous DP (light grey) are depicted. Diplayed are AGPv02 coordinates. The overlapping interval for recombinants with the same phenotype is framed. An LSD comparison between RP and each recombinant was conducted (N=10) and * P<0.05, ** P<0.01, *** P<0.001.

FIG. 8. Ratio of products (PA phaseic acid, DPA dihydrophaseic acid) to substrate (ABA abscisic acid) of the reaction catalized by Abh4 for TILLING lines carrying the mutations P377L (377mut) or G453E (453mut), and their respective wild types (377WT, 453WT) as well as heterozygous plants for the mutation G453E (453het) and the inbred line used for generating the mutants, PH207. N=7-12. * p<0.05

FIG. 9. Carbon isotope discrimination (Δ¹³C) of the last developed leaf of TILLING lines carrying the mutations P377L (377mut) or G453E (453mut), and their respective wild types (377WT, 453WT) as well as heterozygous plants for the mutation G453E (453het) and PH207. N=8-12

FIGS. 10A-10B. Stomatal conductance (gₛ) (FIG. 10A) and Instantaneous water use efficiency (iWUE) (FIG. 10B) measured for Mo17, B73, PH207 and three NILs with the background of Mo17 and introgressed segments originating from B73 on chromosome 7 (m031, m007, m046; Eichten et al. 2011). Color coding dependent on Abh4 allele carried by the line. N=10-11. Significant differences (p<0.05) are marked by discrete letters.

FIGS. 11A-11C. Ratio of products (PA phaseic acid, DPA dihydrophaseic acid) to substrate (ABA abscisic acid) of the reaction catalyzed by ZmAbh4 for PH207, B73 and two NILs with the background of B73 and introgressed segments originating from Mo17 on chromosome 7 (b004, b102; Eichten et al. 2011).). N=12 (FIG. 11A) Stomatal conductance (gs) (FIG. 11B) and Instantaneous water use efficiency (NWUE) (FIG. 10C) measured for B73, PH207 and the two NILs. N=13-14. Color coding dependent on Abh4 allele carried by the line. Significant differences (p<0.05) are marked by discrete letters.

FIG. 12. ABA and its catabolites PA, DPA and ABA-Glc in T1 generation of CRISPR/Cas9 mutants grown in the greenhouse. Concentrations in leaves of plants carrying two mutant copies of ZmAbh4 (mutant, n=3) compared to plants carrying two wildtype (WT, n=4) copies of ZmAbh4 (means±SD).

FIG. 13. Gas exchange measurements of leaf 6 (V6) of CRISPR/Cas9 mutants in T1 generation grown in the greenhouse. Wildtype line B104 (n=17), Wildtype siblings of the mutant plants (WTsib, n=5), plants showing a mutation in ZmAbh4, but not in ZmAbh1 (zmabh4, n=9) and plants showing a mutation in both genes, ZmAbh4 and ZmAbh1 (zmabh4 zmabh1, n=15), were measured. No multiple testing correction due to high heterogeneity in T1.

FIG. 14. Comparison of the near isogenic line B (NIL B) and nine recombinant NILs (D-L) to their recurrent parent (RP) in terms of whole plant water use efficiency (WUEₚₗₐₙₜ). Each NIL carries an introgression (marked with dark grey) from a flint donor parent in the genetic background of the dent RP (light grey). Starting with the same amount of soil and water in the pots, plants were subjected to progressive soil drying conditions. Water evaporation through the soil surface was prevented by plastic covering of the pots. Final dry biomass was measured at the end of the experiment when plants stopped growing and WUEₚₗₐₙₜ was calculated as the ratio between final dry biomass and consumed water. Data are means±standard error (n=10). Significant differences between RP and each of the NILs based on Dunnet's test are indicated with dark grey color of the bars (light grey bars do not differ significantly from RP). The black square frame indicates the target genomic region associated with the trait. Coordinates indicated in the last row are according to B73 v4 (maizegdb.org).

FIG. 15. Comparison of the near isogenic line B (NIL B) and nine recombinant NILs (D-L) to their recurrent parent (RP) in terms of intrinsic water use efficiency (iWUE). Each NIL carries an introgression (marked with dark grey) from a flint donor parent in the genetic background of the dent RP (light grey). Leaf gas-exhchange measurements were performed on the fully developed leaf 5 at V5 developmental stage using LI-6800 (LI-COR Biosciences GmbH, USA) in a greenhouse experiment and iWUE was calculated as the ratio between CO₂ assimilation and stomatal conductance. Data are means±standard error (n=10). Significant differences between RP and each of the NILs based on Dunnet's test are indicated with dark grey color of the bars (light grey bars do not differ significantly from RP). The black square frame indicates the target genomic region associated with the trait. Coordinates indicated in the last row are according to B73 v4 (maizegdb.org).

FIG. 16. Comparison of the near isogenic line B (NIL B) and nine recombinant NILs (D-L) to their recurrent parent (RP) in terms of stomatal conductance (gs). Each NIL carries an introgression (marked with dark grey) from a flint donor parent in the genetic background of the dent RP (light grey). Leaf gas-exhchange measurements were performed on the fully developed leaf 5 at V5 developmental stage using LI-6800 (LI-COR Biosciences GmbH, USA) to determine gₛ in a greenhouse experiment. Data are means±standard error (n=10). Significant differences between RP and each of the NILs based on Dunnet's test are indicated with dark grey color of the bars (light grey bars do not differ significantly from RP). The black square frame indicates the target genomic region associated with the trait. Coordinates indicated in the last row are according to B73 v4 (maizegdb.org).

FIG. 17. Comparison of the near isogenic line B (NIL B) and nine recombinant NILs (D-L) to their recurrent parent (RP) in terms of stomatal density. Each NIL carries an introgression (marked with dark grey) from a flint donor parent in the genetic background of the dent RP (light grey). Stomata were counted in epidermal imprints taken from on the fully developed leaf 5 at V5 developmental stage in a greenhouse experiment. Data are means±standard error (n=10). Significant differences between RP and each of the NILs based on Dunnet's test are indicated with dark grey color of the bars (light grey bars do not differ significantly from RP). The black square frame indicates the target genomic region associated with the trait. Coordinates indicated in the last row are according to B73 v4 (maizegdb.org).

FIG. 18. Comparison of the near isogenic line B (NIL B) and nine recombinant NILs (D-L) to their recurrent parent (RP) in terms of leaf abscisic acid (ABA) concentrations. Each NIL carries an introgression (marked with dark grey) from a flint donor parent in the genetic background of the dent RP (light grey). ABA concentrations were determined in samples harvested from the fully developed leaf 5 at V5 developmental stage in a greenhouse experiment. Data are means±standard error (n=10). Significant differences between RP and each of the NILs based on Dunnet's test are indicated with dark grey color of the bars (light grey bars do not differ significantly from RP). The black square frame indicates the target genomic region associated with the trait. Coordinates indicated in the last row are according to B73 v4 (maizegdb.org).

FIG. 19. Comparison of the near isogenic line B (NIL B) and nine recombinant NILs (D-L) to their recurrent parent (RP) in terms of leaf phaseic acid (PA) concentrations. Each NIL carries an introgression (marked with dark grey) from a flint donor parent in the genetic background of the dent RP (light grey). PA concentrations were determined in samples harvested from the fully developed leaf 5 at V5 developmental stage in a greenhouse experiment. Data are means±standard error (n=10). Significant differences between RP and each of the NILs based on Dunnet's test are indicated with dark grey color of the bars (light grey bars do not differ significantly from RP). The black square frame indicates the target genomic region associated with the trait. Coordinates indicated in the last row are according to B73 v4 (maizegdb.org).

FIG. 20. Comparison of the near isogenic line B (NIL B) and nine recombinant NILs (D-L) to their recurrent parent (RP) in terms of the ratio of catabolic products phaseic acid (PA) and dihydrophaseic acid (DPA) to their substrate abscisic acid (ABA). Each NIL carries an introgression (marked with dark grey) from a flint donor parent in the genetic background of the dent RP (light grey). Metabolite concentrations were determined in samples harvested from the fully developed leaf 5 at V5 developmental stage in a greenhouse experiment. Data are means±standard error (n=10). Significant differences between RP and each of the NILs based on Dunnet's test are indicated with dark grey color of the bars (light grey bars do not differ significantly from RP). The black square frame indicates the target genomic region associated with the trait. Coordinates indicated in the last row are according to B73 v4 (maizegdb.org).

FIG. 21. Comparison of the near isogenic line B (NIL B) and nine recombinant NILs (D-L) to their recurrent parent (RP) in terms kernel carbon isotope composition (δ¹³C). Each NIL carries an introgression (marked with dark grey) from a flint donor parent in the genetic background of the dent RP (light grey). δ¹³C was determined in kernels harvested in a greenhouse experiment. Data are means±standard error (n=10). Significant differences between RP and each of the NILs based on Dunnet's test are indicated with dark grey color of the bars (light grey bars do not differ significantly from RP). The black square frame indicates the target genomic region associated with the trait. Coordinates indicated in the last row are according to B73 v4 (maizegdb.org).

FIG. 22. Comparison of the near isogenic line B (NIL B) and nine recombinant NILs (D-L) to their recurrent parent (RP) in terms kernel carbon isotope composition (δ¹³C). Each NIL carries an introgression (marked with dark grey) from a flint donor parent in the genetic background of the dent RP (light grey). δ¹³C was determined in kernels harvested in a field experiment in well-watered conditions. Data are means±standard error (n=10). Significant differences between RP and each of the NILs based on Dunnet's test are indicated with dark grey color of the bars (light grey bars do not differ significantly from RP). The black square frame indicates the target genomic region associated with the trait. Coordinates indicated in the last row are according to B73 v4 (maizegdb.org).

FIG. 23. Comparison of the near isogenic line B (NIL B) and nine recombinant NILs (D-L) to their recurrent parent (RP) in terms kernel carbon isotope composition (δ¹³C). Each NIL carries an introgression (marked with dark grey) from a flint donor parent in the genetic background of the dent RP (light grey). δ¹³C was determined in kernels harvested in a rain-out shelter under mild drought conditions. Data are means±standard error (n=10). Significant differences between RP and each of the NILs based on Dunnet's test are indicated with dark grey color of the bars (light grey bars do not differ significantly from RP). The black square frame indicates the target genomic region associated with the trait. Coordinates indicated in the last row are according to B73 v4 (maizegdb.org).

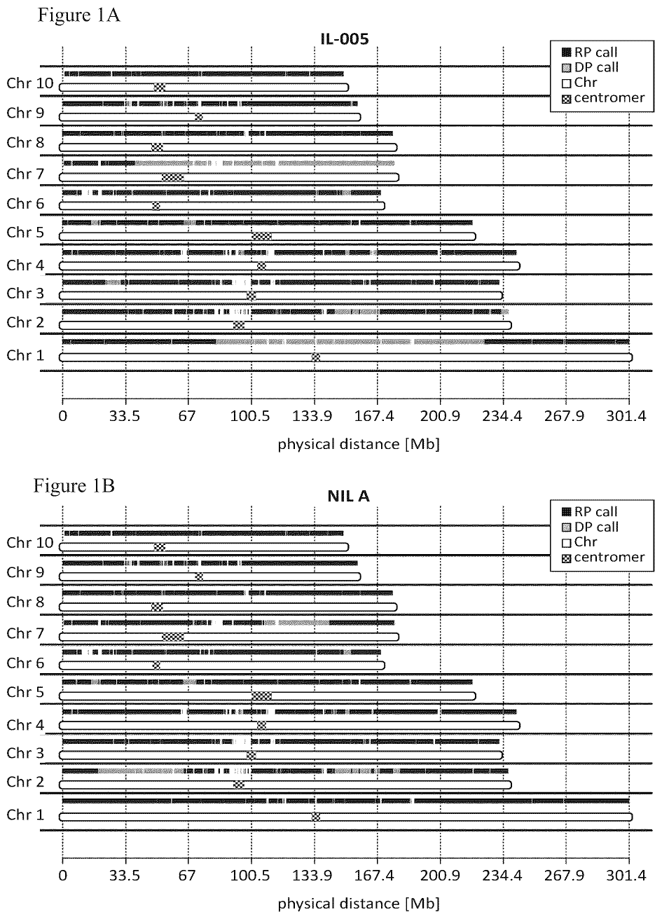

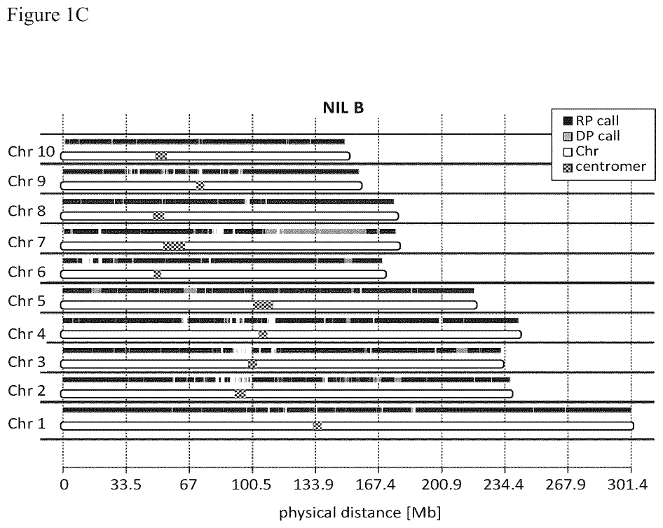

Sequences

| SEQ ID NO: | description |
|---|---|
| 1 | genomic DNA of ZmCSLE1 derived from B73 |
| 2 | cDNA of ZmCSLE1 derived from B73 |
| 3 | amino acid sequences of ZmCSLE1 derived from B73 |
| 4 | genomic DNA of ZmCSLE1 derived from PH207 |
| 5 | cDNA of ZmCSLE1 derived from PH207 |
| 6 | amino acid sequences of ZmCSLE1 derived from PH207 |
| 7 | genomic DNA of ZmCSLE1 derived from B73 including |
| | upstream and downstream flanking regions |
| 8 | genomic DNA of ZmCSLE1 derived from PH207 including |
| | upstream and downstream flanking regions |
| 9 | genomic DNA of ZmAbh4 derived from B73 |
| 10 | transcript 1 of ZmAbh4 derived from B73 |
| 11 | cDNA of transcript 1 of ZmAbh4 derived from B73 |
| 12 | amino acid sequences of transcript 1 of ZmAbh4 |
| | derived from B73 |
| 13 | transcript 2 of ZmAbh4 derived from B73 |
| 14 | cDNA of transcript 2 of ZmAbh4 derived from B73 |
| 15 | amino acid sequences of transcript 2 and 3 of |
| | ZmAbh4 derived from B73 |
| 16 | transcript 3 of ZmAbh4 derived from B73 |
| 17 | cDNA of transcript 3 of ZmAbh4 derived from B73 |
| 18 | genomic DNA of ZmAbh4 derived from PH207 |
| 19 | transcript of ZmAbh4 derived from PH207 |
| 20 | cDNA of ZmAbh4 derived from PH207 |
| 21 | amino acid sequences of ZmAbh4 derived from PH207 |
| 22 | genomic DNA of ZmAbh4 derived from B73 including |
| | upstream and downstream flanking regions |
| 23 | genomic DNA of ZmAbh4 derived from PH207 including |
| | upstream and downstream flanking regions |
| 24 | genomic DNA of ZmWEB1 derived from B73 |
| 25 | cDNA of ZmWEB1 derived from B73 |
| 26 | amino acid sequences of ZmWEB1 derived from B73 |
| 27 | genomic DNA of ZmWEB1 derived from PH207 |
| 28 | cDNA of ZmWEB1 derived from PH207 |
| 29 | amino acid sequences of ZmWEB1 derived from PH207 |
| 30 | genomic DNA of ZmWEB1 derived from B73 including |
| | upstream and downstream flanking regions |
| 31 | genomic DNA of ZmWEB1 derived from PH207 including |
| | upstream and downstream flanking regions |
| 32 | genomic DNA of GRMZM2G397260 derived from B73 |
| 33 | cDNA of GRMZM2G397260 derived from B73 |
| 34 | amino acid sequences of GRMZM2G397260 derived from |
| | B73 |
| 35 | genomic DNA of GRMZM2G397260 derived from B73 |
| | including upstream and downstream flanking regions |
| 36 | genomic DNA of ZmHsftf21 derived from B73 |
| 37 | cDNA of ZmHsftf21 derived from B73 |
| 38 | amino acid sequences of ZmHsftf21 derived from B73 |
| 39 | genomic DNA of ZmHsftf21 derived from PH207 |
| 40 | cDNA of ZmHsftf21 derived from PH207 |
| 41 | amino acid sequences of ZmHsftf21 derived from PH207 |
| 42 | genomic DNA of ZmHsftf21 derived from B73 including |
| | upstream and downstream flanking regions |
| 43 | genomic DNA of ZmHsftf21 derived from PH207 including |
| | upstream and downstream flanking regions |

### DETAILED DESCRIPTION OF THE INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous.

As used herein, “maize” refers to a plant of the species Zea mays, preferably Zea mays ssp mays.

The term “plant” includes whole plants, including descendants or progeny thereof. The term “plant part” includes any part or derivative of the plant, including particular plant tissues or structures, plant cells, plant protoplast, plant cell or tissue culture from which plants can be regenerated, plant calli, plant clumps and plant cells that are intact in plants or parts of plants, such as seeds, kernels, cobs, flowers, cotyledons, leaves, stems, buds, roots, root tips, stover, and the like. Plant parts may include processed plant parts or derivatives, including flower, oils, extracts etc.

In certain embodiments, the plant part or derivative comprises, consists of, or consists essentially of one or more, preferably all of stalks, leaves, and cobs. In certain embodiments, the plant part or derivative is leaves. In certain embodiments, the plant part or derivative is stalks. In certain embodiments, the plant part or derivative is cobs. In certain embodiments, the plant part or derivative comprises, consists of, or consists essentially of one or more, preferably all of stalks and leaves. In certain embodiments, the plant part or derivative comprises, consists of, or consists essentially of one or more, preferably all of stalks, and cobs. In certain embodiments, the plant part or derivative comprises, consists of, or consists essentially of one or more, preferably all of leaves and cobs. In certain embodiments, the plant part or derivative is not (functional) propagation material, such as germplasm, a seed, or plant embryo or other material from which a plant can be regenerated. In certain embodiments, the plant part or derivative does not comprise (functional) male and female reproductive organs. In certain embodiments, the plant part or derivative is or comprises propagation material, but propagation material which does not or cannot be used (anymore) to produce or generate new plants, such as propagation material which have been chemically, mechanically or otherwise rendered non-functional, for instance by heat treatment, acid treatment, compaction, crushing, chopping, etc. in certain preferred embodiments, the plant part is corn cobs or stover.

Drought resistance or drought tolerance as referred to herein, relates to is the ability to which a plant maintains its biomass production during arid or drought conditions, i.e. during conditions of suboptimal water supply or availability. The mechanisms behind drought tolerance are complex and involve many pathways which allow plants to respond to specific sets of conditions at any given time. Some of these interactions include stomatal conductance, carotenoid degradation and anthocyanin accumulation, the intervention of osmoprotectants (such as sucrose, glycine, and proline), ROS-scavenging enzymes. The molecular control of drought tolerance is also very complex and is influenced other factors such as environment and the developmental stage of the plant. This control consists mainly of transcriptional factors, such as dehydration-responsive element-binding protein (DREB), abscisic acid (ABA)-responsive element-binding factor (AREB), and NAM (no apical meristem). A drought-resistant or drought-tolerant plant, plant cell or plant part refers herein to a plant, plant cell or plant part, respectively, having increased resistance/tolerance to drought compared to a parent plant from which they are derived. Methods of determining drought resistance/tolerance are known to the person of skill in the art. In certain embodiments, the plants or plant parts are more resistant or more tolerant to drought. In certain embodiments, the plants or plant parts are less resistant or less tolerant to drought. In certain embodiments, the plants or plant parts are more sensitive to drought. In certain embodiments, the plants or plant parts are less sensitive to drought. Less sensitive when used herein may, vice versa, be seen as “more tolerable” or “more resistant”. Similarly, “more tolerable” or “more resistant” may, vice versa, be seen as “less sensitive”. More sensitive when used herein may, vice versa, be seen as “less tolerable” or “less resistant”. Similarly, “less tolerable” or “less resistant” may, vice versa, be seen as “more sensitive”. In certain embodiments, the more drought resistant or tolerant plants exhibit a loss in biomass production (such as expressed in g/day or kg/ha or kg/ha/day, such as expressed as dry matter for instance expressed as weight percent) under drought conditions which is at least 1%, preferably at least 2%, such as at least 3%, at least 4%, at least 5%, or more lower than corresponding control plants, such as plants which are less drought resistant or tolerant, or plants not comprising the QTL (allele) or markers or marker alleles according to the invention as described herein.

δ13C as used herein refers to an isotopic signature, a measure of the ratio of stable isotopes 13C:12C (i.e. carbon isotope composition), reported in parts per thousand (per mil, % o). δ13C is calculated as follows:

$$δ13 C = ( \frac{( \frac{13 C}{12 C} ) sample}{( \frac{13 C}{12 C} ) standard} - 1 ) \times 1000$$

where the standard is the established reference material. The standard established for carbon-13 work was the Pee Dee Belemnite (PDB) and was based on a Cretaceous marine fossil, Belemnitella americana, which was from the Peedee Formation in South Carolina. This material had an anomalously high 13C:12C ratio (0.01118), and was established as δ13C value of zero. Since the original PDB specimen is no longer available, its 13C:12C ratio is currently back-calculated from a widely measured carbonate standard NBS-19, which has a δ13C value of +1.95‰.[3] The 13C:12C ratio of NBS-19 is 0.011078/0.988922=0.011202. Therefore the correct 13C:12C ratio of PDB derived from NBS-19 should be 0,011202/(1,95/1000+1)=0,011202/1,00195=0,01118.

δ13C varies in time as a function of productivity, the signature of the inorganic source, organic carbon burial and vegetation type. Biological processes preferentially take up the lower mass isotope through kinetic fractionation. However some abiotic processes do the same, methane from hydrothermal vents can be depleted by up to 50%.

Carbon in materials originated by photosynthesis is depleted of the heavier isotopes. In addition, there are two types of plants with different biochemical pathways; the C3 carbon fixation, where the isotope separation effect is more pronounced, C4 carbon fixation, where the heavier 13C is less depleted, and Crassulacean Acid Metabolism (CAM) plants, where the effect is similar but less pronounced than with C4 plants. Isotopic fractionation in plants is caused by physical (slower diffusion of 13C in plant tissues due to increased atomic weight) and biochemical (preference of 12C by two enzymes: RuBisCO and phosphoenolpyruvate carboxylase) factors.

Carbon isotope composition can be used as proxy for inferring information about transpiration efficiency in C3 species (Farquhar et al., 1989. Carbon isotope discrimination and photosynthesis. Annual review of plant biology, 40(1), 503-537). Several studies in C4 species have shown negative correlations between δ13C and water use efficiency (WUE; Henderson et al., 1998. Correlation between carbon isotope discrimination and transpiration efficiency in lines of the C4 species Sorghum bicolor in the glasshouse and the field. Functional Plant Biology, 25(1), 111-123; Dercon et al., 2006. Differential 13 C isotopic discrimination in maize at varying water stress and at low to high nitrogen availability. Plant and Soil, 282(1-2), 313-326; Sharwood et al., 2014. Photosynthetic flexibility in maize exposed to salinity and shade. Journal of experimental botany, 65(13), 3715-3724.), which is defined as the amount of biomass or yield accumulated per unit of water used.

In the context of the present invention, a particular QTL or marker is said to be “associated with” or “affects” a particular trait or parameter, such as drought resistance/tolerance or δ13C, if the trait or parameter value varies (i.e. exhibits a phenotypical difference) depending on the identity of the QTL or marker (i.e. the sequence). Such correlation may be causative or non-causative.

As used herein, the term “stomatal parameter” refers to any parameter related to, influencing, or resulting from stomata functionality, structure (including size, distribution, density), etc. As used herein, the term “gas exchange parameter” refers to any parameter related to, influencing, or resulting from uptake and/or release of gasses (such as CO₂, O₂, H₂O) to and from the plant. The skilled person will understand that to some extent stomatal and gas exchange parameters may be interlinked or overlapping.

As used herein, the term water use efficiency (WUE) refers to the ratio between effective water use and actual water withdrawal. It characterizes, in a specific process, how effective is the use of water. WUE can be expressed as the ratio of water used in plant metabolism to water lost by the plant through transpiration. WUE can be measured at different scales, ranging from instantaneous measurements on the leaf to more integrative ones at the plant and crop levels. Intrinsic water use efficiency (iWUE) is the ratio of net CO₂ assimilation rate to stomatal conductance (A/gₛ; expressed in mol CO₂/mol H₂O). Whole plant water use efficiency (WUE plant) is the ratio of the difference between final and initial plant biomass and the total amount of water consumed (expressed in g/I). Lifetime-integrated proxies of WUE are measured as the ratio of 13C to 12C (Δ13C or δ13C).

As used herein, the term stomatal conductance (gₛ; expressed in mol/m²/s) refers to rate of passage of carbon dioxide (CO₂) entering, or water vapour exiting through the stomata of a leaf. Stomatal conductance is a function of stomatal density, stomatal aperture, and stomatal size. Stomatal conductance can be measured by means known in the art, such as steady-state porometers, dynamic porometers, or null balance porometers.

As used herein, the term net CO₂ assimilation rate (A; expressed in mol/m²/s) refers to the photosynthetic assimilation of CO₂ per leaf area over a given time frame. Net CO₂ assimilation rate can be measured by means known in the art.

As used herein, the term transpiration (E; expressed in ml/g or ml/m² or ml/g/s or ml/m²/s for transpiration rate) refers to the process of water movement through a plant and its evaporation from aerial parts, such as leaves, stems and flowers. Transpiration occurs through the stomatal apertures. Transpiration can be measured by means known in the art.

As used herein, the term stomatal density refers to the amount of stomata per leaf area.

As used herein, the term ABA content refers to the amount or concentration of abscisic acid. ABA content can for instance be determined as ABA content in various plant tissues or organs, such as ABA leaf content.

As used herein, the term sensitivity of growth to drought refers to the influence of drought or water availability in general on growth characteristics (such as for instance biomass production). An increased sensitivity of growth to drought is reflected by a higher (negative) impact of drought on growth.

As used herein, the B73 reference genome AGPv2 refers to the assembly B73 RefGen_v2 (also known as AGPv2, B73 RefGen_v2) as provided on the Maize Genetics and Genomics Database (maizegdb.org/genome/genome_assembly/B73%20RefGen_v2) and in US20150322452.

As used herein, the B73 reference genome AGPv4 refers to the assembly B73 RefGen_v[[2]]4 (also known as AGPv4, B73 RefGen_v4) as provided on the Maize Genetics and Genomics Database (maizegdb.org/genome/genome_assembly/Zm-B73-REFERENCE-GRAMENE-4.0).

As referred to herein, a polynucleic acid, such as for instance a QTL (allele) as described herein, is said to be flanked by certain molecular markers or molecular marker alleles if the polynucleic acid is comprised within a polynucleic acid wherein respectively a first marker (allele) is located upstream (i.e. 5′) of said polynucleic acid and a second marker (allele) is located downstream (i.e. 3′) of said polynucleic acid. Such first and second marker (allele) may border the polynucleic acid. The nucleic acid may equally comprise such first and second marker (allele), such as respectively at or near the 5′ and 3′ end, for instance respectively within 50 kb of the 5′ and 3′ end, preferably within 10 kb of the 5′ and 3′ end, such as within 5 kb of the 5′ and 3′ end, within 1 kb of the 5′ and 3′ end, or less.

As used herein, increased (protein and/or mRNA) expression levels refers to increased expression levels of about at least 10%, preferably at least 30%, more preferably at least 50%, such as at least 20%, 40%, 60%, 80% or more, such as at least 85%, at least 90%, at least 95%, or more. As used herein, reduced (protein and/or mRNA) expression levels refers to decreased expression levels of about at least 10%, preferably at least 30%, more preferably at least 50%, such as at least 20%, 40%, 60%, 80% or more, such as at least 85%, at least 90%, at least 95%, or more. Expression is (substantially) absent or eliminated if expression levels are reduced at least 80%, preferably at least 90%, more preferably at least 95%. In certain embodiments, expression is (substantially) absent, if no protein and/or mRNA, in particular the wild type or native protein and/or mRNA, can be detected. Expression levels can be determined by any means known in the art, such as by standard detection methods, including for instance (quantitative) PCR, northern blot, western blot, ELISA, etc.

As used herein, increased (protein) activity refers to increased activity of about at least 10%, preferably at least 30%, more preferably at least 50%, such as at least 20%, 40%, 60%, 80% or more, such as at least 85%, at least 90%, at least 95%, or more. As used herein, reduced (protein) activity refers to decreased activity of about at least 10%, preferably at least 30%, more preferably at least 50%, such as at least 20%, 40%, 60%, 80% or more, such as at least 85%, at least 90%, at least 95%, or more. Activity is (substantially) absent or eliminated if activity is reduced at least 80%, preferably at least 90%, more preferably at least 95%. In certain embodiments, activity is (substantially) absent, if no activity, in particular the wild type or native protein activity, can be detected. (Protein) activity levels can be determined by any means known in the art, depending on the type of protein, such as by standard detection methods, including for instance enzymatic assays (for enzymes), transcription assays (for transcription factors), assays to analyse a phenotypic output, etc.

Expression levels or activity may be compared between different plants (or plant parts), such as a plant (part) comprising the QTL (allele) and/or marker(s) (allele(s)) according to the invention and a plant (part) not comprising the QTL (allele) and/or marker(s) (allele(s)) according to the invention. Expression levels or activity may be compared between different conditions, such as drought conditions and non-drought conditions. Expression levels or activity may be compared with a predetermined threshold. Such predetermined threshold may for instance correspond to expression levels or activity in a particular genotype (for instance in a plant not comprising the QTL (allele) and/or marker(s) (allele(s)) according to the invention) or under particular conditions (such as for instance under non-drought conditions).

The term “locus” (loci plural) means a specific place or places or a site on a chromosome where for example a QTL, a gene or genetic marker is found. As used herein, the term “quantitative trait locus” or “QTL” has its ordinary meaning known in the art. By means of further guidance, and without limitation, a QTL may refer to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window. A QTL may encode for one or more alleles that affect the expressivity of a continuously distributed (quantitative) phenotype. In certain embodiments, the QTL as described herein may be homozygous. In certain embodiments, the QTL as described herein may be heterozygous.

As used herein, the term “allele” or “alleles” refers to one or more alternative forms, i.e. different nucleotide sequences, of a locus.

As used herein, the term “mutant alleles” or “mutation” of alleles include alleles having one or more mutations, such as insertions, deletions, stop codons, base changes (e.g., transitions or transversions), or alterations in splice junctions, which may or may not give rise to altered gene products. Modifications in alleles may arise in coding or non-coding regions (e.g. promoter regions, exons, introns or splice junctions).

As used herein, the terms “introgression”, “introgressed” and “introgressing” refer to both a natural and artificial process whereby chromosomal fragments or genes of one species, variety or cultivar are moved into the genome of another species, variety or cultivar, by crossing those species. The process may optionally be completed by backcrossing to the recurrent parent. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line having a desired genetic background and selected for the desired allele, to result in the allele becoming fixed in a selected genetic background. The process of “introgressing” is often referred to as “backcrossing” when the process is repeated two or more times. “Introgression fragment” or “introgression segment” or “introgression region” refers to a chromosome fragment (or chromosome part or region) which has been introduced into another plant of the same or related species either artificially or naturally such as by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even three quarter or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less. In certain embodiments, the introgression fragment comprises, consists of, or consists essentially of the QTL according to the invention as described herein.

A genetic element, an introgression fragment, or a gene or allele conferring a trait (such as improved digestibility) is said to be “obtainable from” or can be “obtained from” or “derivable from” or can be “derived from” or “as present in” or “as found in” a plant or plant part as described herein elsewhere if it can be transferred from the plant in which it is present into another plant in which it is not present (such as a line or variety) using traditional breeding techniques without resulting in a phenotypic change of the recipient plant apart from the addition of the trait conferred by the genetic element, locus, introgression fragment, gene or allele. The terms are used interchangeably and the genetic element, locus, introgression fragment, gene or allele can thus be transferred into any other genetic background lacking the trait. Not only pants comprising the genetic element, locus, introgression fragment, gene or allele can be used, but also progeny/descendants from such plants which have been selected to retain the genetic element, locus, introgression fragment, gene or allele, can be used and are encompassed herein. Whether a plant (or genomic DNA, cell or tissue of a plant) comprises the same genetic element, locus, introgression fragment, gene or allele as obtainable from such plant can be determined by the skilled person using one or more techniques known in the art, such as phenotypic assays, whole genome sequencing, molecular marker analysis, trait mapping, chromosome painting, allelism tests and the like, or combinations of techniques. It will be understood that transgenic plants may also be encompassed.

As used herein the terms “genetic engineering”, “transformation” and “genetic modification” are all used herein as synonyms for the transfer of isolated and cloned genes into the DNA, usually the chromosomal DNA or genome, of another organism.

“Transgenic” or “genetically modified organisms” (GMOs) as used herein are organisms whose genetic material has been altered using techniques generally known as “recombinant DNA technology”. Recombinant DNA technology encompasses the ability to combine DNA molecules from different sources into one molecule ex vivo (e.g. in a test tube). This terminology generally does not cover organisms whose genetic composition has been altered by conventional cross-breeding or by “mutagenesis” breeding, as these methods predate the discovery of recombinant DNA techniques. “Non-transgenic” as used herein refers to plants and food products derived from plants that are not “transgenic” or “genetically modified organisms” as defined above.

“Transgene” or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

“Gene editing” or “genome editing” refers to genetic engineering in which in which DNA or RNA is inserted, deleted, modified or replaced in the genome of a living organism. Gene editing may comprise targeted or non-targeted (random) mutagenesis. Targeted mutagenesis may be accomplished for instance with designer nucleases, such as for instance with meganucleases, zinc finger nucleases (ZFNs), transcription activator-like effector-based nucleases (TALEN), and the clustered regularly interspaced short palindromic repeats (CRISPR/Cas9) system. These nucleases create site-specific double-strand breaks (DSBs) at desired locations in the genome. The induced double-strand breaks are repaired through nonhomologous end-joining (NHEJ) or homologous recombination (HR), resulting in targeted mutations or nucleic acid modifications. The use of designer nucleases is particularly suitable for generating gene knockouts or knockdowns. In certain embodiments, designer nucleases are developed which specifically induce a mutation in the F35H gene, as described herein elsewhere, such as to generate a mutated F35H or a knockout of the F35H gene. In certain embodiments, designer nucleases, in particular RNA-specific CRISPR/Cas systems are developed which specifically target the F35H mRNA, such as to cleave the F35H mRNA and generate a knockdown of the F35H gene/mRNA/protein. Delivery and expression systems of designer nuclease systems are well known in the art.

In certain embodiments, the nuclease or targeted/site-specific/homing nuclease is, comprises, consists essentially of, or consists of a (modified) CRISPR/Cas system or complex, a (modified) Cas protein, a (modified) zinc finger, a (modified) zinc finger nuclease (ZFN), a (modified) transcription factor-like effector (TALE), a (modified) transcription factor-like effector nuclease (TALEN), or a (modified) meganuclease. In certain embodiments, said (modified) nuclease or targeted/site-specific/homing nuclease is, comprises, consists essentially of, or consists of a (modified) RNA-guided nuclease. It will be understood that in certain embodiments, the nucleases may be codon optimized for expression in plants. As used herein, the term “targeting” of a selected nucleic acid sequence means that a nuclease or nuclease complex is acting in a nucleotide sequence specific manner. For instance, in the context of the CRISPR/Cas system, the guide RNA is capable of hybridizing with a selected nucleic acid sequence. As uses herein, “hybridization” or “hybridizing” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PGR, or the cleavage of a polynucleotide by an enzyme. A sequence capable of hybridizing with a given sequence is referred to as the “complement” of the given sequence.

Gene editing may involve transient, inducible, or constitutive expression of the gene editing components or systems. Gene editing may involve genomic integration or episomal presence of the gene editing components or systems. Gene editing components or systems may be provided on vectors, such as plasmids, which may be delivered by appropriate delivery vehicles, as is known in the art. Preferred vectors are expression vectors.

Gene editing may comprise the provision of recombination templates, to effect homology directed repair (HDR). For instance a genetic element may be replaced by gene editing in which a recombination template is provided. The DNA may be cut upstream and downstream of a sequence which needs to be replaced. As such, the sequence to be replaced is excised from the DNA. Through HDR, the excised sequence is then replaced by the template. In certain embodiments, the QTL allele of the invention as described herein may be provided on/as a template. By designing the system such that double strand breaks are introduced upstream and downstream of the corresponding region in the genome of a plant not comprising the QTL allele, this region is excised and can be replaced with the template comprising the QTL allele of the invention. In this way, introduction of the QTL allele of the invention in a plant need not involve multiple backcrossing, in particular in a plant of specific genetic background. Similarly, the mutated F35H of the invention may be provided on/as a template. More advantageously however, the mutated F35H of the invention may be generated without the use of a recombination template, but solely through the endonuclease action leading to a double strand DNA break which is repaired by NHEJ, resulting in the generation of indels.

In certain embodiments, the nucleic acid modification or mutation is effected by a (modified) transcription activator-like effector nuclease (TALEN) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference. By means of further guidance, and without limitation, naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26. The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference. By means of further guidance, and without limitation, artificial zinc-finger (ZF) technology involves arrays of ZF modules to target new DNA-binding sites in the genome. Each finger module in a ZF array targets three DNA bases. A customized array of individual zinc finger domains is assembled into a ZF protein (ZFP). ZFPs can comprise a functional domain. The first synthetic zinc finger nucleases (ZFNs) were developed by fusing a ZF protein to the catalytic domain of the Type IlS restriction enzyme Fokl. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. With respect to general information on CRISPR/Cas Systems, components thereof, and delivery of such components, including methods, materials, delivery vehicles, vectors, particles, and making and using thereof, including as to amounts and formulations, as well as Cas9CRISPR/Cas-expressing eukaryotic cells, Cas-9 CRISPR/Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), WO 2015/089351 (PCT/US2014/069897), WO 2015/089354 (PCT/US2014/069902), WO 2015/089364 (PCT/US2014/069925), WO 2015/089427 (PCT/US2014/070068), WO 2015/089462 (PCT/US2014/070127), WO 2015/089419 (PCT/US2014/070057), WO 2015/089465 (PCT/US2014/070135), WO 2015/089486 (PCT/US2014/070175), PCT/US2015/051691, PCT/US2015/051830. Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/835,973, 61/836,080, 61/836,101, and 61/836,127, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT/US2014/62558 filed Oct. 28, 2014, and U.S. Provisional Patent Applications Ser. Nos. 61/915,148, 61/915,150, 61/915,153, 61/915,203, 61/915,251, 61/915,301, 61/915,267, 61/915,260, and 61/915,397, each filed Dec. 12, 2013; 61/757,972 and 61/768,959, filed on Jan. 29, 2013 and Feb. 25, 2013; 62/010,888 and 62/010,879, both filed Jun. 11, 2014; 62/010,329, 62/010,439 and 62/010,441, each filed Jun. 10, 2014; 61/939,228 and 61/939,242, each filed Feb. 12, 2014; 61/980,012, filed Apr. 15,2014; 62/038,358, filed Aug. 17, 2014; 62/055,484, 62/055,460 and 62/055,487, each filed Sep. 25, 2014; and 62/069,243, filed Oct. 27, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to PCT application designating, inter alia, the United States, application No. PCT/US14/41806, filed Jun. 10, 2014. Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS. Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, and Attorney Docket No. 46783.01.2128, filed 22 Oct. 2015, NOVEL CRISPR ENZYMES AND SYSTEMS, U.S. application 62/232,067, 24 Sep. 2015, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES. European patent application EP3009511. Reference is further made to Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013); RNA-guided editing of bacterial genomes using CRISPR-Cas systems. Jiang W., Bikard D., Cox D., Zhang F, Marraffini L A. Nat Biotechnol March; 31(3):233-9 (2013); One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013); Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. 2013 Aug. 22; 500(7463):472-6. doi: 10.1038/Nature12466. Epub 2013 Aug. 23; Double Nicking by RNA-Guided CRISPR Cas9 for Enhanced Genome Editing Specificity. Ran, FA., Hsu, PD., Lin, CY., Gootenberg, J S., Konermann, S., Trevino, AE., Scott, DA., Inoue, A., Matoba, S., Zhang, Y., & Zhang, F. Cell August 28. pii: S0092-8674(13)01015-5. (2013); DNA targeting specificity of RNA-guided Cas9 nucleases. Hsu, P., Scott, D., Weinstein, J., Ran, FA., Konermann, S., Agarwala, V., Li, Y., Fine, E., Wu, X., Shalem, O., Cradick, TJ., Marraffini, LA., Bao, G., & Zhang, F. Nat Biotechnol doi:10.1038/nbt.2647 (2013); Genome engineering using the CRISPR-Cas9 system. Ran, FA., Hsu, PD., Wright, J., Agarwala, V., Scott, DA., Zhang, F. Nature Protocols November; 8(11):2281-308. (2013); Genome-Scale CRISPR-Cas9 Knockout Screening in Human Cells. Shalem, O., Sanjana, NE., Hartenian, E., Shi, X., Scott, DA., Mikkelson, T., Heckl, D., Ebert, BL., Root, DE., Doench, JG., Zhang, F. Science December 12. (2013). [Epub ahead of print]; Crystal structure of cas9 in complex with guide RNA and target DNA. Nishimasu, H., Ran, FA., Hsu, PD., Konermann, S., Shehata, SI., Dohmae, N., Ishitani, R., Zhang, F., Nureki, O. Cell February 27. (2014). 156(5):935-49; Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. (2014) April 20. doi: 10.1038/nbt.2889; CRISPR-Cas9 Knockin Mice for Genome Editing and Cancer Modeling, Platt et al., Cell 159(2): 440-455 (2014) DOI: 10.1016/j.cell.2014.09.014; Development and Applications of CRISPR-Cas9 for Genome Engineering, Hsu et al, Cell 157, 1262-1278 (Jun. 5, 2014) (Hsu 2014); Genetic screens in human cells using the CRISPR/Cas9 system, Wang et al., Science. 2014 January 3; 343(6166): 80-84. doi:10.1126/science.1246981; Rational design of highly active sgRNAs for CRISPR-Cas9-mediated gene inactivation, Doench et al., Nature Biotechnology 32(12):1262-7 (2014) published online 3 Sep. 2014; doi:10.1038/nbt.3026, and In vivo interrogation of gene function in the mammalian brain using CRISPR-Cas9, Swiech et al, Nature Biotechnology 33, 102-106 (2015) published online 19 Oct. 2014; doi:10.1038/nbt.3055, Cpf1 Is a Single RNA-Guided Endonuclease of a Class 2 CRISPR-Cas System, Zetsche et al., Cell 163, 1-13 (2015); Discovery and Functional Characterization of Diverse Class 2 CRISPR-Cas Systems, Shmakov et al., Mol Cell 60(3): 385-397 (2015); C2c2 is a single-component programmable RNA-guided RNA-targeting CRISPR effector, Abudayyeh et al, Science (2016) published online Jun. 2, 2016 doi: 10.1126/science.aaf5573. Each of these publications, patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cpf1).

As used herein, the term “crRNA” or “guide RNA” or “single guide RNA” or “sgRNA” or “one or more nucleic acid components” of a CRISPR/Cas locus effector protein, as applicable, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay.

A guide sequence, and hence a nucleic acid-targeting guide RNA may be selected to target any target nucleic acid sequence. The target sequence may be DNA. The target sequence may be genomic DNA. The target sequence may be mitochondrial DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In certain embodiments, the gRNA comprises a stem loop, preferably a single stem loop. In certain embodiments, the direct repeat sequence forms a stem loop, preferably a single stem loop. In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In particular embodiments, the CRISPR/Cas system requires a tracrRNA. The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and gRNA sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop may correspond to the tracr mate sequence, and the portion of the sequence 3′ of the loop then corresponds to the tracr sequence. In a hairpin structure the portion of the sequence 5′ of the final “N” and upstream of the loop may alternatively correspond to the tracr sequence, and the portion of the sequence 3′ of the loop corresponds to the tracr mate sequence. In alternative embodiments, the CRISPR/Cas system does not require a tracrRNA, as is known by the skilled person.

In certain embodiments, the guide RNA (capable of guiding Cas to a target locus) may comprise (1) a guide sequence capable of hybridizing to a target locus and (2) a tracr mate or direct repeat sequence (in 5′ to 3′ orientation, or alternatively in 3′ to 5′ orientation, depending on the type of Cas protein, as is known by the skilled person). In particular embodiments, the CRISPR/Cas protein is characterized in that it makes use of a guide RNA comprising a guide sequence capable of hybridizing to a target locus and a direct repeat sequence, and does not require a tracrRNA. In particular embodiments, where the CRISPR/Cas protein is characterized in that it makes use of a tracrRNA, the guide sequence, tracr mate, and tracr sequence may reside in a single RNA, i.e. an sgRNA (arranged in a 5′ to 3′ orientation or alternatively arranged in a 3′ to 5′ orientation), or the tracr RNA may be a different RNA than the RNA containing the guide and tracr mate sequence. In these embodiments, the tracr hybridizes to the tracr mate sequence and directs the CRISPR/Cas complex to the target sequence.

Typically, in the context of an endogenous nucleic acid-targeting system, formation of a nucleic acid-targeting complex (comprising a guide RNA hybridized to a target sequence and complexed with one or more nucleic acid-targeting effector proteins) results in modification (such as cleavage) of one or both DNA or RNA strands in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest). The skilled person will be aware of specific cut sites for selected CRISPR/Cas systems, relative to the target sequence, which as is known in the art may be within the target sequence or alternatively 3′ or 5′ of the target sequence.

In some embodiments, the unmodified nucleic acid-targeting effector protein may have nucleic acid cleavage activity. In some embodiments, the nuclease as described herein may direct cleavage of one or both nucleic acid (DNA, RNA, or hybrids, which may be single or double stranded) strands at the location of or near a target sequence, such as within the target sequence and/or within the complement of the target sequence or at sequences associated with the target sequence. In some embodiments, the nucleic acid-targeting effector protein may direct cleavage of one or both DNA or RNA strands within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt (e.g. for Cas9, such as SaCas9 or SpCas9). In some embodiments, the cleavage may be staggered (e.g. for Cpf1), i.e. generating sticky ends. In some embodiments, the cleavage is a staggered cut with a 5′ overhang. In some embodiments, the cleavage is a staggered cut with a 5′ overhang of 1 to 5 nucleotides, preferably of 4 or 5 nucleotides. In some embodiments, the cleavage site is upstream of the PAM. In some embodiments, the cleavage site is downstream of the PAM. In some embodiments, the nucleic acid-targeting effector protein that may be mutated with respect to a corresponding wild-type enzyme such that the mutated nucleic acid-targeting effector protein lacks the ability to cleave one or both DNA or RNA strands of a target polynucleotide containing a target sequence. As a further example, two or more catalytic domains of a Cas protein (e.g. RuvC I, RuvC II, and RuvC III or the HNH domain of a Cas9 protein) may be mutated to produce a mutated Cas protein substantially lacking all DNA cleavage activity. In some embodiments, a nucleic acid-targeting effector protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. As used herein, the term “modified” Cas generally refers to a Cas protein having one or more modifications or mutations (including point mutations, truncations, insertions, deletions, chimeras, fusion proteins, etc.) compared to the wild type Cas protein from which it is derived. By derived is meant that the derived enzyme is largely based, in the sense of having a high degree of sequence homology with, a wildtype enzyme, but that it has been mutated (modified) in some way as known in the art or as described herein.

In certain embodiments, the target sequence should be associated with a PAM (protospacer adjacent motif) or PFS (protospacer flanking sequence or site); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). Examples of PAM sequences are given in the examples section below, and the skilled person will be able to identify further PAM sequences for use with a given CRISPR enzyme. Further, engineering of the PAM Interacting (PI) domain may allow programing of PAM specificity, improve target site recognition fidelity, and increase the versatility of the Cas, e.g. Cas9, genome engineering platform. Cas proteins, such as Cas9 proteins may be engineered to alter their PAM specificity, for example as described in Kleinstiver B P et al. Engineered CRISPR-Cas9 nucleases with altered PAM specificities. Nature. 2015 Jul. 23; 523(7561):481-5. doi: 10.1038/nature14592. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence. The skilled person will understand that other Cas proteins may be modified analogously.

The Cas protein as referred to herein, such as without limitation Cas9, Cpf1 (Cas12a), C2c1 (Cas12b), C2c2 (Cas13a), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is (modified) Cpf1, preferably Acidaminococcus sp., such as Acidaminococcus sp. BV3L6 Cpf1 (AsCpf1) or Lachnospiraceae bacterium Cpf1, such as Lachnospiraceae bacterium MA2020 or Lachnospiraceae bacterium MD2006 (LbCpf1). In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b.

In certain embodiments, the nucleic acid modification is effected by random mutagenesis. Cells or organisms may be exposed to mutagens such as UV radiation or mutagenic chemicals (such as for instance such as ethyl methanesulfonate (EMS)), and mutants with desired characteristics are then selected. Mutants can for instance be identified by TILLING (Targeting Induced Local Lesions in Genomes). The method combines mutagenesis, such as mutagenesis using a chemical mutagen such as ethyl methanesulfonate (EMS) with a sensitive DNA screening-technique that identifies single base mutations/point mutations in a target gene. The TILLING method relies on the formation of DNA heteroduplexes that are formed when multiple alleles are amplified by PCR and are then heated and slowly cooled. A “bubble” forms at the mismatch of the two DNA strands, which is then cleaved by a single stranded nucleases. The products are then separated by size, such as by HPLC. See also McCallum et al. “Targeted screening for induced mutations”; Nat Biotechnol. 2000 April; 18(4):455-7 and McCallum et al. “Targeting induced local lesions IN genomes (TILLING) for plant functional genomics”; Plant Physiol. 2000 June; 123(2):439-42.

RNA interference (RNAi) is a biological process in which RNA molecules inhibit gene expression or translation, by neutralizing targeted mRNA molecules. Two types of small ribonucleic acid (RNA) molecules—microRNA (miRNA) and small interfering RNA (siRNA)—are central to RNA interference. RNAs are the direct products of genes, and these small RNAs can bind to other specific messenger RNA (mRNA) molecules and either increase or decrease their activity, for example by preventing an mRNA from being translated into a protein. The RNAi pathway is found in many eukaryotes, including animals, and is initiated by the enzyme Dicer, which cleaves long double-stranded RNA (dsRNA) molecules into short double-stranded fragments of about 21 nucleotide siRNAs (small interfering RNAs). Each siRNA is unwound into two single-stranded RNAs (ssRNAs), the passenger strand and the guide strand. The passenger strand is degraded and the guide strand is incorporated into the RNA-induced silencing complex (RISC). Mature miRNAs are structurally similar to siRNAs produced from exogenous dsRNA, but before reaching maturity, miRNAs must first undergo extensive post-transcriptional modification. A miRNA is expressed from a much longer RNA-coding gene as a primary transcript known as a pri-miRNA which is processed, in the cell nucleus, to a 70-nucleotide stem-loop structure called a pre-miRNA by the microprocessor complex. This complex consists of an RNase Ill enzyme called Drosha and a dsRNA-binding protein DGCR8. The dsRNA portion of this pre-miRNA is bound and cleaved by Dicer to produce the mature miRNA molecule that can be integrated into the RISC complex; thus, miRNA and siRNA share the same downstream cellular machinery. A short hairpin RNA or small hairpin RNA (shRNA/Hairpin Vector) is an artificial RNA molecule with a tight hairpin turn that can be used to silence target gene expression via RNA interference. The most well-studied outcome is post-transcriptional gene silencing, which occurs when the guide strand pairs with a complementary sequence in a messenger RNA molecule and induces cleavage by Argonaute 2 (Ago2), the catalytic component of the RISC. As used herein, an RNAi molecule may be an siRNA, shRNA, or a miRNA. In will be understood that the RNAi molecules can be applied as such to/in the plant, or can be encoded by appropriate vectors, from which the RNAi molecule is expressed. Delivery and expression systems of RNAi molecules, such as siRNAs, shRNAs or miRNAs are well known in the art.

As used herein, the term “homozygote” refers to an individual cell or plant having the same alleles at one or more or all loci. When the term is used with reference to a specific locus or gene, it means at least that locus or gene has the same alleles. As used herein, the term “homozygous” means a genetic condition existing when identical alleles reside at corresponding loci on homologous chromosomes. As used herein, the term “heterozygote” refers to an individual cell or plant having different alleles at one or more or all loci. When the term is used with reference to a specific locus or gene, it means at least that locus or gene has different alleles. As used herein, the term “heterozygous” means a genetic condition existing when different alleles reside at corresponding loci on homologous chromosomes. In certain embodiments, the QTL and/or one or more marker(s) as described herein is/are homozygous. In certain embodiments, the QTL and/or one or more marker(s) as described herein are heterozygous. In certain embodiments, the QTL allele and/or one or more marker(s) allele(s) as described herein is/are homozygous. In certain embodiments, the QTL allele and/or one or more marker(s) allele(s) as described herein are heterozygous.

A “marker” is a (means of finding a position on a) genetic or physical map, or else linkages among markers and trait loci (loci affecting traits). The position that the marker detects may be known via detection of polymorphic alleles and their genetic mapping, or else by hybridization, sequence match or amplification of a sequence that has been physically mapped. A marker can be a DNA marker (detects DNA polymorphisms), a protein (detects variation at an encoded polypeptide), or a simply inherited phenotype (such as the ‘waxy’ phenotype). A DNA marker can be developed from genomic nucleotide sequence or from expressed nucleotide sequences (e.g., from a spliced RNA or a cDNA). Depending on the DNA marker technology, the marker may consist of complementary primers flanking the locus and/or complementary probes that hybridize to polymorphic alleles at the locus. The term marker locus is the locus (gene, sequence or nucleotide) that the marker detects. “Marker” or “molecular marker” or “marker locus” may also be used to denote a nucleic acid or amino acid sequence that is sufficiently unique to characterize a specific locus on the genome. Any detectable polymorphic trait can be used as a marker so long as it is inherited differentially and exhibits linkage disequilibrium with a phenotypic trait of interest. Markers that detect genetic polymorphisms between members of a population are well-established in the art. Markers can be defined by the type of polymorphism that they detect and also the marker technology used to detect the polymorphism. Marker types include but are not limited to, e.g., detection of restriction fragment length polymorphisms (RFLP), detection of isozyme markers, randomly amplified polymorphic DNA (RAPD), amplified fragment length polymorphisms (AFLPs), detection of simple sequence repeats (SSRs), detection of amplified variable sequences of the plant genome, detection of self-sustained sequence replication, or detection of single nucleotide polymorphisms (SNPs). SNPs can be detected e.g. via DNA sequencing, PCR-based sequence specific amplification methods, detection of polynucleotide polymorphisms by allele specific hybridization (ASH), dynamic allele-specific hybridization (DASH), molecular beacons, microarray hybridization, oligonucleotide ligase assays, Flap endonucleases, 5′ endonucleases, primer extension, single strand conformation polymorphism (SSCP) or temperature gradient gel electrophoresis (TGGE). DNA sequencing, such as the pyrosequencing technology has the advantage of being able to detect a series of linked SNP alleles that constitute a haplotype. Haplotypes tend to be more informative (detect a higher level of polymorphism) than SNPs.

A “marker allele”, alternatively an “allele of a marker locus”, can refer to one of a plurality of polymorphic nucleotide sequences found at a marker locus in a population. With regard to a SNP marker, allele refers to the specific nucleotide base present at that SNP locus in that individual plant.

“Fine-mapping” refers to methods by which the position of a QTL can be determined more accurately (narrowed down) and by which the size of the introgression fragment comprising the QTL is reduced. For example Near Isogenic Lines for the QTL (QTL-NILs) can be made, which contain different, overlapping fragments of the introgression fragment within an otherwise uniform genetic background of the recurrent parent. Such lines can then be used to map on which fragment the QTL is located and to identify a line having a shorter introgression fragment comprising the QTL.

“Marker assisted selection” (of MAS) is a process by which individual plants are selected based on marker genotypes. “Marker assisted counter-selection” is a process by which marker genotypes are used to identify plants that will not be selected, allowing them to be removed from a breeding program or planting. Marker assisted selection uses the presence of molecular markers, which are genetically linked to a particular locus or to a particular chromosome region (e.g. introgression fragment, transgene, polymorphism, mutation, etc), to select plants for the presence of the specific locus or region (introgression fragment, transgene, polymorphism, mutation, etc). For example, a molecular marker genetically linked to a digestibility QTL as defined herein, can be used to detect and/or select plants comprising the QTL on chromosome 7. The closer the genetic linkage of the molecular marker to the locus (e.g. about 7 cM, 6 cM, 5 cM, 4 cM, 3 cM, 2 cM, 1 cM, 0.5 cM or less), the less likely it is that the marker is dissociated from the locus through meiotic recombination. Likewise, the closer two markers are linked to each other (e.g. within 7 or 5 cM, 4 cM, 3 cM, 2 cM, 1 cM or less) the less likely it is that the two markers will be separated from one another (and the more likely they will co-segregate as a unit). A marker “within 7 cM or within 5 cM, 3 cM, 2 cM, or 1 cM” of another marker refers to a marker which genetically maps to within the 7 cM or 5 cM, 3 cM, 2 cM, or 1 cM region flanking the marker (i.e. either side of the marker). Similarly, a marker within 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less of another marker refers to a marker which is physically located within the 5 Mb, 3 Mb, 2.5 Mb, 2 Mb, 1 Mb, 0.5 Mb, 0.4 Mb, 0.3 Mb, 0.2 Mb, 0.1 Mb, 50 kb, 20 kb, 10 kb, 5 kb, 2 kb, 1 kb or less, of the genomic DNA region flanking the marker (i.e. either side of the marker). “LOD-score” (logarithm (base 10) of odds) refers to a statistical test often used for linkage analysis in animal and plant populations. The LOD score compares the likelihood of obtaining the test data if the two loci (molecular marker loci and/or a phenotypic trait locus) are indeed linked, to the likelihood of observing the same data purely by chance. Positive LOD scores favor the presence of linkage and a LOD score greater than 3.0 is considered evidence for linkage. A LOD score of +3 indicates 1000 to 1 odds that the linkage being observed did not occur by chance.

A “marker haplotype” refers to a combination of alleles at a marker locus.

A “marker locus” is a specific chromosome location in the genome of a species where a specific marker can be found. A marker locus can be used to track the presence of a second linked locus, e.g., one that affects the expression of a phenotypic trait. For example, a marker locus can be used to monitor segregation of alleles at a genetically or physically linked locus.

A “marker probe” is a nucleic acid sequence or molecule that can be used to identify the presence of a marker locus, e.g., a nucleic acid probe that is complementary to a marker locus sequence, through nucleic acid hybridization. Marker probes comprising 30 or more contiguous nucleotides of the marker locus (“all or a portion” of the marker locus sequence) may be used for nucleic acid hybridization. Alternatively, in some aspects, a marker probe refers to a probe of any type that is able to distinguish (i.e., genotype) the particular allele that is present at a marker locus.

The term “molecular marker” may be used to refer to a genetic marker or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a vis a plant without the insertion. Thus, the marker need only indicate whether the indel region is present or absent. Any suitable marker detection technology may be used to identify such a hybridization marker, e.g. SNP technology is used in the examples provided herein.

“Genetic markers” are nucleic acids that are polymorphic in a population and where the alleles of which can be detected and distinguished by one or more analytic methods, e.g., RFLP, AFLP, isozyme, SNP, SSR, and the like. The terms “molecular marker” and “genetic marker” are used interchangeably herein. The term also refers to nucleic acid sequences complementary to the genomic sequences, such as nucleic acids used as probes. Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD).

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”. The term “indel” refers to an insertion or deletion, wherein one line may be referred to as having an inserted nucleotide or piece of DNA relative to a second line, or the second line may be referred to as having a deleted nucleotide or piece of DNA relative to the first line.

“Physical distance” between loci (e.g. between molecular markers and/or between phenotypic markers) on the same chromosome is the actually physical distance expressed in bases or base pairs (bp), kilo bases or kilo base pairs (kb) or megabases or mega base pairs (Mb).

“Genetic distance” between loci (e.g. between molecular markers and/or between phenotypic markers) on the same chromosome is measured by frequency of crossing-over, or recombination frequency (RF) and is indicated in centimorgans (cM). One cM corresponds to a recombination frequency of 1%. If no recombinants can be found, the RF is zero and the loci are either extremely close together physically or they are identical. The further apart two loci are, the higher the RF.

A “physical map” of the genome is a map showing the linear order of identifiable landmarks (including genes, markers, etc.) on chromosome DNA. However, in contrast to genetic maps, the distances between landmarks are absolute (for example, measured in base pairs or isolated and overlapping contiguous genetic fragments) and not based on genetic recombination (that can vary in different populations).

An allele “negatively” correlates with a trait when it is linked to it and when presence of the allele is an indicator that a desired trait or trait form will not occur in a plant comprising the allele. An allele “positively” correlates with a trait when it is linked to it and when presence of the allele is an indicator that the desired trait or trait form will occur in a plant comprising the allele.

A centimorgan (“cM”) is a unit of measure of recombination frequency. One cM is equal to a 1% chance that a marker at one genetic locus will be separated from a marker at a second locus due to crossing over in a single generation.

As used herein, the term “chromosomal interval” designates a contiguous linear span of genomic DNA that resides in planta on a single chromosome. The genetic elements or genes located on a single chromosomal interval are physically linked. The size of a chromosomal interval is not particularly limited. In some aspects, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The term “closely linked”, in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful with respect to the subject matter of the current disclosure when they demonstrate a significant probability of co-segregation (linkage) with a desired trait (e.g., resistance to gray leaf spot). Closely linked loci such as a marker locus and a second locus can display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

“Linkage” refers to the tendency for alleles to segregate together more often than expected by chance if their transmission was independent. Typically, linkage refers to alleles on the same chromosome. Genetic recombination occurs with an assumed random frequency over the entire genome. Genetic maps are constructed by measuring the frequency of recombination between pairs of traits or markers. The closer the traits or markers are to each other on the chromosome, the lower the frequency of recombination, and the greater the degree of linkage. Traits or markers are considered herein to be linked if they generally co-segregate. A 1/100 probability of recombination per generation is defined as a genetic map distance of 1.0 centiMorgan (1.0 cM). The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency. Markers that show linkage disequilibrium are considered linked. Linked loci co-segregate more than 50% of the time, e.g., from about 51% to about 100% of the time. In other words, two markers that co-segregate have a recombination frequency of less than 50% (and by definition, are separated by less than 50 cM on the same linkage group.) As used herein, linkage can be between two markers, or alternatively between a marker and a locus affecting a phenotype. A marker locus can be “associated with” (linked to) a trait. The degree of linkage of a marker locus and a locus affecting a phenotypic trait is measured, e.g., as a statistical probability of co-segregation of that molecular marker with the phenotype (e.g., an F statistic or LOD score).

The genetic elements or genes located on a single chromosome segment are physically linked. In some embodiments, the two loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time. The genetic elements located within a chromosomal segment are also “genetically linked”, typically within a genetic recombination distance of less than or equal to 50 cM, e.g., about 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, 0.75, 0.5, 0.25 cM or less. That is, two genetic elements within a single chromosomal segment undergo recombination during meiosis with each other at a frequency of less than or equal to about 50%, e.g., about 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1%, 0.75%, 0.5%, 0.25% or less. “Closely linked” markers display a cross over frequency with a given marker of about 10% or less, e.g., 9%, 8%, 7%, 6%, 5%, 4%, 3%, 2%, 1%, 0.75%, 0.5%, 0.25% or less (the given marker locus is within about 10 cM of a closely linked marker locus, e.g., 9, 8, 7, 6, 5, 4, 3, 2, 1, 0.75, 0.5, 0.25 cM or less of a closely linked marker locus). Put another way, closely linked marker loci co-segregate at least about 90% the time, e.g., 91%, 92%, 93%, 94%, 95%, 96%, 97%, 98%, 99%, 99.5%, 99.75%, or more of the time.

As used herein, the term “sequence identity” refers to the degree of identity between any given nucleic acid sequence and a target nucleic acid sequence. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (Bl2seq) program from the stand-alone version of BLASTZ containing BLASTN and BLASTP. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the B12seq program can be found in the readme file accompanying BLASTZ. B12seq performs a comparison between two sequences using either the BLASTN or BLASTP algorithm.

BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq I .txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. The following command will generate an output file containing a comparison between two sequences: C:\B12seq -i c:\seql .txt -j c:\seq2.txt -p blastn -o c:\output.txt -q -1 -r 2. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides from the target sequence presented in alignment with the sequence from the identified sequence starting with any matched position and ending with any other matched position. A matched position is any position where an identical nucleotide is presented in both the target and identified sequences. Gaps presented in the target sequence are not counted since gaps are not nucleotides. Likewise, gaps presented in the identified sequence are not counted since target sequence nucleotides are counted, not nucleotides from the identified sequence. The percent identity over a particular length is determined by counting the number of matched positions over that length and dividing that number by the length followed by multiplying the resulting value by 100. For example, if (i) a 500-base nucleic acid target sequence is compared to a subject nucleic acid sequence, (ii) the B12seq program presents 200 bases from the target sequence aligned with a region of the subject sequence where the first and last bases of that 200-base region are matches, and (iii) the number of matches over those 200 aligned bases is 180, then the 500-base nucleic acid target sequence contains a length of 200 and a sequence identity over that length of 90% (i.e., 180/200×100=90). It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

An “isolated nucleic acid sequence” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule. A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

When reference is made to a nucleic acid sequence (e.g. DNA or genomic DNA) having “substantial sequence identity to” a reference sequence or having a sequence identity of at least 80%>, e.g. at least 85%, 90%, 95%, 98%> or 99%> nucleic acid sequence identity to a reference sequence, in one embodiment said nucleotide sequence is considered substantially identical to the given nucleotide sequence and can be identified using stringent hybridisation conditions. In another embodiment, the nucleic acid sequence comprises one or more mutations compared to the given nucleotide sequence but still can be identified using stringent hybridisation conditions. “Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

In an aspect, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and/or B, wherein molecular markers A and B are SNPs which are respectively C corresponding to position 125861690 and A corresponding to position 126109267 or which are respectively T corresponding to position 125861690 and G corresponding to position 126109267, referenced to the B73 reference genome AGPv2, optionally wherein said QTL allele is flanked by molecular markers A and/or B; or screening for the presence of molecular markers A and/or B.

In an aspect, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and/or B, wherein molecular markers A and B are SNPs which are respectively C corresponding to position 125861690 and A corresponding to position 126109267, referenced to the B73 reference genome AGPv2, optionally wherein said QTL allele is flanked by molecular markers A and/or B; or screening for the presence of molecular markers A and/or B.

In an aspect, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and/or B, wherein molecular markers A and B are SNPs which are respectively T corresponding to position 125861690 and G corresponding to position 126109267, referenced to the B73 reference genome AGPv2, optionally wherein said QTL allele is flanked by molecular markers A and/or B; or screening for the presence of molecular markers A and/or B.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular marker A, optionally wherein said QTL allele is flanked by molecular marker A; or screening for the presence of molecular marker A.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular marker B, optionally wherein said QTL allele is flanked by molecular marker B; or screening for the presence of molecular marker B.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and B, optionally wherein said QTL allele is flanked by molecular markers A and B; or screening for the presence of molecular markers A and B.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular marker A, wherein molecular marker A is a SNP which is C corresponding to position 125861690 or which is T corresponding to position 125861690, referenced to the B73 reference genome AGPv2, optionally wherein said QTL allele is flanked by molecular marker A; or screening for the presence of molecular marker A.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular marker B, wherein molecular marker B is a SNP which is A corresponding to position 126109267 or which is G corresponding to position 126109267, referenced to the B73 reference genome AGPv2, optionally wherein said QTL allele is flanked by molecular marker B; or screening for the presence of molecular marker B.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and B, wherein molecular markers A and B are SNPs which are respectively C corresponding to position 125861690 and A corresponding to position 126109267 or which are respectively T corresponding to position 125861690 and G corresponding to position 126109267, referenced to the B73 reference genome AGPv2, optionally wherein said QTL allele is flanked by molecular markers A and B; or screening for the presence of molecular markers A and B.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular marker A, wherein molecular marker A is a SNP which is C corresponding to position 125861690, referenced to the B73 reference genome AGPv2, optionally wherein said QTL allele is flanked by molecular marker A; or screening for the presence of molecular marker A.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular marker B, wherein molecular marker B is a SNP which is A corresponding to position 126109267, referenced to the B73 reference genome AGPv2, optionally wherein said QTL allele is flanked by molecular marker B; or screening for the presence of molecular marker B.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and B, wherein molecular markers A and B are SNPs which are respectively C corresponding to position 125861690 and A corresponding to position 126109267, referenced to the B73 reference genome AGPv2, optionally wherein said QTL allele is flanked by molecular markers A and B; or screening for the presence of molecular markers A and B.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular marker A, wherein molecular marker A is a SNP which is T corresponding to position 125861690, referenced to the B73 reference genome AGPv2, optionally wherein said QTL allele is flanked by molecular marker A; or screening for the presence of molecular marker A.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular marker B, wherein molecular marker B is a SNP which is G corresponding to position 126109267, referenced to the B73 reference genome AGPv2, optionally wherein said QTL allele is flanked by molecular marker B; or screening for the presence of molecular marker B.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and B, wherein molecular markers A and B are SNPs which are respectively T corresponding to position 125861690 and G corresponding to position 126109267, referenced to the B73 reference genome AGPv2, optionally wherein said QTL allele is flanked by molecular markers A and B; or screening for the presence of molecular markers A and B.

In an aspect, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and/or F, wherein molecular markers A and F are SNPs which are respectively C corresponding to position 125861690 and C corresponding to position 130881551 or which are respectively T corresponding to position 125861690 and T corresponding to position 130881551, referenced to the B73 reference genome AGPv2, optionally wherein said QTL allele is flanked by molecular markers A and/or F; or screening for the presence of molecular markers A and/or F.

In an aspect, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and/or F, wherein molecular markers A and F are SNPs which are respectively C corresponding to position 125861690 and C corresponding to position 130881551, referenced to the B73 reference genome AGPv2, optionally wherein said QTL allele is flanked by molecular markers A and/or F; or screening for the presence of molecular markers A and/or F.

In an aspect, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and/or F, wherein molecular markers A and F are SNPs which are respectively T corresponding to position 125861690 and T corresponding to position 130881551, referenced to the B73 reference genome AGPv2, optionally wherein said QTL allele is flanked by molecular markers A and/or F; or screening for the presence of molecular markers A and/or F.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular marker F, optionally wherein said QTL allele is flanked by molecular marker F; or screening for the presence of molecular marker F.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and F, optionally wherein said QTL allele is flanked by molecular markers A and F; or screening for the presence of molecular markers A and F.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular marker F, wherein molecular marker F is a SNP which is C corresponding to position 130881551 or which is T corresponding to position 130881551, referenced to the B73 reference genome AGPv2, optionally wherein said QTL allele is flanked by molecular marker F; or screening for the presence of molecular marker F.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and F, wherein molecular markers A and F are SNPs which are respectively C corresponding to position 125861690 and C corresponding to position 130881551 or which are respectively T corresponding to position 125861690 and T corresponding to position 130881551, referenced to the B73 reference genome AGPv2, optionally wherein said QTL allele is flanked by molecular markers A and F; or screening for the presence of molecular markers A and F.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular marker B, wherein molecular marker B is a SNP which is A corresponding to position 126109267, referenced to the B73 reference genome AGPv2, optionally wherein said QTL allele is flanked by molecular marker B; or screening for the presence of molecular marker B.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and F, wherein molecular markers A and F are SNPs which are respectively C corresponding to position 125861690 and C corresponding to position 130881551, referenced to the B73 reference genome AGPv2, optionally wherein said QTL allele is flanked by molecular markers A and F; or screening for the presence of molecular markers A and F.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular marker F, wherein molecular marker F is a SNP which is T corresponding to position 130881551, referenced to the B73 reference genome AGPv2, optionally wherein said QTL allele is flanked by molecular marker F; or screening for the presence of molecular marker F.

In an embodiment, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A and B, wherein molecular markers A and F are SNPs which are respectively T corresponding to position 125861690 and T corresponding to position 130881551, referenced to the B73 reference genome AGPv2, optionally wherein said QTL allele is flanked by molecular markers A and F; or screening for the presence of molecular markers A and F.

In an aspect, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers C, D, and/or E, wherein molecular markers C, D, and E are SNPs which are respectively A corresponding to position 125976029, A corresponding to position 127586792, and C corresponding to position 129887276, or which are respectively G corresponding to position 125976029, G corresponding to position 127586792, T corresponding to position 129887276, referenced to the B73 reference genome AGPv2; or screening for the presence of molecular markers C, D, and/or E.

In an aspect, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers C, D, and/or E, wherein molecular markers C, D, and E are SNPs which are respectively A corresponding to position 125976029, A corresponding to position 127586792, and C corresponding to position 129887276, referenced to the B73 reference genome AGPv2; or screening for the presence of molecular markers C, D, and/or E.

In an aspect, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers C, D, and/or E, wherein molecular markers C, D, and E are SNPs which are respectively G corresponding to position 125976029, G corresponding to position 127586792, T corresponding to position 129887276, referenced to the B73 reference genome AGPv2; or screening for the presence of molecular markers C, D, and/or E.

In certain embodiments, the QTL allele comprises molecular markers A, B, C, D, E, and/or F, preferably all.

In certain embodiments, the QTL allele comprises molecular marker A. In certain embodiments, the QTL allele comprises molecular marker B. In certain embodiments, the QTL allele comprises molecular marker C. In certain embodiments, the QTL allele comprises molecular marker D. In certain embodiments, the QTL allele comprises molecular marker E. In certain embodiments, the QTL allele comprises molecular marker F.

In certain embodiments, molecular marker alleles A, B, C, D, E, and F are as provided in Table A.

TABLE A

| Marker | | | | | | SEQ ID |
|---|---|---|---|---|---|---|
| ID | Chr | AGPv04 | AGPv02 | A_Call | B_Call | NO: |
| A | 7 | 129798239 | 125861690 | cyt | thy | 50 |
| B | 7 | 129919413 | 125976029 | ade | gua | 52 |
| C | 7 | 130053680 | 126109267 | ade | gua | 51 |
| D | 7 | 131558094 | 127586792 | ade | gua | 53 |
| E | 7 | 133928553 | 129887276 | cyt | thy | 54 |
| F | 7 | 134903902 | 130881551 | cyt | thy | 55 |

In certain embodiments, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A, B, C, D, E, and/or F, preferably all; or screening for the presence of molecular markers A, B, C, D, E, and/or F.

In certain embodiments, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A, B, C, D, E, and/or F, preferably all; or screening for the presence of molecular markers A, B, C, D, E, and/or F; wherein molecular markers A, B, C, D, E, and F are SNPs which are respectively C corresponding to position 125861690, A corresponding to position 126109267, A corresponding to position 125976029, A corresponding to position 127586792, C corresponding to position 129887276, and C corresponding to position 130881551, or which are respectively T corresponding to position 125861690, G corresponding to position 126109267, G corresponding to position 125976029, G corresponding to position 127586792, T corresponding to position 129887276, and T corresponding to position 130881551, referenced to the B73 reference genome AGPv2.

In certain embodiments, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A, B, C, D, E, and/or F, preferably all; or screening for the presence of molecular markers A, B, C, D, E, and/or F; wherein molecular markers A, B, C, D, E, and F are SNPs which are respectively C corresponding to position 125861690, A corresponding to position 126109267, A corresponding to position 125976029, A corresponding to position 127586792, C corresponding to position 129887276, and C corresponding to position 130881551, referenced to the B73 reference genome AGPv2.

In certain embodiments, the invention relates to a method for identifying a maize plant or plant part, comprising screening for the presence of a QTL allele located on chromosome 7 (such as in isolated genetic material from the plant or plant part), wherein said QTL allele is located on a chromosomal interval comprising molecular markers A, B, C, D, E, and/or F, preferably all; or screening for the presence of molecular markers A, B, C, D, E, and/or F; wherein molecular markers A, B, C, D, E, and F are SNPs which are respectively T corresponding to position 125861690, G corresponding to position 126109267, G corresponding to position 125976029, G corresponding to position 127586792, T corresponding to position 129887276, and T corresponding to position 130881551, referenced to the B73 reference genome AGPv2.

In certain embodiments, the methods according to the invention as described herein are methods for identifying plants (or plant parts) having increased drought resistance or tolerance.

In certain embodiments, the methods according to the invention as described herein are methods for identifying plants (or plant parts) having decreased drought resistance or tolerance.

In certain embodiments, the methods according to the invention as described herein are methods for identifying plants (or plant parts) having increased carbon isotope composition (δ13C).

In certain embodiments, the methods according to the invention as described herein are methods for identifying plants (or plant parts) having decreased carbon isotope composition (δ13C).

It will be understood that whenever reference is made herein to a particular molecular marker (allele), such as identification of a particular molecular marker (allele), the molecular marker (allele) can equally be identified based on the sequence as provided herein (e.g. the sequences as provided in Table A), as well as based on the complementary sequence (i.e. the corresponding nucleotide in the complementary DNA strand).

In certain embodiments, the methods as described herein comprise the step of isolating genetic material from the plant or plant part, such as from at least one cell of the plant or plant part.

In certain embodiments, the methods as described herein comprise the step of selecting a plant or plant part in which the QTL allele or molecular marker (allele) is present.

In certain embodiments, the methods as described herein comprise the step of isolating genetic material from the plant or plant part, such as from at least one cell of the plant or plant part and selecting a plant or plant part in which the QTL allele or molecular marker (allele) is present.

In an aspect, the invention relates to a method for identifying a maize plant or plant part, comprising (such as in isolated material from the plant or plant part) analysing the (protein and/or mRNA) expression level and/or (protein) activity and/or sequence of a gene comprised in the QTL according to the invention as defined herein. In certain embodiments, the method comprises isolating genetic material from at least one cell of the plant or plant part.

In certain embodiments, the expression level, activity, and/or sequence is compared with the expression level, activity, and/or sequence of a reference plant (part).

In certain embodiments, the expression level and/or activity is compared with a predetermined threshold expression level and/or activity. In certain embodiments, the threshold is indicative of drought resistance/tolerance and/or δ13C (e.g. above or below the threshold an increased or decreased drought resistance/tolerance is attributed).

In certain embodiments, the expression level and/or activity is compared between different conditions, such as control conditions and drought conditions.

In an aspect, the invention relates to a method for generating or modifying a maize plant, comprising altering the expression level and/or activity of one or more genes comprised in the QTL according to the invention as described herein. Methods for altering expression and/or activity of genes are described herein elsewhere (e.g. siRNA, knock-out, genome editing, transcriptional or translational control, mutagenesis, overexpression, etc.), and are known in the art. The skilled person will understand that expression level and/or activity can be modified constitutively or conditionally and/or can be modified selectively (e.g. tissue specific) or in the entire plant.

In certain embodiments, the expression and/or activity of the gene is reduced, such as at least 10%, preferably at least 20%, more preferably at least 50%.

In certain embodiments, the expression level and/or activity of the gene is increased, such as at least 10%, preferably at least 20%, more preferably at least 50%.

In certain embodiments, the gene is mutated. In certain embodiments the mutation alters expression of the wild type or native protein and/or mRNA. In certain embodiments the mutation reduces or eliminates expression of the (wild type or native) protein and/or mRNA, as described herein elsewhere. Mutations may affect transcription and/or translation. Mutations may occur in exons or introns. Mutations may occur in regulatory elements, such as promotors, enhancers, terminators, insulators, etc. Mutations may occur in coding sequences. Mutations may occur in splicing signal sites, such as splice donor or splice acceptor sites. Mutations may be frame shift mutations. Mutations may be nonsense mutations. Mutations may be insertion or deletion of one or more nucleotides. Mutations may be non-conservative mutations (in which one or more wild type amino acids are replaced with one or more non-wild type amino acids). Mutations may affect or alter the function of the protein, such as enzymatic activity. Mutations may reduce or (substantially) eliminate the function of the protein, such as enzymatic activity. Reduced function, such as reduced enzymatic activity, may refer to a reduction of about at least 10%, preferably at least 30%, more preferably at least 50%, such as at least 20%, 40%, 60%, 80% or more, such as at least 85%, at least 90%, at least 95%, or more. (Substantially) eliminated function, such as (substantially) eliminated enzymatic activity, may refer to a reduction of at least 80%, preferably at least 90%, more preferably at least 95%. Mutations may be dominant negative mutations.

In certain embodiments, the mutation is an insertion of one or more nucleotides in the coding sequence. In certain embodiments, the mutation is a nonsense mutation. In certain embodiments, the mutation results in altered expression of the gene. In certain embodiments, the mutation results in knockout of the gene or knockdown of the mRNA and/or protein. In certain embodiments, the mutation results in a frame shift of the coding sequence of. In certain embodiments, the mutation results in an altered protein sequence encoded by the gene.

mRNA and/or protein expression may be reduced or eliminated by mutating the gene itself (including coding, non-coding, and regulatory element). Methods for introducing mutations are described herein elsewhere. Alternatively, mRNA and/or protein expression may be reduced or eliminated by (specifically) interfering with transcription and/or translation, such as to decrease or eliminate mRNA and/or protein transcription or translation. Alternatively, mRNA and/or protein expression may be reduced or eliminated by (specifically) interfering with mRNA and/or protein stability, such as to reduce mRNA and/or protein stability. By means of example, mRNA (stability) may be reduced by means of RNAi, as described herein elsewhere. Also miRNA can be used to affect mRNA (stability). In certain embodiments, a reduced expression which is achieved by reducing mRNA or protein stability is also encompassed by the term “mutated”. In certain embodiments, a reduced expression which is achieved by reducing mRNA or protein stability is not encompassed by the term “mutated”.

In certain embodiments, the expression level and/or activity of the gene is increased by overexpression, such as transgenic overexpression or overexpression resulting from transcriptional and/or translational control, as is known in the art. Overexpression may result from increase in copy number.

In an aspect, the invention relates to a method for generating or modifying a maize plant, comprising introducing into the (genome of the) plant the QTL according to the invention as described herein. Methods for introducing the QTL are described herein elsewhere (e.g. transgenesis, introgression, etc), and are known in the art. The skilled person will understand that the QTL may be introduced in the germline or alternatively may be introduced tissue-specific.

In an aspect, the invention relates to a maize plant or plant part modified or generated as such. In certain embodiments, the plant is not a plant variety.

In an aspect, the invention relates to a maize plant or plant part comprising the QTL according to the invention or one or more molecular marker alleles according to the invention as described herein (such as molecular marker alleles A and/or B, or A and/or F, A, B, C, D, E, and/or F, preferably all).

In certain embodiments, the gene comprised in the QTL according to the invention as described herein is selected from Abh4, CSLE1, WEB1, GRMZM2G397260, and Hsftf21.

In certain embodiments Abh4 is selected from
(i) a nucleotide sequence comprising the sequence of SEQ ID NO: 9 or 18;
(ii) a nucleotide sequence having the cDNA of SEQ ID NO: 11, 14, 17, or 20;
(iii) a nucleotide sequence encoding for an amino acid sequence having the amino acid sequence of SEQ ID NO: 12, 15, or 21;
(iv) a nucleotide sequence having at least 60%, preferably at least 80%, more preferably at least 90%, most preferably at least 95%, such as at least 98% identity to the sequence of SEQ ID NO: 9, 11, 14, 17, 18, or 20;
(v) a nucleotide sequence encoding for a polypeptide having at least 60%, preferably at least 80%, more preferably at least 90%, most preferably at least 95%, such as at least 98% identity to the sequence of SEQ ID NO: 12, 15, or 21;
(vi) a nucleotide sequence hybridizing with the reverse complement of a nucleotide sequence as defined in (i), (ii) or (iii) under stringent hybridization conditions; and
(vii) a nucleotide sequence encoding a protein derived from the amino acid sequence encoded by the nucleotide sequence of (i) to (vi) by way of substitution, deletion and/or addition of one or more amino acid(s).

In certain embodiments CSLE1 is selected from
(i) a nucleotide sequence comprising the sequence of SEQ ID NO: 1 or 4;
(ii) a nucleotide sequence having the cDNA of SEQ ID NO: 2 or 5;
(iii) a nucleotide sequence encoding for an amino acid sequence having the amino acid sequence of SEQ ID NO: 3 or 6;
(iv) a nucleotide sequence having at least 60%, preferably at least 80%, more preferably at least 90%, most preferably at least 95%, such as at least 98% identity to the sequence of SEQ ID NO: 1, 2, 4, or 5;
(v) a nucleotide sequence encoding for a polypeptide having at least 60%, preferably at least 80%, more preferably at least 90%, most preferably at least 95%, such as at least 98% identity to the sequence of SEQ ID NO: 3 or 6;
(vi) a nucleotide sequence hybridizing with the reverse complement of a nucleotide sequence as defined in (i), (ii) or (iii) under stringent hybridization conditions; and
(vii) a nucleotide sequence encoding a protein derived from the amino acid sequence encoded by the nucleotide sequence of (i) to (vi) by way of substitution, deletion and/or addition of one or more amino acid(s).

In certain embodiments WEB1 is selected from
(i) a nucleotide sequence comprising the sequence of SEQ ID NO: 24 or 27;
(ii) a nucleotide sequence having the cDNA of SEQ ID NO: 25 or 28;
(iii) a nucleotide sequence encoding for an amino acid sequence having the amino acid sequence of SEQ ID NO: 26 or 29;
(iv) a nucleotide sequence having at least 60%%, preferably at least 80%, more preferably at least 90%, most preferably at least 95%, such as at least 98% identity to the sequence of SEQ ID NO: 24, 25, 27, or 28;
(v) a nucleotide sequence encoding for a polypeptide having at least 60%%, preferably at least 80%, more preferably at least 90%, most preferably at least 95%, such as at least 98% identity to the sequence of SEQ ID NO: 26 or 29;
(vi) a nucleotide sequence hybridizing with the reverse complement of a nucleotide sequence as defined in (i), (ii) or (iii) under stringent hybridization conditions; and
(vii) a nucleotide sequence encoding a protein derived from the amino acid sequence encoded by the nucleotide sequence of (i) to (vi) by way of substitution, deletion and/or addition of one or more amino acid(s).

In certain embodiments GRMZM2G397260 is selected from
(i) a nucleotide sequence comprising the sequence of SEQ ID NO: 32;
(ii) a nucleotide sequence having the cDNA of SEQ ID NO: 33;
(iii) a nucleotide sequence encoding for an amino acid sequence having the amino acid sequence of SEQ ID NO: 34;
(iv) a nucleotide sequence having at least 60%%, preferably at least 80%, more preferably at least 90%, most preferably at least 95%, such as at least 98% identity to the sequence of SEQ ID NO: 32 or 33;
(v) a nucleotide sequence encoding for a polypeptide having at least 60%%, preferably at least 80%, more preferably at least 90%, most preferably at least 95%, such as at least 98% identity to the sequence of SEQ ID NO: 34;
(vi) a nucleotide sequence hybridizing with the reverse complement of a nucleotide sequence as defined in (i), (ii) or (iii) under stringent hybridization conditions; and
(vii) a nucleotide sequence encoding a protein derived from the amino acid sequence encoded by the nucleotide sequence of (i) to (vi) by way of substitution, deletion and/or addition of one or more amino acid(s).

In certain embodiments Hsftf21 is selected from
(i) a nucleotide sequence comprising the sequence of SEQ ID NO: 36 or 39;
(ii) a nucleotide sequence having the cDNA of SEQ ID NO: 37 or 40;
(iii) a nucleotide sequence encoding for an amino acid sequence having the amino acid sequence of SEQ ID NO: 38 or 41;
(iv) a nucleotide sequence having at least 60%%, preferably at least 80%, more preferably at least 90%, most preferably at least 95%, such as at least 98% identity to the sequence of SEQ ID NO: 36, 37, 39, or 40;
(v) a nucleotide sequence encoding for a polypeptide having at least 60%%, preferably at least 80%, more preferably at least 90%, most preferably at least 95%, such as at least 98% identity to the sequence of SEQ ID NO: 38 or 41;
(vi) a nucleotide sequence hybridizing with the reverse complement of a nucleotide sequence as defined in (i), (ii) or (iii) under stringent hybridization conditions; and
(vii) a nucleotide sequence encoding a protein derived from the amino acid sequence encoded by the nucleotide sequence of (i) to (vi) by way of substitution, deletion and/or addition of one or more amino acid(s).

In certain embodiments, if the (protein and/or mRNA) expression level or activity of the gene or genes comprised in the QTL according to the invention as described herein is reduced or expression is (substantially) absent or eliminated, then the plant or plant part has increased drought resistance or tolerance. In certain embodiments, if the (protein and/or mRNA) expression level or activity of the gene or genes comprised in the QTL according to the invention as described herein is reduced or expression is (substantially) absent or eliminated compared to a reference expression level, then the plant or plant part has increased drought resistance or tolerance. In certain embodiments, if the (protein and/or mRNA) expression level or activity of the gene or genes comprised in the QTL according to the invention as described herein is reduced or expression is (substantially) absent or eliminated compared to the reference expression level in a reference plant or plant part, then the plant or plant part has increased drought resistance or tolerance.

In certain embodiments, if the (protein and/or mRNA) expression level or activity of the gene or genes comprised in the QTL according to the invention as described herein is increased, then the plant or plant part has increased drought resistance or tolerance. In certain embodiments, if the (protein and/or mRNA) expression level or activity of the gene or genes comprised in the QTL according to the invention as described herein is increased compared to a reference expression level, then the plant or plant part has increased drought resistance or tolerance. In certain embodiments, if the (protein and/or mRNA) expression level or activity of the gene or genes comprised in the QTL according to the invention as described herein is increased compared to the reference expression level in a reference plant or plant part, then the plant or plant part has increased drought resistance or tolerance.

In certain embodiments, if the (protein and/or mRNA) expression level or activity of the gene or genes comprised in the QTL according to the invention as described herein is reduced or expression is (substantially) absent or eliminated, then the plant or plant part has increased carbon isotope composition (δ13C). In certain embodiments, if the (protein and/or mRNA) expression level or activity of the gene or genes comprised in the QTL according to the invention as described herein is reduced or expression is (substantially) absent or eliminated compared to a reference expression level, then the plant or plant part has increased carbon isotope composition (δ13C). In certain embodiments, if the (protein and/or mRNA) expression level or activity of the gene or genes comprised in the QTL according to the invention as described herein is reduced or expression is (substantially) absent or eliminated compared to the reference expression level in a reference plant or plant part, then the plant or plant part has increased carbon isotope composition (δ13C).

In certain embodiments, if the (protein and/or mRNA) expression level or activity of the gene or genes comprised in the QTL according to the invention as described herein is increased, then the plant or plant part has increased carbon isotope composition (δ13C). In certain embodiments, if the (protein and/or mRNA) expression level or activity of the gene or genes comprised in the QTL according to the invention as described herein is increased compared to a reference expression level, then the plant or plant part has increased carbon isotope composition (δ13C). In certain embodiments, if the (protein and/or mRNA) expression level or activity of the gene or genes comprised in the QTL according to the invention as described herein is increased compared to the reference expression level in a reference plant or plant part, then the plant or plant part has increased carbon isotope composition (δ13C).

In certain embodiments, the (protein and/or mRNA) expression level and/or (protein) activity of Abh4 is increased. In certain embodiments, the (protein and/or mRNA) expression level and/or (protein) activity of Abh4 is decreased.

In certain embodiments, the (protein and/or mRNA) expression level and/or (protein) activity of CSLE1 is increased. In certain embodiments, the (protein and/or mRNA) expression level and/or (protein) activity of CSLE1 is decreased.

In certain embodiments, the (protein and/or mRNA) expression level and/or (protein) activity of WEB1 is increased. In certain embodiments, the (protein and/or mRNA) expression level and/or (protein) activity of WEB1 is decreased.

In certain embodiments, the (protein and/or mRNA) expression level and/or (protein) activity of GRMZM2G397260 is increased. In certain embodiments, the (protein and/or mRNA) expression level and/or (protein) activity of GRMZM2G397260 is decreased.

In certain embodiments, the (protein and/or mRNA) expression level and/or (protein) activity of Hsftf21 is increased. In certain embodiments, the (protein and/or mRNA) expression level and/or (protein) activity of Hsftf21 is decreased.

Methods for screening for the presence of a QTL allele or (molecular) marker allele as described herein are known in the art. Without limitation, screening may encompass or comprise sequencing, hybridization based methods (such as (dynamic) allele-specific hybridization, molecular beacons, SNP microarrays), enzyme based methods (such as PCR, KASP (Kompetitive Allele Specific PCR), RFLP, ALFP, RAPD, Flap endonuclease, primer extension, 5′-nuclease, oligonucleotide ligation assay), post-amplification methods based on physical properties of DNA (such as single strand conformation polymorphism, temperature gradient gel electrophoresis, denaturing high performance liquid chromatography, high-resolution melting of the entire amplicon, use of DNA mismatch-binding proteins, SNPIex, surveyor nuclease assay), etc.

In certain embodiments, the QTL allele, marker allele(s), and/or mutated genes or genes the expression or activity of which is altered as described herein in the first plant is present in a homozygous state. In certain embodiments the QTL allele, marker allele(s), and/or mutated genes or genes the expression or activity of which is altered in the first plant is (are) present in a heterozygous state. In certain embodiments, the QTL allele, marker allele(s), and/or mutated genes or genes the expression or activity of which is altered as described herein in the second plant is (are) present in a heterozygous state. In certain embodiments the QTL allele, marker allele(s), and/or mutated genes or genes the expression or activity of which is altered as described herein in the second plant is not present.

In certain embodiments, the progeny is selected in which the QTL allele, marker allele(s), and/or mutated genes or genes the expression or activity of which is altered as described herein is (are) present in a homozygous state. In certain embodiments, the progeny is selected in which the QTL allele, marker allele(s), and/or mutated genes or genes the expression or activity of which is altered as described herein is (are) present in a heterozygous state.

In certain embodiments, the methods for obtaining plants or plant parts as described herein according to the invention, such as the methods for obtaining plants or plant parts having modified drought resistance or tolerance or modified δ13C, such as increased or decreased drought resistance or tolerance or increased or decreased δ13C, involve or comprise transgenesis and/or gene editing, such as including CRISPR/Cas, TALEN, ZFN, meganucleases; (induced) mutagenesis, which may or may not be random mutagenesis, such as TILLING. In certain embodiments, the methods for obtaining plants or plant parts as described herein according to the invention, such as the methods for obtaining plants or plant parts having modified drought resistance or tolerance or modified δ13C, such as increased or decreased drought resistance or tolerance or increased or decreased δ13C, involve or comprise RNAi applications, which may or may not be, comprise, or involve transgenic applications. By means of example, non-transgenic applications may for instance involve applying RNAi components such as double stranded siRNAs to plants or plant surfaces, such as for instance as a spray. Stable integration into the plant genome is not required.

In certain embodiments, the methods for obtaining plants or plant parts as described herein according to the invention, such as the methods for obtaining plants or plant parts having modified drought resistance or tolerance or modified δ13C, such as increased or decreased drought resistance or tolerance or increased or decreased δ13C, do not involve or comprise transgenesis, gene editing, and/or mutagenesis.

In certain embodiments, the methods for obtaining plants or plant parts as described herein according to the invention, such as the methods for obtaining plants or plant parts having modified drought resistance or tolerance or modified δ13C, such as increased or decreased drought resistance or tolerance or increased or decreased δ13C, involve, comprise or consist of breeding and selection.

In certain embodiments, the methods for obtaining plants or plant parts as described herein according to the invention, such as the methods for obtaining plants or plant parts having modified drought resistance or tolerance or modified δ13C, such as increased or decreased drought resistance or tolerance or increased or decreased δ13C, do not involve, comprise or consist of breeding and selection.

In an aspect, the invention relates to a plant or plant part obtained or obtainable by the methods of the invention as described herein, such as the methods for obtaining plants or plant parts having modified drought resistance or tolerance or modified δ13C, such as increased or decreased drought resistance or tolerance or increased or decreased δ13C.

In an aspect, the invention relates to the use of one or more of the (molecular) markers described herein for identifying a plant or plant part, such as a plant or plant part having modified drought resistance or tolerance or modified δ13C, such as increased or decreased drought resistance or tolerance or increased or decreased δ13C. In an aspect, the invention relates to the use of one or more of the (molecular) markers described herein which are able to detect at least one diagnostic marker allele for identifying a plant or plant part, such as a plant or plant part having modified drought resistance or tolerance or modified δ13C, such as increased or decreased drought resistance or tolerance or increased or decreased δ13C. In an aspect, the invention relates to the detection of one or more of the (molecular) marker alleles described herein for identifying a plant or plant part, such as a plant or plant part having modified drought resistance or tolerance or modified δ13C, such as increased or decreased drought resistance or tolerance or increased or decreased δ13C.

The marker alleles of the invention as described herein may be diagnostic marker alleles which are useable for identifying plants or plant parts, such as plants or plant parts having modified drought resistance or tolerance or modified δ13C, such as increased or decreased drought resistance or tolerance or increased or decreased δ13C.

In an aspect, the invention relates to a (isolated) polynucleic acid, or the complement or the reverse complement, comprising and/or flanked by a (molecular) marker allele of the invention. In certain embodiments, the invention relates to a polynucleic acid comprising at least 10 contiguous nucleotides, preferably at least 15 contiguous nucleotides or at least 20 contiguous nucleotides of a (molecular) marker allele of the invention, or the complement or the reverse complement of a (molecular) marker allele of the invention. In certain embodiments, the polynucleic acid is capable of discriminating between a (molecular) marker allele of the invention and a non-molecular marker allele, such as to specifically hybridise with a (molecular) marker allele of the invention. It will be understood that a unique section or fragment preferably refers to a section or fragment comprising the SNP or the respective marker alleles of the invention, or a section or fragment comprising the 5′ or 3′ junction of the insert of a marker allele of the invention or a section or fraction comprised within the insert of a marker allele of the invention, or a section or fragment comprising the junction of the deletion of a marker allele of the invention.

In an aspect, the invention relates to a polynucleic acid capable of specifically hybridizing with a (molecular) marker allele of the invention, or the complement thereof, or the reverse complement thereof.

In certain embodiments, the polynucleic acid is a primer. In certain embodiments, the polynucleic acid is a probe.

In certain embodiments, the polynucleic acid is an allele specific polynucleic acid, such as an allele specific primer or probe.

In certain embodiments, the polynucleic acid comprises at least 15 nucleotides, such as 16, 17, 18, 19, 20, 21, 22, 23, 24, or 25 nucleotides, such as at least 30, 35, 40, 45, or 50 nucleotides, such as at least 100, 200, 300, or 500 nucleotides.

It will be understood that “specifically hybridizing” means that the polynucleic acid hybridises with the (molecular) marker allele (such as under stringent hybridisation conditions, as defined herein elsewhere), but does not (substantially) hybridise with a polynucleic acid not comprising the marker allele or is (substantially) incapable of being used as a PCR primer. By means of example, in a suitable readout, the hybridization signal with the marker allele or PCR amplification of the marker allele is at least 5 times, preferably at least 10 times stronger or more than the hybridisation signal with a non-marker allele, or any other sequence.

In an aspect, the invention relates to a kit comprising such polynucleic acids, such as primers (comprising forward and/or reverse primers) and/or probes. The kit may further comprise instructions for use.

In will be understood that in embodiments relating to a set of forward and reverse primers, only one of both primers (forward or reverse) may need to be capable of discriminating between a (molecular) marker allele of the invention and a non-marker allele, and hence may be unique. The other primer may or may not be capable of discriminating between a (molecular) marker allele of the invention and a non-marker allele, and hence may be unique.

The aspects and embodiments of the invention are further supported by the following non-limiting examples.

### EXAMPLES

### Example 1

The present invention describes the identification, localization and characterization of a quantitative trait locus (QTL) on maize chromosome 7 contributing among others to genetic variation in stable carbon isotope composition, stomatal conductance and plant performance under drought. This QTL is characterized on the sequence level and its phenotypic effect at the molecular, biochemical, physiological and agronomic level is described. Genes within the QTL were identified, and functional validation studies and gene expression studies are conducted as well as transgenic approaches. Molecular marker data integration and application allowed identifying positive and negative haplotypes at the locus and gene level, selecting trait carriers, and monitoring diversity at and surrounding the locus as such.

### Materials and Methods

### Development of KASP Markers

To generate new recombinants derived from the backcross of NIL B to the RP (Avramova et al. (2019). Carbon isotope composition, water use efficiency, and drought sensitivity are controlled by a common genomic segment in maize. Theoretical and Applied Genetics, 132:53-63), new molecular markers were developed. KASP markers positioned in the introgression on chromosome 7 and polymorphic between the two parental lines were generated using the publicly available 600 k Axiom™ Maize Genotyping Array (Unterseer et al., 2014) as resource.

TABLE 1
KASP markers derived from 600K array with marker information
(name, physical coordinates) and corresponding A (RP—recurrent
parent) and B allele (DP—donor parent) calls

| Marker | | | | | | SEQ ID |
|---|---|---|---|---|---|---|
| ID | Chr | AGPv04 | AGPv02 | A_Call | B_Call | NO: |
| 1 | 7 | 114162912 | 110930219 | ade | gua | 44 |
| 2 | 7 | 118512477 | 115107967 | ade | cyt | 45 |
| 3 | 7 | 121214812 | 117519226 | cyt | thy | 46 |
| 4 | 7 | 123728849 | 119973922 | ade | gua | 47 |
| 5 | 7 | 125223361 | 121316500 | gua | ade | 48 |
| 6 | 7 | 127837336 | 123827128 | cyt | ade | 49 |
| 7/A | 7 | 129798239 | 125861690 | cyt | thy | 50 |
| 8a/C | 7 | 129919413 | 125976029 | ade | gua | 51 |
| 8b/B | 7 | 130053680 | 126109267 | ade | gua | 52 |
| 9/D | 7 | 131558094 | 127586792 | ade | gua | 53 |
| 10/E | 7 | 133928553 | 129887276 | cyt | thy | 54 |
| 11/F | 7 | 134903902 | 130881551 | cyt | thy | 55 |
| 12 | 7 | 135221445 | 131191105 | thy | cyt | 56 |
| 13 | 7 | 137626045 | 133530779 | thy | cyt | 57 |
| 14 | 7 | 139623696 | 135468905 | thy | cyt | 58 |
| 15 | 7 | 141161954 | 136866388 | ade | gua | 59 |
| 16 | 7 | 148349595 | 143410578 | gua | ade | 60 |
| 17 | 7 | 151797979 | 146596371 | ade | gua | 61 |
| 18 | 7 | 155419484 | 150177783 | ade | gua | 62 |

### Development of Recombinant NILs

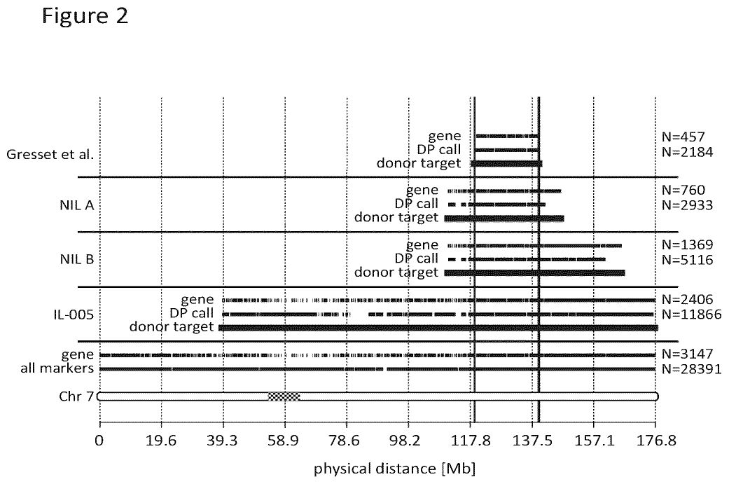

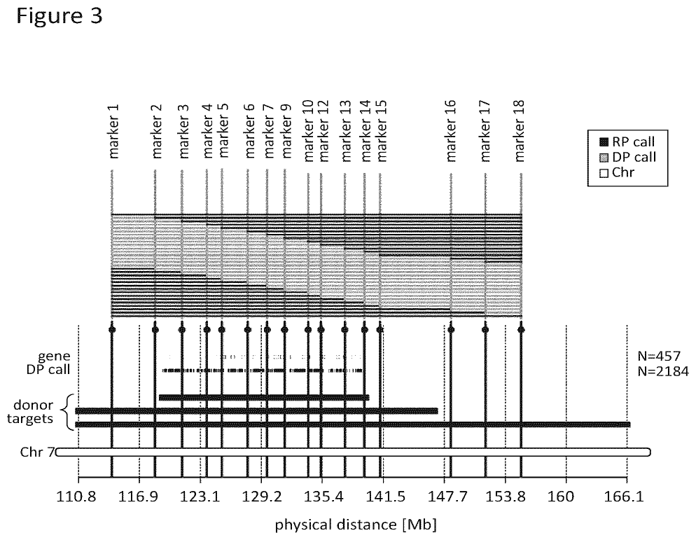

F2 plants originating from the cross of NIL B and RP were grown. After leaf tissue sampling, genotyping using KASP markers (Table 1) was carried out. Plants showing recombination in the region between marker 1 (110.930.219 bp) and marker 18 (150.177.783 bp) were selfed and seed was increased. These recombinants assisted in identifying the causal QTL fragment within the target region (FIG. 2). Recombinants were analyzed with additional DNA markers (FIG. 3) and phenotyped for iWUE (intrinsic water use efficiency), stomatal parameters and agronomic traits in a greenhouse experiment.

### RNA-Seq Analysis and Candidate Gene Extraction

An experiment with RP and DP was conducted in the glasshouse. Control (well-watered) and treatment conditions (water-withholding) were included in the experimental setup. The experiment consisted of growing RP and DP plants under controlled conditions at 29° C./21° C. day/night (d/n), 544 μmol m-2 s-1 photosynthetically active radiation (PAR), 47%/72% d/n relative humidity (RH) for a synchronization period. Subsequently, half of the plants were shifted to a drought treatment, where water was withheld for 11 days, while the other half kept growing under control conditions. Tissue samples were taken at 4, 7 and 11 days after water-withholding of watering. In addition, a recovery treatment was applied by re-watering after 11 days of drought. Each sample consisted of a mix of 3 plants per treatment and genotype. Sequencing was carried out using short read Illumina sequencing on the HiSeq2000 using paired end sequences. Read mapping was carried out using B73 AGPv02 as reference genome and applying default parameters of the CLC genomics server software suite (QIAGEN Bioinformatics, USA).

Using the AGPv02 public reference annotation (maizegdb.org/assembly), genes mapping to the target region were extracted, and if available, functional information (Protein family [PFAM] domains and gene ontology [GO] terms) was integrated for further characterization. Grouping of genes to functional protein family classes was carried out using the statistical software R with base functionalities. For gene ontology (GO) enrichment analysis the public gene annotation of the reference sequence AGPv02 was used as background set and compared to GO terms for genes mapping to the target region of 5.02 Mb. Using R together with the topGO R package, enriched GOs for cellular component, biological process and molecular function were identified using classic Fisher, Kolmogorov-Smirnoff and the Kolmogorv Smirnoff elimination test statistics. The 10 most significant GO terms (without multiple testing correction) for respective GO categories were retrieved and visualized in a node/edge GO graph using the R package Rgraphviz.

### Phenotypic Evaluation

Stomatal conductance (gs), net CO₂ assimilation rate (A), and transpiration (E) were measured for the set of recombinants D to K and parental lines at developmental stage V5 in the growth chamber under optimal conditions. Intrinsic water use efficiency (iWUE) was calculated as the ratio of A/gₛ. Significant differences between donor fragment carriers and non-carriers were determined by applying Tukey's honest significant differences test (TukeyHSD) using the statistical software R.

### Results

Marker/Phenotype Correlations within the Set of Identified Recombinants

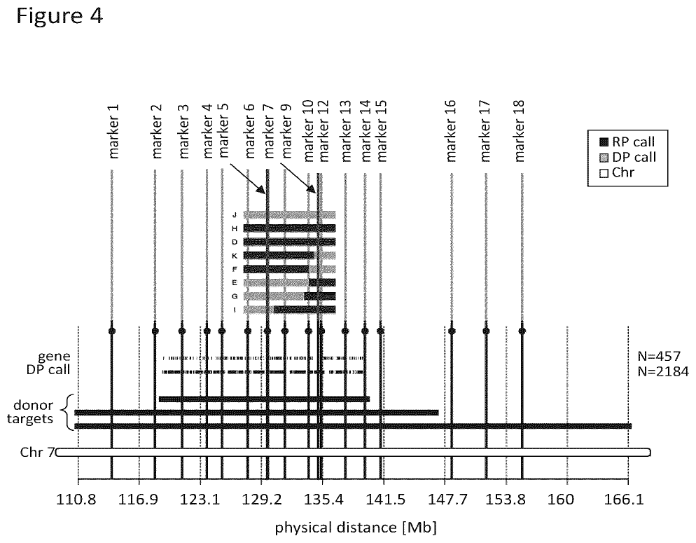

Using the newly generated KASP markers, about 2000 F2 plants were screened and recombinants J, H, D, K, F, E, G and I were selected, analyzed with additional DNA markers and characterized for phenotypic values described above. Marker/phenotype correlations showed that the 5.02 Mb target region affecting δ13C has an effect on stomatal parameters and marker 7 (125.861.690 bp) and marker 11 (130.881.551 bp) could be used as markers flanking the region (FIG. 4). The phenotypic values for selected recombinants either carrying the donor fragment (QTL+) or having RP allelic state (QTL−) at the respective genomic interval are given in Table 2. The recombinants are further characterized for other traits that showed to be controlled by the larger donor segment carried by NIL B, i.e. δ13C, leaf growth sensitivity to drought, whole plant water use efficiency (WUEplant), stomatal density, ABA leaf content.

Test statistics for the contrasting groups of genotypes carrying the positive allele at the QTL (QTL+) versus genotypes carrying the negative allele (QTL−) have been conducted. The p-value of TukeyHSD highlight a significant difference between QTL+ and QTL− genotypes for the traits gs, A, iWUE, and E. No significant difference could be detected for A. Considering the genotype information of the newly generated recombinants, the impact of the donor fragment on variation for iWUE, gₛ, A and E is substantiated with the causal difference mapping to the reduced interval of 5.02 Mb.

TABLE 2
Stomatal parameters for recombinants and parental lines as well as iWUE
values given as mean of independent plants having the same genotype
with corresponding standard deviation and presence state of the QTL

| Genotype | gₛ | A | iWUE | E | QTL |
|---|---|---|---|---|---|
| Rec D+ | 0.133 ± 0.012 | 26.778 ± 0.500 | 191.546 ± 5.352 | 0.00204 ± 0.00026 | − |
| Rec J* | 0.203 ± 0.007 | 30.827 ± 1.106 | 152.195 ± 2.155 | 0.00281 ± 0.00014 | + |
| Rec E | 0.193 ± 0.010 | 31.784 ± 0.564 | 166.001 ± 7.292 | 0.00258 ± 0.00012 | + |
| Rec F | 0.139 ± 0.006 | 26.701 ± 1.016 | 193.724 ± 4.768 | 0.00188 ± 8.48E−05 | − |
| Rec G | 0.174 ± 0.006 | 28.071 ± 0.733 | 162.127 ± 3.782 | 0.00239 ± 8.73E−05 | + |
| Rec I | 0.179 ± 0.009 | 28.785 ± 1.062 | 162.714 ± 3.890 | 0.00247 ± 0.00015 | + |
| Rec K | 0.150 ± 0.008 | 27.443 ± 0.871 | 185.446 ± 6.987 | 0.00206 ± 9.95E−05 | − |

*Rec J carries the DP haplotype in the interval and is correspondingly considered as acting like the donor genotype;
+Rec D carries the RP haplotype in the interval and is considered as acting like the recurrent parent

### Identification of Genes

Within the 5.02 Mb target region, 121 gene features can be mapped according to the AGPv02 reference annotation. Considering the PFAM domain information, the 121 gene models can be grouped into different functional classes. Beside of the 48 genes without functional information, genes within the target interval were attributed to DNA/RNA binding and transcription factor activity, as well as functions of the primary plant metabolism (e.g. carbohydrate metabolism). With hormones, cell wall and photosynthesis-related genes, pathways which might influence stomatal parameters and carbon isotope composition were found.

A GO enrichment analysis was carried out to identify GO terms that point to important pathways underlying the observed trait variation. For cellular component GO terms a significant enrichment of chloroplast-located processes manifest. In addition, nucleus and RNA splicing related processes were identified. Enrichment analysis of biological process GOs refers to abiotic stress response, fatty acid related and RNA processing pathways.

Finally, the enrichment analysis for molecular function GOs also yielded significantly enriched terms that are linked to primary metabolism, RNA/DNA modification and photosynthesis components.

Altogether, the contribution of RNA modulation/regulation and photosynthesis-related pathways on the trait variation is emphasized by the conducted analyses. For several genes located within the 5.02 Mb region, we detected differential gene expression in response to drought stress, which indicate a role for the observed phenotype.

### Validation of Genes

### ZmCSLE1

### (B73: Genomic DNA: SEQ ID NO: 1; Coding Sequence: SEQ ID NO: 2; Protein: SEQ ID NO: 3; PH207: Genomic DNA: SEQ ID NO: 4; Coding Sequence: SEQ ID NO: 5; Protein: SEQ ID NO: 6)

Based on the RNA-Seq data this gene showed a significantly different expression with higher expression in RP than in DP, with fold change (FC) of 2.044 in control conditions. Its localization on chromosome 7 from 130,735,393 to 130,740,535 bp on AGPv02 coordinates (from 134,723,714 to 134,728,829 bp on AGPv04 coordinates; from 130,675,946 to 130,681,219 bp on PH207 coordinates) makes it a positional gene. It was also one of the genes, which was downregulated under drought stress conditions more in RP (FC 5.05), compared to DP (FC 2.5). Moreover, its putative function as cellulose synthase like enzyme makes it a functional gene. Cellulose synthase enzymes are important in cell-wall synthesis, where they deliver and modify the necessary building blocks. As cell-wall synthesis processes, especially the cell-wall structure and composition, have a strong impact on transpiration and water loss, this gene might contribute to the observed trait variation. Expression differences caused by allelic variation at this locus might change stomatal parameters and/or carbohydrate relations between source and sink and thereby affect WUE and carbon isotope discrimination. A higher expression of ZmCSLE1 in donor state leads to altered carbohydrate signaling and/or differences in the hydraulic signaling of water deficit so that stomatal conductance remains high even under water stress. To validate ZmCSLE1, TILLING mutants having disrupted splicing sites, early stop codons and amino acid exchanges, were generated in a non-donor population of line PH207 (Tables 3a and 3b) and allele variants of ZmCSLE1 are tested.

TABLE 3a
Overview about the generated TILLING
mutants for the ZmCSLE1 gene model

| ZmCSLE1 | | N° mutants | | |
|---|---|---|---|---|
| Pop. PH207 | | Introns | Exons | AA Exchange |
| Winter 15/16 | Σ 18 | 3 | 15 | 11 |

TABLE 3b
Characterization of selected TILLING mutants of population
PH207. AA = amino acid, wt = wildtype, mut = mutant

| | codon | AA | Codon | AA | | position | allele | allele |
|---|---|---|---|---|---|---|---|---|
| mutant code | wt | wt | mut | mut | location | in AA seq | wt | mut |
| PH207m014a | gca | ala | aca | thr | exon 7 | 672 | G | A |
| PH207m014b | gtg | val | atg | met | exon 7 | 664 | G | A |
| PH207m014c | gcc | ala | acc | thr | exon 4 | 281 | G | A |
| PH207m014d | gtt | val | att | ile | exon 3 | 242 | G | A |
| PH207m014e | ccg | pro | ctg | leu | exon 2 | 158 | C | T |
| PH207m014f | tcc | ser | ttc | phe | exon 2 | 150 | C | T |
| PH207m014g | gtc | val | atc | ile | exon 2 | 112 | G | A |
| PH207m014h | tcg | ser | ttg | leu | exon 2 | 106 | C | T |
| PH207m014i | ctc | leu | ttc | phe | exon 1 | 84 | C | T |
| PH207m014j | ccc | pro | tcc | ser | exon 1 | 74 | C | T |
| PH207m014k | tgg | trp | tga | stop | exon 1 | 59 | G | A |

Furthermore, the analysis of the recombinants in terms of gas-exchange parameters points to a short donor segment of 248 kb ranging from marker 7 (125.861.690 bp) to marker 8b (126.109.267 bp) and harboring four genes on AGPv02. We show that this smaller interval has a specific effect on stomatal conductance and iWUE. Therefore, the four genes are described below.

### ZmAbh4

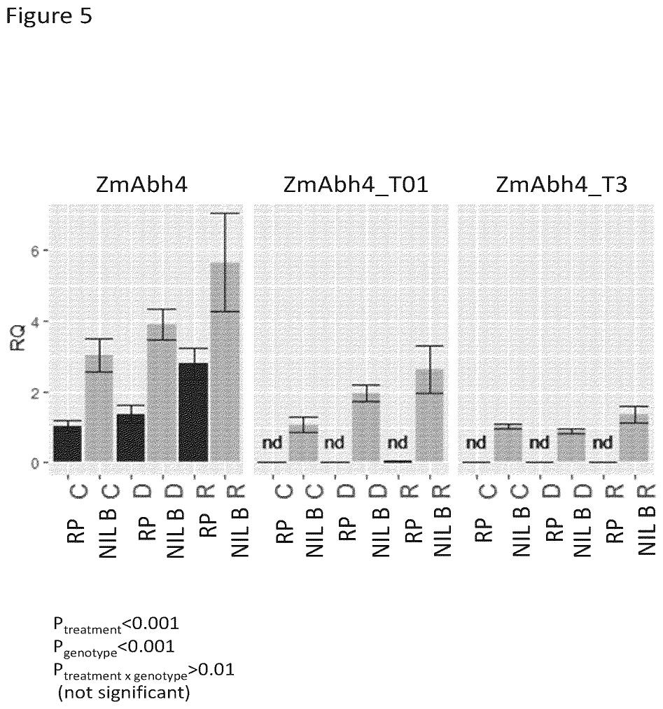

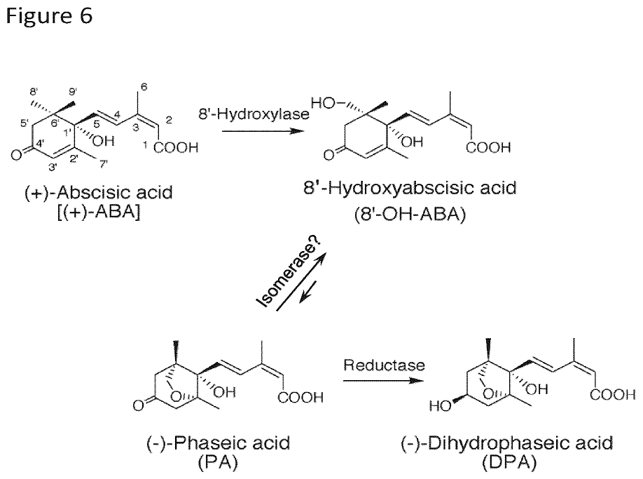

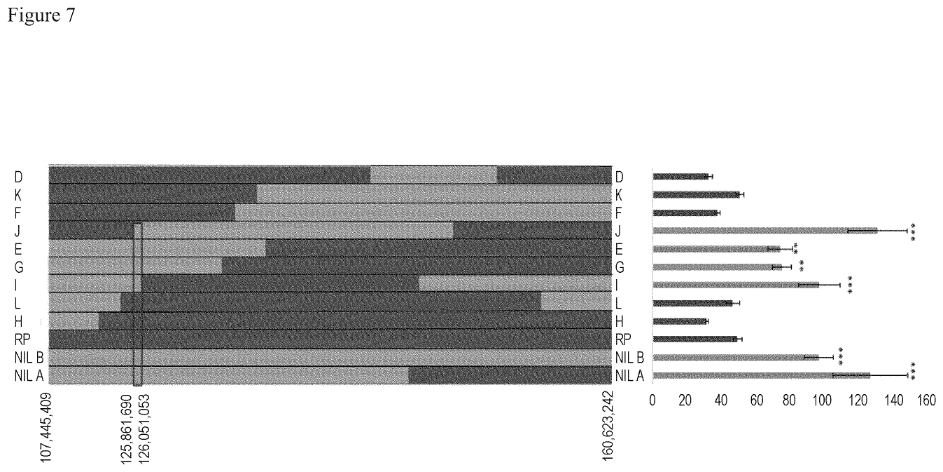

Based on the RNA-Seq data this gene (genomic DNA: SEQ ID NO: 9 (B73) and SEQ ID NO: 18 (PH207)) showed a significantly higher expression of the near isogenic line, carrying the DP allele, compared to RP in control, drought and re-watered conditions (FIG. 5). For this gene model three different transcript variants are described: T01 (transcript: SEQ ID NO: 10; cDNA: SEQ ID NO: 11) encoding the longest splice variant (expression of the DP allele higher than RP allele with FC of -1-2.5; protein: SEQ ID NO: 12) and T02 (transcript: SEQ ID NO: 13; cDNA: SEQ ID NO: 14) and T03 (transcript: SEQ ID NO: 16; cDNA: SEQ ID NO: 17) being shorter and encoding the same protein (expression of the DP T03 allele higher than the RP T03 allele with FC of ˜1-1.2; protein: SEQ ID NO: 15). Its localization on chromosome 7 from 125,973,529 to 125,976,469 on AGPv02 coordinates (from 129,916,913 to 129,919,853 on AGPv04 coordinates; from 126,143,580 to 126,147,082 on PH207) makes it a positional gene. Being attributed to a family of cytochrome P450 oxidases with putative function as abscisic acid 8′-hydroxylase 4, supports its role as a functional gene. Abscisic acid (ABA) is able to regulate stomatal aperture. As a gene being involved in the catabolism of ABA (FIG. 6), differences between one or all transcript isoforms lead to altered levels of ABA (FIG. 7) that affect stomatal aperture, conductance and in consequence might lead to differences in water use efficiency and carbon isotope discrimination. Correspondingly, the expression difference is particularly high for the long transcript isoform T01 between RP and DP. Analysis of ABA levels between RP and DP showed that RP has increased ABA levels compared to DP, which leads to faster closure of stomata and hence an early drought response. To validate ZmAbh4 as putative candidate gene, TILLING mutants were generated (Table 4) and allele variants of ZmAbh4 are tested.

TABLE 4a
Overview about the generated TILLING
mutants for the ZmAbh4 gene model

| ZmAbh4 | | N° mutants | | |
|---|---|---|---|---|
| Pop. PH207 | | Introns | Exons | AA Exchange |
| Winter 15/16 | Σ 12 | 7 | 5 | 1 |
| Summer 16 | Σ 15 | 4 | 11 | 3 |
| Winter 16/17 | Σ 19 | 4 | 15 | 6 |
| Summer 17 | Σ 44 | 17 | 27 | 11 |

TABLE 4b
Characterization of selected TILLING mutants of population
PH207. AA = amino acid, wt = wildtype, mut = mutant

| | codon | AA | codon | AA | | position | allele | allele |
|---|---|---|---|---|---|---|---|---|
| mutant code | wt | wt | mut | mut | location | in AA seq | wt | mut |
| PH207m015a | | | | | 2 bases | | | |
| | | | | | upstream | | | |
| | | | | | of exon 6 | | | |
| PH207m015b | ccc | pro | ctc | leu | exon 6 | 377 | C | T |
| PH207m015c | gga | gly | gaa | glu | exon 8 | 453 | G | A |
| PH207m015d | gtt | val | att | ile | exon 8 | 452 | G | A |
| PH207m015e | | | | | 4 bases | | C | T |
| | | | | | upstream | | | |
| | | | | | of exon 5 | | | |
| PH207m015f | gcc | ala | acc | thr | exon 4 | 252 | G | A |
| PH207m015g | cgt | arg | tgt | cys | exon 7 | 412 | C | T |
| PH207m015h | gac | asp | aac | asn | exon 4 | 307 | G | A |
| PH207m015i | gcg | ala | gtg | val | exon 4 | 289 | C | T |
| PH207m015j | ccg | pro | tcg | ser | exon 2 | 87 | C | T |
| PH207m015k | | | | | 1 base | | G | A |
| | | | | | down-stream | | | |
| | | | | | of exon 1 | | | |
| PH207m015l | gag | glu | aag | lys | exon 1 | 55 | G | A |
| PH207m015m | cct | pro | tct | ser | exon 1 | 45 | C | T |
| PH207m015n | gag | glu | aag | lys | | 370 | G | A |
| PH207m015o | gcc | ala | acc | thr | | 367 | G | A |
| PH207m015r | gtc | val | atc | ile | | 302 | G | A |
| PH207m015s | gac | asp | aac | asn | | 276 | G | A |
| PH207m015t | cgg | arg | cag | gln | | 161 | G | A |
| PH207m015u | cgc | arg | cac | his | | 150 | G | A |
| PH207m015v | ccc | pro | tcc | ser | | 146 | C | T |
| PH207m015w | ctt | leu | ttt | phe | | 83 | C | T |
| PH207m015x | ccc | pro | tcc | ser | | 64 | C | T |
| PH207m015y | ccc | pro | tcc | ser | | 40 | C | T |
| PH207m015z | | gly | | ser | exon | 422 | G | R |

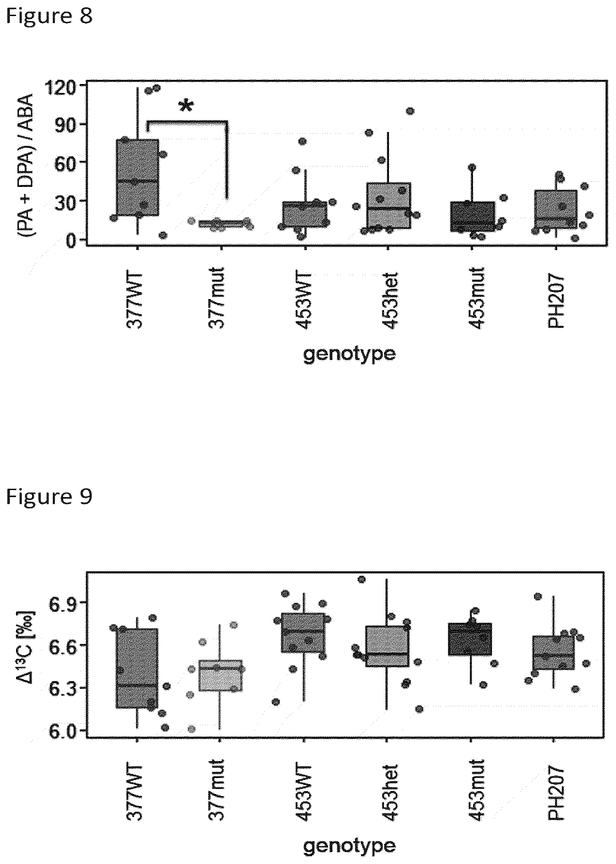

TILLING line PH207m015b (mutation P377L) was significantly different from its wild type regarding the ratio of products (phaseic acids and dihydrophaseic acid) to substrate (ABA) of the reaction catalyzed by ZmAbh4 (FIG. 8). However, there was no difference between PH207m15b and PH207.

For the line PH207m015c (mutation G453E), there was no difference in the ratio of products to substrate of the Abh4 reaction, to its wild type, to a line heterozygous for the mutation, and to PH207.

The carbon isotope discrimination (Δ¹³C) of leaves from the lines PH207m015Sb and PH207m015co did not differ from the discrimination in leaves from their wild types or PH207 (FIG. 9).

Possible reasons for the lack of phenotype observed in these two TILLING lines oan be either that the mutations under study are too mild to have an effect on the phenotype, that background mutations mask the phenotype, or hormone homeostasis in these lines is maintained by the regulation of other factors.

The rest of the TILLING lines will be further characterized.

In addition to the TILLING approaches, functional validation of ZmAbh4 is conducted via genetically modified organisms (GMOs).

In this respect, the dent genotype A188 was used as transformation background to achieve a strong constitutive overexpression of the ZmAbh4 gene by integrating a codon optimized ZmAbh4 gene under the control of the monocot ubiquitin promoter into the A188 genome and selecting for plants homozygous for an integration of this heterologous nucleotide. Table 5 gives an overview about number of seeds from transformants at the T1 generation that are still heterozygous for the integration.

Overexpression of ZmAbh4 is expected to reduce in planta ABA levels and thereby induce higher stomatal conductance due to extended opening of stomata under drought conditions.

Silencing of all ZmAbh family members including ZmAbh4 is conducted by expressing a heterologous hairpin construct in A188. T2 homozygous seed are generated and 11 events are at T1 stage. Silencing of ZmAbh4 is expected to increase ABA levels and result in an early drought response with low stomatal conductance and lower carbon isotope composition.

TABLE 5
Overview about the status of generated
GMO resources for ZmAbh4
ZmAbh4 OX UBI

| seed lot identifier | amount kernels | generation |
|---|---|---|
| MTR0349-T-002 | 19 | T1 |
| MTR0349-T-005 | 3 | T1 |
| MTR0386-T-004 | 5 | T1 |
| MTR0386-T-009 | 16 | T1 |
| MTR0389-T-001 | 46 | T1 |
| MTR0389-T-002 | 72 | T1 |
| MTR0386-T-031 | 17 | T1 |
| MTR0386-T-035 | 23 | T1 |
| MTR0386-T-040 | 15 | T1 |

ZmAbh4 Fam. RNAi
11 events @ T1

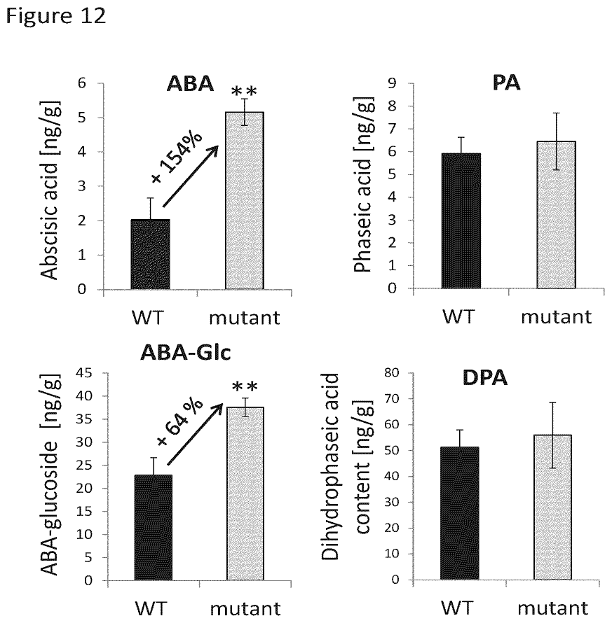

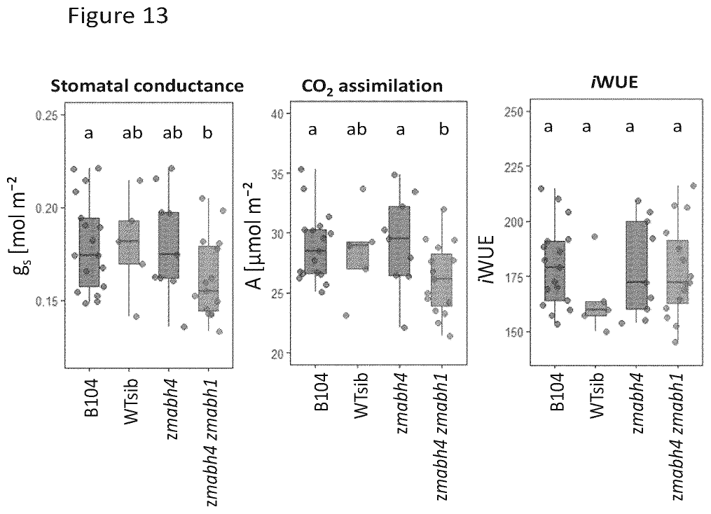

Constructs to knock-out the ZmAbh gene family using CRISPR/Cas9 were generated. Thereof one construct, encoding four guide RNAs, two targeting ZmAbh4, two targeting ZmAbh1 (deletions will alter 67% and 84% of the amino acid sequences, respectively), was used for transforming maize inbred line B104. Transformation was performed by VIB Center for Plant Systems Biology, Ghent, Belgium. Six independent events with mutations in ZmAbh4 were recovered. Thereof three events showed additional mutations in ZmAbh1. Plants originating from five events were genotyped and phenotyped. Preliminary results of the phenotyping of the T1 generation showed an 2.5× increase in ABA content in leaves of plants carrying two mutant alleles of ZmAbh4 (n=3) compared to plants carrying two wildype alleles (n=4, FIG. 12). The increase in ABA glucoside in the mutants and the unchanged levels of the products of ABA 8′-hydroxylation (PA, DPA, FIG. 12) indicate, that the plants use the glucoside to inactivate ABA instead of the hydroxylation, which might be impaired in the mutants. However, this is in contrast to the comparison of NIL B to RP, where differences in phaseic acid and dihydrophaseic acid levels were detected (FIG. 7). In addition the gas exchange measurements of mutants in this preliminary phenotyping did show differences to the wild type only in zmabh4 zmabh1 double mutants, not in the zmabh4 single mutants. However, many of the single mutants are heterozygous for the mutation, still carrying a wild type allele, while the proportion of homozygously mutated plants is higher in the double mutants. Still this observation could indicate that zmabh4 mutations can be compensated by ZmAbh1 in the background of B104.

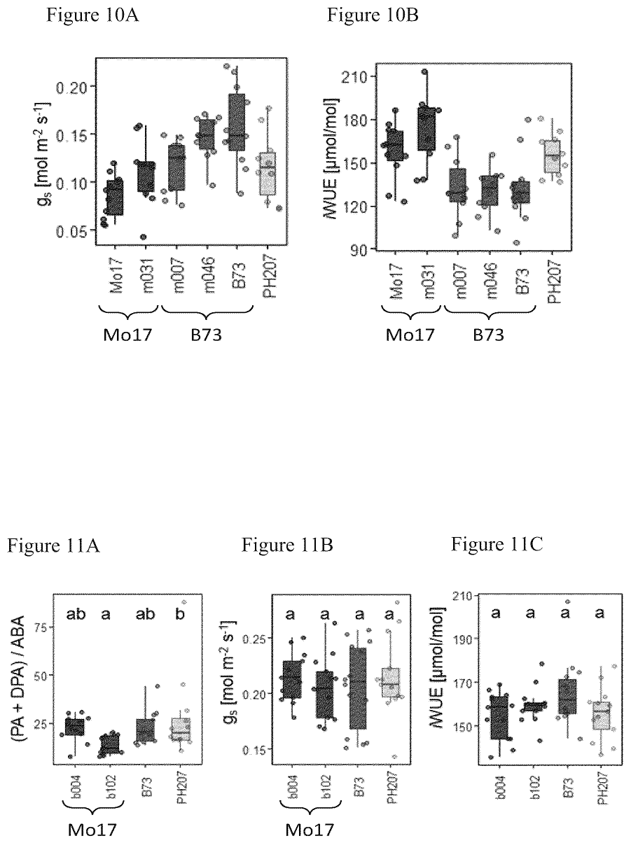

The ZmAbh4 alleles of near isogenic lines originating from crosses of the inbred lines B73 and Mo17 (Eichten et al. (2011) B73-Mo17 near-isogenic lines demonstrate dispersed structural variation in maize. In: Plant Physiol. 156 (4), S. 1679-1690. DOI: 10.1104/pp. 111.174748.) had an influence on stomatal conductance (gs) and instantaneous water use efficiency (NVUE) in the background of Mo17 (FIG. 10A, B) but not in the background of B73 (FIG. 11 B, C). This is an indication that Abh4 or at least the region around Abh4 is causative for the phenotypic differences in the background of Mo17. In the background of B73, maintained ABA catabolism rates (FIG. 11 A) in NILs explain the lack of phenotype in the gas exchange data.

### ZmWEB1

The gene (B73: genomic DNA: SEQ ID NO: 24; cDNA: SEQ ID NO: 25; protein: SEQ ID NO: 26; PH207: genomic DNA: SEQ ID NO: 27; cDNA: SEQ ID NO: 28; protein: SEQ ID NO: 29) shows higher expression in DP than RP in control conditions with FC of 4.92. Its localization on chromosome 7 from 126,142,402 to 126,145,382 on AGPv02 coordinates (from 130,051,739 to 130,054,355 on AGPv04; from 126,226,508 to 126,229,120 on PH207) makes it a positional gene. Its closest homologue in Arabidopsis thaliana (AT2G26570) is known as WEAK CHLOROPLAST MOVEMENT UNDER BLUE LIGHT-like protein (WEB1). This protein encodes a coiled-coil protein that, together with another coiled-coil protein WEB2/PMI2 (At1g66840), maintains the chloroplast photo-relocation movement velocity (Kodama et al., 2010 PNAS). Chloroplasts move toward weak light (accumulation response) and away from strong light (avoidance response). The fast and accurate movement of chloroplasts in response to ambient light conditions is essential for efficient photosynthesis and photodamage prevention in chloroplasts. Allelic differences in this gene influence the photosynthetic response and thereby also influence photosynthetic and stomatal parameters, which again leads to altered carbon isotope discrimination. Furthermore, its prominent expression in anthers might also play a role in the processes of flowering and the subsequent kernel formation and grain filling.

### GRMZM2G397260

No expression differences are observed between RP and DP for this gene (B73: genomic DNA: SEQ ID NO: 32; cDNA: SEQ ID NO: 33; protein: SEQ ID NO: 34). However, the gene is shown to be highly expressed in mature leaves in B73 (Sekhon et al., 2011). Its localization on chromosome 7 from 126,103,570 to 126,104,295 on AGPv02 coordinates (from 130047983 to 130048708 on AGPv04 coordinates) makes it a positional gene. No functional annotation is available for this gene. However, it seems to be a maize-specific gene as no significant homologies to other gene models could be detected.

### ZmHsftf21

No expression differences are observed between RP and DP for this gene (B73: genomic DNA: SEQ ID NO: 36; cDNA: SEQ ID NO: 37; protein: SEQ ID NO: 38; PH207: genomic DNA: SEQ ID NO: 39; cDNA: SEQ ID NO: 40; protein: SEQ ID NO: 41). Its localization on chromosome 7 from 125.861.349 to 125.865.050 on AGPv02 coordinates (from 129,797,898 to 129,801,599 on AGPv04 coordinates; from 126,047,960 to 126,052,077 on PH207 coordinates) makes it a positional gene. It encodes a Heat shock protein transcription factor 21, whose function is related to response to water deprivation and it is expressed in mature leaves in B73 (Sekhon et al., 2011), which makes it also a functional gene.

The recombinants are analysed for δ13C, leaf growth sensitivity to drought, whole plant water use efficiency (WUEplant), stomatal density, ABA leaf content.

Further Marker/Phenotype Correlations within the Set of Identified Recombinants

In order to genetically dissect the association of several drought-related traits to the genomic segment on chromosome 7, two consecutive greenhouse and one field experiment were performed. NIL B and the nine recombinant NILs (D-L), carrying small overlapping introgressions covering the target region were phenotyped together with their recurrent parent (RP). Ten plants per genotype were used in each of the two greenhouse experiments. Climate conditions were monitored (25-33° C./19-20° C. d/n, 400 μmol m⁻² s⁻¹ PAR, 40% RH) and supplemental light was used during the experiments. Two-week old single seedlings (developmental stage V3) were planted in 10 I pots, containing the same amount of sieved homogeneous soil and the same soil water content (SWC) organized in a randomized complete block design.

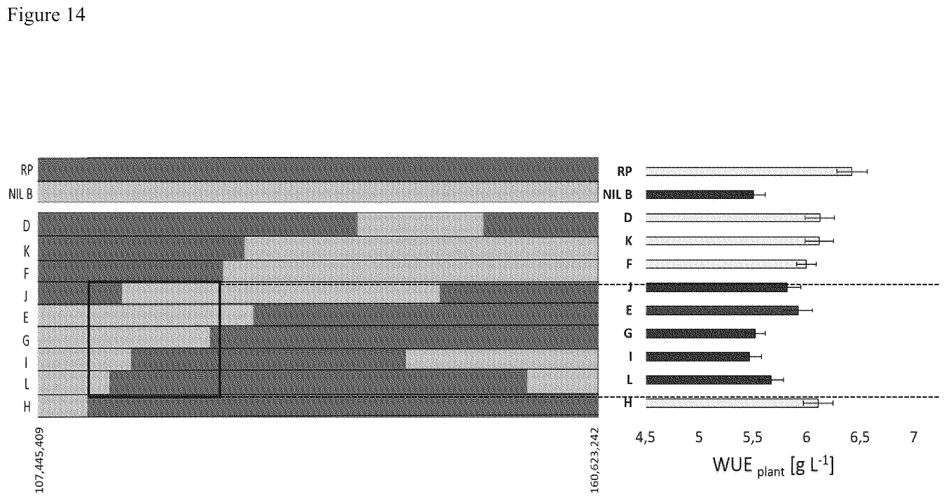

In the first experiment, whole-plant water use efficiency (WUEₚₗₐₙₜ) was evaluated. Maize plants were subjected to progressive drought stress by withholding water for 6 weeks. Starting SWC (vol/vol) was approximately 85%. Plastic bags were used to cover the surface of the pots to avoid soil water evaporation and no further watering was applied until the end of the experiment. The experiment was ended when all plants stopped growing (developmental stage V9-V10), started senescing, and had consumed all of the available water. SWC was determined gravimetrically, by weighing the pots and the amount of water consumed by each plant was calculated as the difference from the initial pot weight at the beginning of the experiment. At the end of the experiment, above-ground material was harvested for biomass determination after drying the material for 1 week at 60° C. to achieve constant weight. As the experiment is destructive, initial mean dry biomass of additional 2-week old plants was determined and subtracted from the final biomass for each genotype. WUEₚₗₐₙₜ was calculated as the ratio dry biomass/consumed water at the end of the experiment (see FIG. 14).

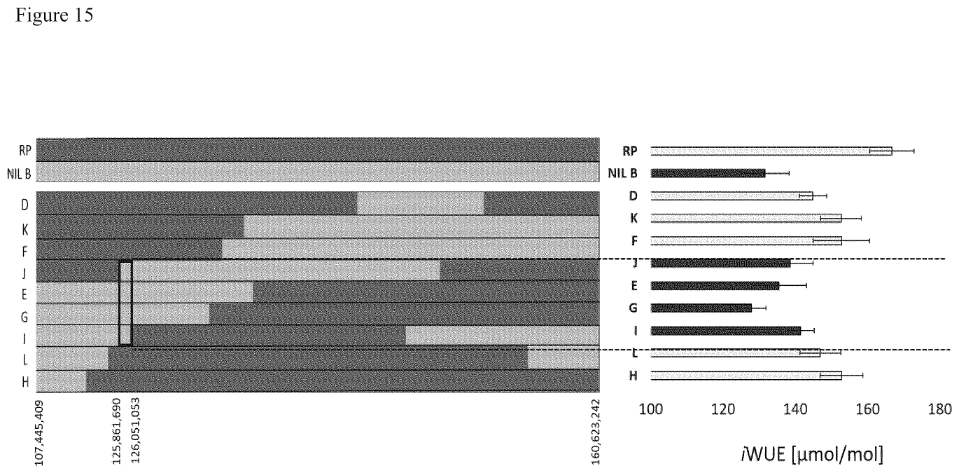

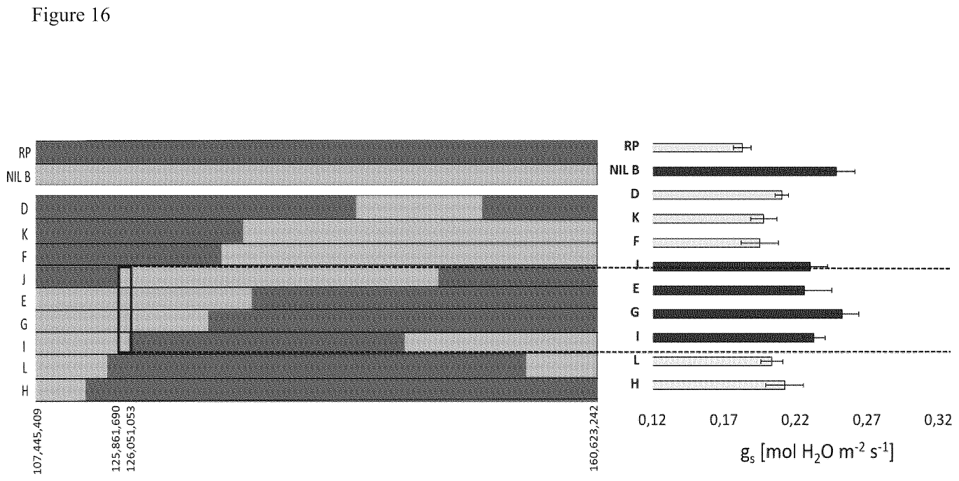

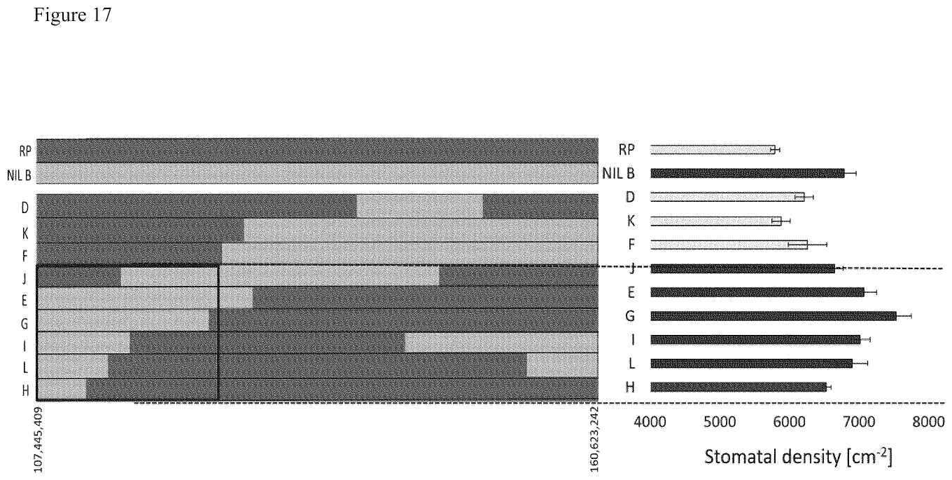

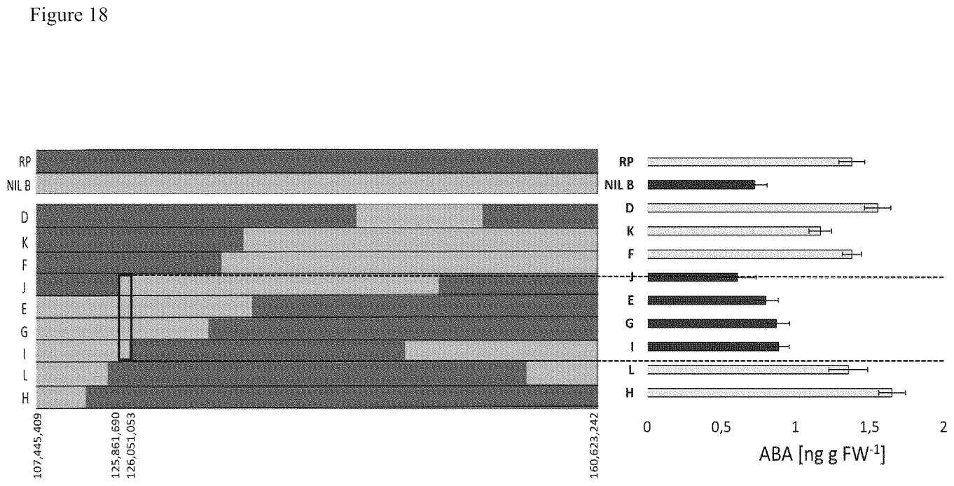

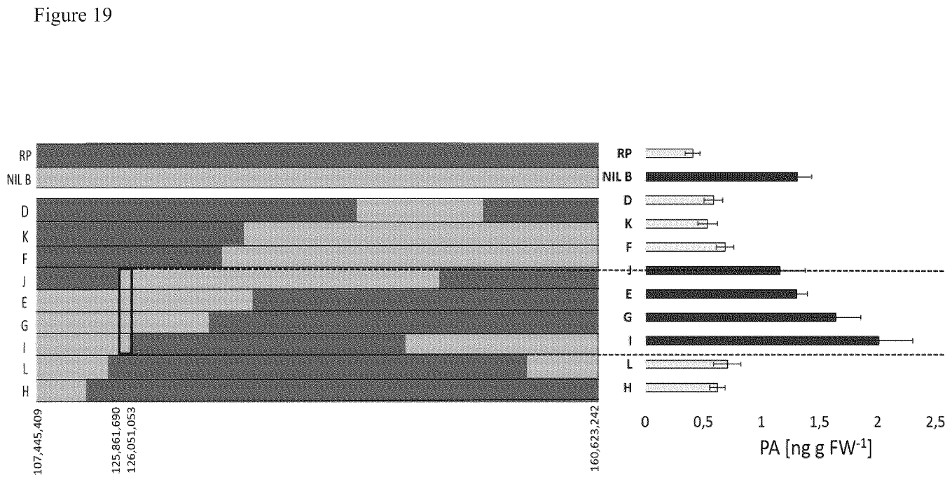

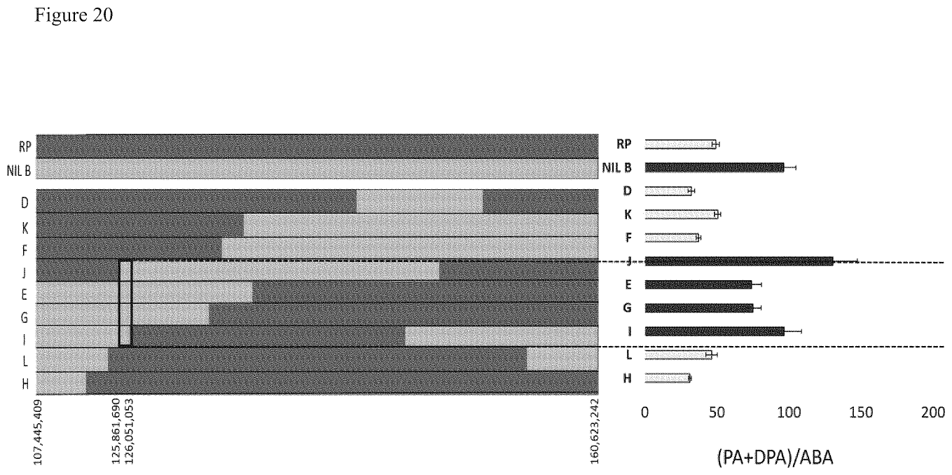

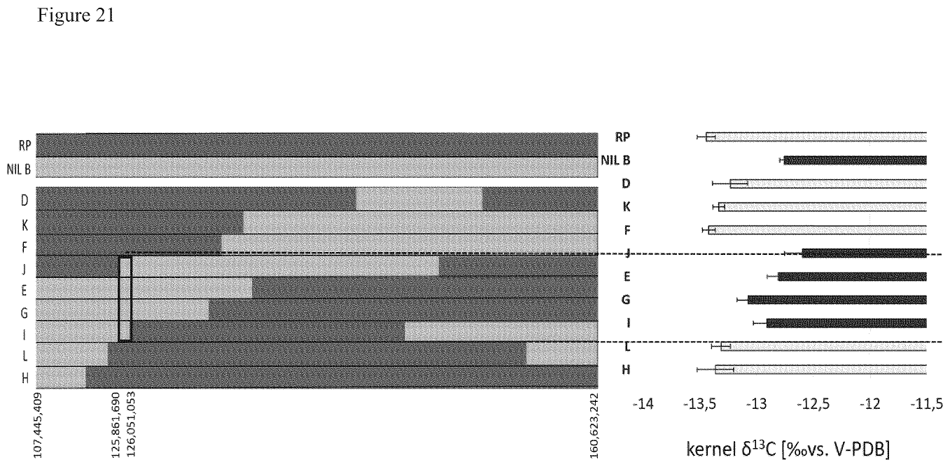

In the second greenhouse experiment, leaf gas exchange measurements were conducted using LI-6800 (LI-COR Biosciences GmbH, USA) on leaf 5 when it was fully developed (V5 developmental stage) to asses CO₂ assimilation (A) and stomatal conductance (gs) (FIG. 16) and calculate intrinsic WUE (iWUE) (FIG. 15) as the ratio between them. After that, leaf samples were taken from leaf 5 for stomatal density determination (FIG. 17). Nail varnish imprints were taken at three different places at the abaxial side in the middle of the leaf and were immobilized on the surface of a microscopic slide with a cellophane transparent tape. Pictures of the leaf epidermis were taken under a microscope. Stomata were counted and their number per leaf area was calculated. The whole leaf 5 was further instantly frozen in liquid nitrogen and further ground for preparing samples for hormone measurements by LC-MS/MS (abscisic acid (ABA) (FIG. 18) and its catabolites phaseic acid (PA) (FIG. 19) and dihydrophaseic acid (DPA) (FIG. 20)). Seeds were obtained from the same plants and 5-10 kernels per plant were ground together and used for carbon isotope composition (δ13C) measurements (FIG. 21).

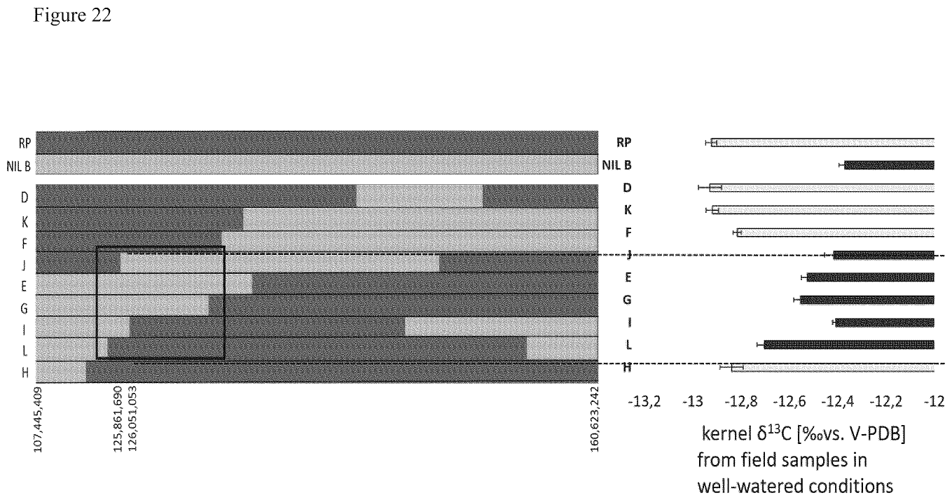

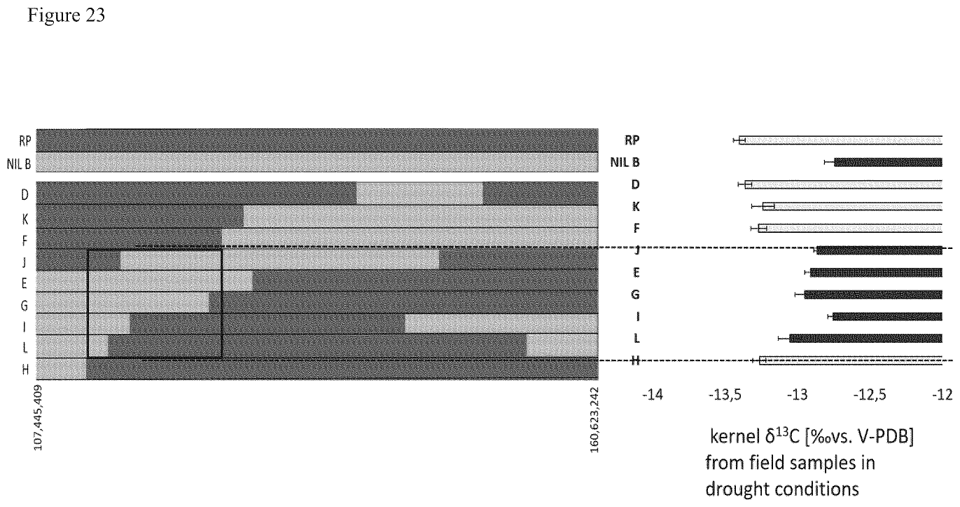

The field experiments were conducted in Freising, Germany. Plants were grown in a regularly well-watered field (48° 24′12.2″N, 11° 43′22.3″E) and in a rain-out shelter (48° 24′40.9″N, 11° 43′22.4″E) with reduced watering to achieve mild drought stress. The RP and the NILs were part of larger trials, which were laid out as randomized complete block designs with six replications per entry for both field and rain-out shelter. Each entry was planted in a single 1.2 m row with a 0.75 m distance between rows and intra-row spacing of 0.12 m, aiming at a plant density of 11 plants m-2. Application of herbicides and fertilizer followed good agricultural practice. All cobs per row were harvested manually and dried for 2 weeks at 30° C. before shelling. Grains were ground and used for analysis of δ13C (FIGS. 22 and 23).

1-6. (canceled)

7. A method of modifying a maize plant, comprising altering the expression level and/or activity of one or more genes located in a QTL located on chromosome 7, the QTL comprising a QTL allele, wherein the QTL allele is located on a chromosomal interval comprising molecular markers A and/or B, wherein molecular markers A and B are SNPs which are respectively C corresponding to position 125861690 and A corresponding to position 126109267 or which are respectively T corresponding to position 125861690 and G corresponding to position 126109267, referenced to the B73 reference genome AGPv2, optionally wherein the QTL allele is flanked by molecular markers A and/or B.

8. The method according to 7, wherein the one or more genes is selected from Abh4, CSLE1, WEB1, GRMZM2G397260, and Hsftf21.

9. The method according to claim 8, wherein;
Abh4 is selected from
(i) a nucleotide sequence comprising the nucleotide sequence of SEQ ID NO: 9;
(ii) a nucleotide sequence having the cDNA sequence of SEQ ID NO: 11, 14 or 17;
(iii) a nucleotide sequence encoding an amino acid sequence having the amino acid sequence of SEQ ID NO: 12 or 15;
(iv) a nucleotide sequence having at least 95% identity to the nucleotide sequence of SEQ ID NO: 9, 11, 14 or 17; and
(v) a nucleotide sequence encoding a polypeptide having at least 95% identity to the sequence of SEQ ID NO: 12 or 15;
CSLE1 is selected from
(i) a nucleotide sequence comprising the nucleotide sequence of SEQ ID NO: 1;
(ii) a nucleotide sequence having the cDNA sequence of SEQ ID NO: 2;
(iii) a nucleotide sequence encoding an amino acid sequence having the amino acid sequence of SEQ ID NO: 3;
(iv) a nucleotide sequence having at least 95% identity to the nucleotide sequence of SEQ ID NO: 1 or 2; and
(v) a nucleotide sequence encoding a polypeptide having at least 95% identity to the sequence of SEQ ID NO: 3;
WEB 1 is selected from
(i) a nucleotide sequence comprising the nucleotide sequence of SEQ ID NO: 24;
(ii) a nucleotide sequence having the cDNA sequence of SEQ ID NO: 25;
(iii) a nucleotide sequence encoding an amino acid sequence having the amino acid sequence of SEQ ID NO: 26;
(iv) a nucleotide sequence having at least 9% identity to the nucleotide sequence of SEQ ID NO: 24 or 25; and
(v) a nucleotide sequence encoding a polypeptide having at least 95% identity to the sequence of SEQ ID NO: 26;
GRMZM2G397260 is selected from
(i) a nucleotide sequence comprising the nucleotide sequence of SEQ ID NO: 32;
(ii) a nucleotide sequence having the cDNA sequence of SEQ ID NO: 33;
(iii) a nucleotide sequence encoding an amino acid sequence having the amino acid sequence of SEQ ID NO: 34;
(iv) a nucleotide sequence having at least 95% identity to the nucleotide sequence of SEQ ID NO: 32 or 33; and
(v) a nucleotide sequence encoding a polypeptide having at least 95% identity to the sequence of SEQ ID NO: 34;
Hsftf21 is selected from
(i) a nucleotide sequence comprising the nucleotide sequence of SEQ ID NO: 36;
(ii) a nucleotide sequence having the cDNA sequence of SEQ ID NO: 37;
(iii) a nucleotide sequence encoding an amino acid sequence having the amino acid sequence of SEQ ID NO: 38;
(iv) a nucleotide sequence having at least 95% identity to the nucleotide sequence of SEQ ID NO: 36 or 37; and
(v) a nucleotide sequence encoding a polypeptide having at least 95% identity to the sequence of SEQ ID NO: 38.

10. A method for generating a maize plant, comprising introducing into the genome of a plant a QTL located on chromosome 7, the QTL comprising a QTL allele, wherein the QTL allele is located on a chromosomal interval comprising molecular markers A and/or B, wherein molecular markers A and B are SNPs which are respectively C corresponding to position 125861690 and A corresponding to position 126109267 or which are respectively T corresponding to position 125861690 and G corresponding to position 126109267, referenced to the B73 reference genome AGPv2, optionally wherein the QTL allele is flanked by molecular markers A and/or B.

11. A method for obtaining a maize plant part, comprising:
(a) providing a first maize plant having a QTL or one or more molecular markers,
(b) crossing the first maize plant with a second maize plant,
(c) selecting progeny plants having the QTL or the one or more molecular markers, and
(d) harvesting the plant part from the progeny,
wherein the QTL is located on chromosome 7 and comprises a QTL allele, wherein the QTL allele is located on a chromosomal interval comprising molecular markers A and/or B, wherein molecular markers A and B are SNPs which are respectively C corresponding to position 125861690 and A corresponding to position 126109267 or which are respectively T corresponding to position 125861690 and G corresponding to position 126109267, referenced to the B73 reference genome AGPv2, optionally wherein the QTL allele is flanked by molecular markers A and/or B.

12-15. (canceled)

16. The method according to claim 7, wherein the QTL allele comprises molecular markers C, D, E, and/or F, wherein molecular markers C, D, E, and F are SNPs which are respectively A corresponding to position 125976029, A corresponding to position 127586792, C corresponding to position 129887276, and C corresponding to position 130881551, or which are respectively G corresponding to position 125976029, G corresponding to position 127586792, T corresponding to position 129887276, and T corresponding to position 130881551, referenced to the B73 reference genome AGPv2, optionally wherein the QTL allele is flanked by molecular markers A and/or F.

17. The method according to claim 7, wherein the expression level and/or activity of the one or more genes is decreased relative to a wildtype maize plant.

18. The method according to claim 7, wherein a gene of the one or more genes is mutated.

19. The method according to claim 18, wherein the mutation is an insertion of one or more nucleotides resulting in an altered expression of the gene or an altered activity of a polypeptide encoded by the gene.

20. A maize plant produced by the method of claim 7.
